(19) United States
(12) Patent Application Publication
(10) Pub. No.: US 2024/0238509 A1
(43) Pub. Date: Jul. 18, 2024

(54) Infusion Device Assembly

(71) Applicant: DEKA Products Limited Partnership, Manchester, NH (US)

(72) Inventors: Laura KESSEL, Somerville, MA (US); Benjamin W. JONES, Jr., Salisbury, NH (US); Eoin MCGROTTY, Manchester, NH (US); Kevin L. GRANT, Litchfield, NH (US); Cory L. ADAMS, Pembroke, NH (US)

(21) Appl. No.: 18/550,497

(22) PCT Filed: Mar. 15, 2022
(86) PCT No.: PCT/US22/71158

§ 371 Date: Sep. 14, 2023

Related U.S. Application Data
(60) Provisional application No. 63/161,570, filed on Mar. 16, 2021.

(51) Int. Cl. A61M 5/142

(52) U.S. Cl. CPC A61M 5/142; A61M 2005/14268; A61M 2205/123; A61M 2205/128; A61M 2207/10

(57) ABSTRACT

A cassette for a drug delivery device may comprise a cassette base portion including a reservoir recess surrounded by an attachment surface. The attachment surface may include a reservoir outlet flow path recessed therein. The cassette may further comprise a reservoir film coupled to the attachment surface which, together with the reservoir recess, defines a flexible reservoir. A portion of the reservoir film may have a preformed shape which mimics the contour of the reservoir recess. The preform shape may cause the reservoir film to be disposed adjacent the surface of reservoir recess when the reservoir is in an empty state. The cassette may further comprise at least one duct recessed into the surface of the reservoir recess. Each of the at least one duct may define a flow path which remains in fluid communication with the reservoir outlet flow path when the reservoir is in an empty state.

### CROSS-REFERENCE TO RELATED APPLICATIONS

This application claims the benefit of U.S. Provisional Patent Application Ser. No. 63/161,570 filed Mar. 16, 2021, entitled Infusion Device Assembly (Attorney Docket No. AA069), which is incorporated herein by reference in its entirety.

### BACKGROUND

Field of Disclosure: This disclosure relates to fluid infusion. More specifically, this disclosure relates to fluid infusion device assemblies.

### DESCRIPTION OF RELATED ART

Many potentially valuable medicines or compounds, including biologicals, are not orally active due to poor absorption, hepatic metabolism or other pharmacokinetic factors. Additionally, some therapeutic compounds, although they can be orally absorbed, are sometimes required to be administered so often it is difficult for a patient to maintain the desired schedule. In these cases, parenteral delivery is often employed or could be employed. Other medicines can be administered by routes other than parenteral, but the bioavailability of the drug varies from an ideal amount over time.

Effective parenteral routes of drug delivery, as well as other fluids and compounds, such as subcutaneous injection, intramuscular injection, and intravenous (IV) administration include puncture of the skin with a needle or stylet. Insulin is an example of a therapeutic fluid that is self-injected by millions of diabetic patients. Users of parenterally delivered drugs would benefit from a wearable device that would automatically deliver needed drugs/compounds over a period of time.

To this end, there have been efforts to design portable devices for the controlled release of therapeutics. Such devices are known to have a reservoir such as a cartridge, syringe, or bag, and to be electronically controlled. These devices suffer from a number of drawbacks including the malfunction rate. Reducing the size, weight and cost of these devices is also an ongoing challenge.

### SUMMARY

In accordance with an embodiment of the present disclosure a cassette assembly for a drug delivery device may comprise a cassette base portion including a reservoir recess surrounded by an attachment surface. The attachment surface may include a reservoir outlet flow path recessed therein. The cassette assembly may further comprise a reservoir film coupled to the attachment surface that, together with the reservoir recess, may define a flexible reservoir. A portion of the reservoir film may have a preformed shape which mimics the contour of the reservoir recess. The preform shape may cause the reservoir film to be disposed adjacent the surface of reservoir recess when the reservoir is in an empty state. The cassette assembly may further comprise at least one duct recessed into the surface of the reservoir recess. Each of the at least one duct may define a flow path which remains in fluid communication with the reservoir outlet flow path when the reservoir is in an empty state.

In some embodiments, the reservoir film may comprise an outcrop region included in the reservoir film. The outcrop region may form a seal over the reservoir outlet flow path when the reservoir film is coupled to the attachment surface. In some embodiments, the reservoir film may include at least two layers. In some embodiments, the reservoir film may be heat bonded to the attachment surface. In some embodiments, the cassette base portion may include a side wall having a fill port leading to an interior volume of the reservoir. In some embodiments, a basin may be recessed into the reservoir recess directly downstream of an inlet leading to the interior volume of the reservoir from the fill port. In some embodiments, at least one of the at least one duct may extend into communication with the basin. In some embodiments, the at least one duct may include a plurality of ducts which converge together at a confluence. The confluence may be disposed intermediate the reservoir outlet flow path and the ducts. In some embodiments, the at least one duct may include a first duct and a second duct which furcates off of the first duct. In some embodiments, the reservoir recess may include a wall which extends from a bottom surface of the reservoir recess to the attachment surface and the at least one duct includes a duct which is disposed along a portion of a perimeter of the bottom surface adjacent the wall. In some embodiments, the duct disposed along a portion of the perimeter may be disposed along a majority of the perimeter. In some embodiments, the at least one duct may have a variable width. In some embodiments, the reservoir recess may include a wall which extends from a bottom surface of the reservoir recess to the attachment surface and the at least one duct may include a portion recessed into the bottom surface and a second portion recessed into the wall. In some embodiments, the first portion of the at least one duct may have a first width and the second portion of the at least one duct has a variable width. In some embodiments, the width of the second portion of the at least one duct may taper from a first width to a second width smaller than the first width. In some embodiments, the first portion of the at least one duct may be the second width. In some embodiments, the at least one duct may include a plurality of ducts which extend from a confluence region at regular angular intervals. The confluence region may be disposed intermediate the plurality of ducts and the reservoir outlet flow path. In some embodiments, the reservoir outlet flow path may include an air trap. In some embodiments, the air trap may include at least one of a screen or mesh.

In accordance with another embodiment of the present disclosure a cassette assembly for a drug delivery device may comprise a cassette base portion including a reservoir recess surrounded by an attachment surface. The cassette base portion may further include a weld surface along a portion of the periphery of the cassette base portion and a plurality of locating pins. The cassette assembly may further comprise a reservoir film coupled to the attachment surface which, together with the reservoir recess, may define a flexible reservoir. The cassette assembly may further comprise a cassette top portion including a number of locating pin receptacles and a peripheral energy director. The energy director may be aligned with the weld surface and a portion of the reservoir film bonded to the attachment surface when the locating pins are disposed within the locating pin receptacles. The energy director may be welded to the weld surface and the portion of the reservoir film.

In some embodiments, the energy director may comprise a triangular cross section. In some embodiments, the energy director may be sonically welded to the weld surface and the portion of the reservoir film. In some embodiments, the energy director may be ultrasonically welded to the weld surface and the portion of the reservoir film. In some embodiments, the reservoir film may include at least two layers. In some embodiments, the cassette top portion may include a bay including a number of retention tabs. In some embodiments, the cassette assembly may further comprise a reservoir cover having a number of latch clips. The latch clips may be configured to cooperatively engage the retention tabs of the bay. The reservoir cover may be coupled to the cassette top portion when the latch clips cooperatively engage the retention tabs.

In accordance with another embodiment of the present disclosure a cassette assembly for a coupling to a reusable housing assembly of a drug delivery device may comprise a cassette base portion. The cassette assembly may further comprise a reservoir defined by a recess in the cassette base portion and a segment of reservoir film coupled to an attachment surface disposed around the recess. The cassette assembly may further comprise a cassette top portion covering the cassette base portion and including an inspection bay which is disposed over the entirety of the reservoir. The inspection bay may include a number of coupling elements. The cassette assembly may further comprise a reservoir cover including a number of cooperative coupling elements configured to engage with the coupling elements of the inspection bay to couple the reservoir cover in place within the inspection bay. The reservoir may be directly accessible through the inspection bay prior to installation of the reservoir cover.

In some embodiments, the cassette base portion may be constructed of a transparent material. In some embodiments, the cassette base portion may be constructed of a clear material. The material may be sufficiently translucent to allow for inspection by machine or other mechanisms during the filling process. In some embodiments, the cassette base portion may be constructed of a light colored material. In some embodiments, the reservoir cover may be a solid piece of material. In some embodiments, the coupling elements may be retention tabs and the cooperative coupling elements may be latch clips. In some embodiments, one of the latch clips may be disposed at an unsupported end of a cantilevered member of the reservoir cover. In some embodiments, the coupling elements and cooperative coupling elements may be configured to engage via a snap fit. In some embodiments, the reservoir may be a prefilled reservoir containing a drug. In some embodiments, the cassette may include a flow path extending from the reservoir to delivery tubing attached to the cassette. The cassette may further include an occluder assembly disposed at a section of the flow path. The occluder assembly may be actuatable between an occluding state and a flow permitting state.

In accordance with yet another embodiment of the present disclosure a prefilled cassette assembly for a drug delivery device may comprise a cassette base portion. The cassette may further comprise a reservoir defined by a recess in the cassette base portion and a piece of reservoir film attached to based portion at an attachment surface surrounding the recess. The reservoir may be filled with a medicament. The cassette may further comprise a flow path extending from the reservoir to an outlet of the cassette. The flow path may be defined at least in part by a membrane cover along a segment of the flow path. The cassette assembly may further include an occluder assembly actuatable between a flow permitting state and an occluding state in which the reservoir and a first portion of the flow path are isolated from a second portion of the flow path downstream of the first. The segment of the flow path defined at least in part by the membrane cover may be part of the second portion of the flow path. The first portion of the flow path may be constructed of material having long term compatibility with the medicament and at least the membrane cover is constructed of material having short term compatibility with the medicament.

In some embodiments, the reservoir film may be a layered film. In some embodiments, the second portion of the flow path may include at least one valve station. In some embodiments, the membrane cover may be coupled in place over the valve station so as to form a fluid tight seal. In some embodiments, the second portion of the flow path may include at least one pumping chamber. In some embodiments, the membrane cover may be coupled in placed over the valve station so as to form a fluid tight seal. In some embodiments, the occluder assembly may include an occluder diaphragm and an occluder actuator. In some embodiments, the cassette assembly may further comprise a threaded port and in the occluding state the occluder actuator may be threaded into the flow path and the occluder diaphragm is actuated to occlude the flow path. In some embodiments, the cassette assembly may further comprise a threaded port configured to receive the occlude actuator and the occluder assembly may be configured to transition from the occluding state to the flow permitting state by extracting the occlude actuator from the threaded port. In some embodiments, the occlude actuator may be configured to prevent mating of the cassette assembly with a reusable housing assembly when present in the cassette assembly. In some embodiments, at least a portion of the occluder diaphragm may be constructed of material having long term compatibility with the medicament. In some embodiments, the occluder assembly may be constructed of material having long term compatibility with the medicament. In some embodiments, at least a portion of the occluder assembly may be constructed of material having long term compatibility with the medicament. In some embodiments, the medicament may be a drug for an endocrine disorder. In some embodiments the medicant may be treprostinil. In some embodiments, the medicament may be selected from a group consisting of insulin and an insulin analog. In some embodiments, the medicament may be proteinaceous. In some embodiments, the medicament may include a polypeptide having a tertiary structure with at least one hydrophobic region.

In accordance with an embodiment of the present disclosure a prefilled cassette assembly for a drug delivery device may comprise a housing. The cassette assembly may further comprise a reservoir containing a medicament. The cassette assembly may further comprise a flow path extending from the reservoir to an outlet of the cassette assembly. The flow path may include a number of valve stations and a pumping chamber. The cassette assembly may further comprise an occluder assembly including an occluder diaphragm and an occluder actuator in threaded engagement with a threaded port in the cassette assembly. The occluder assembly may have an occluding state in which the occluder actuator may be in a first position in the threaded port and presses the occlude diaphragm into sealing engagement with an occluder seat to occlude the flow path. The occluder assembly may have a flow permitting state in which the occlude actuator may be unthreaded out of the threaded port from the first position to at least a second position to relieve pressure against the occluder diaphragm.

In some embodiments, the reservoir may be defined by a recess in the cassette base portion and a piece of reservoir film attached to based portion at an attachment surface surrounding the recess. In some embodiments, the occluder assembly may gate flow from a reservoir outlet flow path to a remainder of the flow path including the pumping chamber and the valve stations. In some embodiments, the occluder diaphragm may be captured between a cassette base portion and a cassette top portion of the housing. In some embodiments, the occluder assembly may be included in a depression or occluder seat in a face of the cassette base portion and the occluder diaphragm may create a fluidic seal about a periphery of the depression so as to form a sealed occluder volume. In some embodiments, the depression may include an occluder outlet. In some embodiments, the occluder outlet may be partially surrounded by a wall. The wall may extend from the depression a distance greater than the height of the occluder assembly. In some embodiments, the occluder assembly may be a volcano type arrangement. In some embodiments, the pumping chamber and valve stations, with the exception of the occluder assembly, may be covered by a valve membrane cover. In some embodiments, the housing may include a cassette base portion and a cassette top portion and the valve membrane cover may include an outcrop region which forms a seal over at least one flow channel recessed into a face of the cassette base portion when the cassette base portion and cassette top portion are coupled to one another. In some embodiments, the occluder actuator may include a knob and a rod projection from the rod. In some embodiments, the rod may include a number of ramp features disposed at a terminal end of the rod opposite the knob. The ramp features may have a pitch selected to cooperate with threading in the threaded port. In some embodiments, the ramp features may be disposed at even angular increments. In some embodiments, the threading in the threaded port includes a shelf section. The ramp features of the occlude actuator may be parked on the shelf feature when the occluder actuator is in the first position.

In accordance with another embodiment of the present disclosure, a cassette assembly for a drug delivery device may comprise a cassette base portion including a side wall and a peripheral recess adjacent the side wall. The cassette assembly may further comprise a cassette top portion coupled to the cassette base portion. The cassette assembly may further comprise a coupling comprised of a number of tabs projecting from the cassette top portion overhanging the peripheral recess and a number of rotation stops included on the cassette base portion and projecting into the recess. The rotation stops and tabs may cooperate to form a number of mating tracks. The cassette assembly may further comprise a collapsible reservoir. The cassette assembly may further comprise a fill port extending from the side wall to the reservoir disposed in between two of the mating tracks. Upon introduction and rotation of a mating finger on a reusable housing assembly of the delivery device in each mating track, the cassette assembly may be configured to couple with the reusable housing assembly.

In some embodiments, the tabs may include a detent region. In some embodiments, the tabs may include a ramped section. In some embodiments, the cassette top portion may include a sealing ring. In some embodiments, the cassette top portion may include a sealing ring and the tabs may include a ramped section. The sealing ring may be configured to be compressed by the reusable housing assembly when the coupling fingers of the reusable housing assembly are rotated along the ramped section of the tabs to releasably connect the cassette assembly to the reusable housing assembly. In some embodiments, the fill port may include a septum. In some embodiments, the fill port may include a guard wall adjacent the reservoir. In some embodiments, the guard wall may include an inlet which places a bore of the fill port into fluid communication with an interior volume of the reservoir. In some embodiments, the rotation stops may be disposed perpendicularly to the tabs. In some embodiments, the collapsible reservoir may comprise a reservoir recess in the cassette base portion and a piece of reservoir film sealing attached to the cassette base portion at an attachment surface surround the reservoir recess. In some embodiments, the reservoir recess may include a recessed basin directly downstream of the fill port.

In accordance with an embodiment of the present disclosure a cassette assembly for a drug delivery device may comprise a cassette base portion. The cassette assembly may further comprise a reservoir formed by a reservoir recess in the cassette base portion and a piece of reservoir film attached to the cassette base portion at an attachment surface surrounding the reservoir recess. The cassette assembly may further comprise a valve membrane cover. The cassette assembly may further comprise a channel film seal. The cassette assembly may further comprise a flow path extending from the reservoir to an outlet of the cassette assembly. The flow path may include a number of valve stations, at least one pump chamber, at least one channel recessed into each of a first face and opposing second face of the cassette base portion, and a number of conduits extending through the cassette base portion connecting the channels on the first and second face of the cassette base portion. The valve membrane cover may form a seal around the number of valve stations and the at least one pump chamber. The channel film seal may seal the at least one channel recessed into the second face of the cassette base portion. The at least one channel recessed into the first face of the cassette base portion may be sealed by at least one of the valve membrane cover and the reservoir film.

In some embodiments, the reservoir film and channel film seal may be constructed of the same material. In some embodiments, the reservoir film may be a multilayer film. In some embodiments, the reservoir film may be heat bonded to the second face of the cassette assembly. In some embodiments, the reservoir film may be heat bonded to the attachment surface. In some embodiments, the at least one channel in the first face of the cassette base portion may include an air trap. In some embodiments, the at least one channel recessed into the first face of the cassette base portion may include a reservoir outlet channel recessed into the attachment surface. The reservoir film may form a seal over the reservoir outlet channel.

In some embodiments, the at least one channel recessed into the first face of the cassette base portion may include a channel which may be sealed over by an outcrop region included in the valve membrane cover. In some embodiments, the flow path may further include an occluder assembly. In some embodiments, the reservoir may be prefilled by a manufacturer. In some embodiments, the at least one channel recessed into the second face of the cassette base portion as well as the channel film seal may be surrounded by a raised rim wall. In some embodiments, the cassette assembly may further comprise a cassette top portion coupled to the cassette base portion. The valve membrane cover may be compressed between the cassette base portion and cassette top portion to form a fluid tight seal. In some embodiments, the cassette assembly may further comprise a cassette top portion coupled to the cassette base portion by a sonic weld. At least a portion of the sonic weld may be formed between the cassette top portion and the reservoir film.

In accordance with another embodiment of the present disclosure, a method of filling a reservoir for a cassette assembly of a drug delivery device may comprise actuating an occluder assembly of the cassette assembly to occlude a flow path leading from the reservoir to an outlet of the set. The method may further comprise loading fluid into the reservoir through a fill port. The method may further comprise imaging the reservoir. The method may further comprise determining whether the reservoir meets acceptability criteria by analyzing at least one image of the reservoir. The method may further comprise installing a reservoir cover in an inspection bay which provides visual access to the reservoir.

In some embodiments, imaging the reservoir may comprise photographing the reservoir through the inspection bay. In some embodiments, determining whether the reservoir meets acceptability criteria may comprise analyzing the at least one image to determine if particulates are present. In some embodiments, determining whether the reservoir meets acceptability criteria may comprise analyzing the at least one image to determine if an amount of air greater than a predefined threshold is present. In some embodiments, actuating the occluder assembly may comprise installing a removable occluder actuator in a port of the cassette assembly. In some embodiments, actuating the occluder assembly may comprise displacing an occluder diaphragm against a reservoir outlet valve to close off flow through the reservoir outlet valve. In some embodiments, installing the reservoir cover in the inspection bay may comprise coupling the reservoir cover to the cassette assembly via snap fit.

In accordance with another embodiment of the present disclosure, a cassette assembly for a drug delivery device may comprise a cassette base portion. The cassette assembly may further comprise a cassette top portion coupled to the cassette base portion. The cassette assembly may further comprise a collapsible reservoir. The cassette assembly may further comprise an outlet. The cassette assembly may further comprise a flow path extending from the collapsible reservoir to the outlet. The cassette assembly may further comprise a fill port extending from a side wall of the cassette base portion to the reservoir, the fill port including at least one of an access restriction configuration and a reuse prevention configuration.

In accordance with another embodiment of the present disclosure a cassette assembly for a drug delivery device may comprise a cassette base portion. The cassette assembly may further comprise a reservoir defined by a reservoir recess in the cassette base portion and a reservoir film attached to the cassette base portion at an attachment surface surrounding the reservoir recess. The reservoir may be filled with a medicament. The cassette assembly may further comprise a flow path extending from the reservoir to an outlet of the cassette assembly. The flow path may be defined at least in part by a membrane cover along a segment of the flow path. The cassette assembly may further comprise an occluder assembly configured for actuation between a flow permitting state and an occluding state in which the reservoir and a first portion of the flow path are isolated from a second portion of the flow path downstream of the first. The segment of the flow path may define at least in part by the membrane cover being part of the second portion of the flow path.

In some embodiments, the first portion of the flow path may be constructed of material having long term compatibility with the medicament and at least the membrane cover may be constructed of material having short term compatibility with the medicament. In some embodiments, the reservoir film may be a layered film. In some embodiments, the second portion of the flow path may include at least one valve station. In some embodiments, the membrane cover may be coupled in place over the valve station so as to form a fluid tight seal. In some embodiments, the second portion of the flow path may include at least one pumping chamber. In some embodiments, the membrane cover may be coupled in placed over the valve station so as to form a fluid tight seal. In some embodiments, the occluder assembly may include an occluder diaphragm and an occluder actuator. In some embodiments, the cassette assembly may further comprise a threaded port and in the occluding state the occluder actuator may be threaded into the flow path and the occluder diaphragm may be actuated to occlude the flow path. In some embodiments, the cassette assembly may further comprise a threaded port configured to receive the occluder actuator and the occluder assembly may be configured to transition from the occluding state to the flow permitting state by extracting the occluder actuator from the threaded port. In some embodiments, the occluder actuator may be configured to prevent mating of the cassette assembly with a reusable housing assembly when present in the cassette assembly. In some embodiments, at least a portion of the occluder diaphragm may be constructed of material having long term compatibility with the medicament. In some embodiments, the occluder assembly may include an occlude actuator which is removable from the cassette assembly. The occluder assembly may be in a flow permitting state when the occluder actuator is removed from the cassette assembly. In some embodiments, the occluder assembly may include a rotatable occluder actuator. The occluder assembly configured to transition from the occluding state to the flow permitting state upon rotation of the occluder actuator. In some embodiments, the occluder assembly may include a displaceable occluder actuator. The occluder assembly may be configured to transition from the occluding state to the flow permitting state upon translational displacement of the occluder actuator. In some embodiments, the occluder assembly may include an occluder shuttle disposed in an occluder channel. In some embodiments, the occlude shuttle may comprise at least one sealing interface which generates a fluidic seal against the wall of the occluder channel. In some embodiments, in the occluding state the at least one sealing interface may be disposed intermediate an opening of the first portion of the flow path to the occluder channel and an opening of the second portion of the flow path to the occluder channel. In some embodiments, the occluder shuttle may be covered by an occluder shuttle diaphragm. In some embodiments, the occluder shuttle may be a compressible elastomer. In some embodiments, the occluder shuttle may be compressible and may include an enlarged segment. The occluder channel may include a step between a wide section and narrow section. The enlarged segment of the occluder shuttle may be wider than the narrow section in an uncompressed state. In some embodiments, the occluder assembly may be constructed of material having long term compatibility with the medicament. In some embodiments, at least a portion of the occluder assembly may be constructed of material having long term compatibility with the medicament. In some embodiments, the medicament may be a drug for an endocrine disorder. In some embodiments, the medicament may be selected from a group consisting of insulin and an insulin analog. In some embodiments the medicant may be treprostinil. In some embodiments, the medicament may be proteinaceous. In some embodiments, the medicament may include a polypeptide having a tertiary structure with at least one hydrophobic region. In some embodiments, the occluder assembly may include an occluder bung and a tapered port. The occluder bung may be retained in the tapered port via an interference fit. In some embodiments, the occluder assembly may include an occluder diaphragm. The occluder bung may compress and seal the occluder diaphragm against a reservoir outlet opening when retained in the tapered port. In some embodiments, the occluder bung may be constructed of compressible elastomer.

In accordance with another embodiment of the present disclosure, a cassette assembly for a drug delivery device may comprise a cassette base portion. The cassette assembly may further comprise a cassette top portion coupled to the cassette base portion. The cassette assembly may further compromise a cassette shell. The cassette shell may be made of a material compatible with contact with a user's skin and resistant to body oils, sweat, and lotions that may be applied to a user's skin. The cassette shell may cover substantially the bottom and sides of the coupled cassette top portion and cassette bottom portion, leaving the top open for attachment to the reusable housing assembly. The cassette shell may attach to the coupled cassette base portion and cassette top portion with a snap fit arrangement of tabs and slots. The cassette assembly may further comprise a collapsible reservoir defined by a recess in the cassette base portion and covered with a reservoir film. The cassette assembly may further comprise an outlet. The cassette assembly may further comprise a flow path extending from the collapsible reservoir to the outlet. The reservoir may have a fill port extending from the side of the cassette base portion. The reservoir may be filled with medicant and the fill port may be closed with a reservoir plug. The cassette shell can cover the fill port and further comprise a shell projection that helps hold the reservoir plug in position during transport and use of the cassette assembly.

In accordance with another embodiment of the present disclosure, a drug delivery system may comprise a dispensing assembly comprising a reusable housing assembly and a cassette assembly having a reservoir prefilled with a fluid medicant. The dispensing assembly further comprises a pump assembly and a controller of the pump for pumping the medicant from the reservoir through a fluid path to an outlet for final infusion to a user. The method of use is to couple the cassette assembly to the reusable housing assembly, initiate an automatic prime function whereby the controller initiates a series of steps of operating the pump assembly and associated valve assemblies to prime the pump assembly and fluid path. In a further embodiment the dispensing assembly may include a volume sensor that is also primed by the automatic prime function. In a yet further embodiment, the cassette assembly may comprise an occluder assembly for isolating the reservoir from the fluid path until use, and the method further includes actuating the occluder from a closed state to an open state prior to initiating the automatic prime function. In a yet further embodiment, the cassette assembly is packaged for transport in a package body. The occluder assembly further comprises an occluder actuator, whereby removal of the actuator transitions the occluder assembly from the closed state to the open state. The occluder actuator is tethered to the package body by an occluder tether. The removal of the cassette assembly from the package body tensions the occluder tether and removes the occluder actuator from the actuator assembly thereby transitioning the occluder assembly from the closed state to the open state prior to coupling of the cassette assembly to the reusable housing assembly.

In accordance with another embodiment of the present disclosure, a drug delivery system may comprise a dispensing assembly comprising a reusable housing assembly and a cassette assembly having a reservoir prefilled with a fluid medicant. The dispensing assembly further comprises a pump assembly and a controller of the pump for pumping the medicant from the reservoir through a fluid path to an outlet for final infusion to a user. The method of use is to couple the cassette assembly to the reusable housing assembly, initiate an automatic prime function whereby the controller initiates a series of steps of operating the pump assembly and associated valve assemblies to prime the pump assembly and fluid path. The pump assembly may have an inlet valve assembly, a pump assembly and an outlet valve assembly. In some embodiments the outlet valve assembly can be fluidly between the pump assembly and a volume sensor assembly, and thereby the outlet valve assembly can also be a volume sensor valve assembly. The outlet valve assembly in order to allow the automatic prime function is biased to a closed position. Fluid pressure created by the pump assembly in the fluid channel greater than a set amount will open the outlet valve assembly and allow fluid to flow downstream to the volume sensor assembly. During the automatic prime function, the outlet valve assembly may be opened by a valve drive assembly to allow for automatic priming even when fluid pressure is below the set amount.

In accordance with another embodiment of the present disclosure, the cassette assembly of a dispensing system may be pre-filled and shipped to the end user. The cassette assembly includes an occluder assembly for isolating the prefilled reservoir from the pump portion of the cassette assembly. The occluder assembly includes a bung biasing the occluder assembly to the occlude state. The cassette assembly is shipped in a package for holding the cassette assembly, and further a tether connects the package to the occluder bung. Removing the cassette assembly from the package tensions the tether whereby the occluder bung is pulled from the occluder assembly and the occluder assembly transitions to the non-occluded state. The cassette assembly may then be coupled to a reusable housing assembly, and an automatic priming function initiated to prime the pump of the cassette assembly to prepare the dispensing system for infusion of fluid to the end user.

In accordance with another embodiment of the present disclosure, a bi-modal valve having a first mode the bi-modal is biased to a closed state and opens to allow fluid passage when fluid pressure on one side of the valve exceeds a predetermined threshold pressure. In a second mode of operation the bi-modal valve can be opened to allow fluid flow below the threshold pressure. The bi-modal valve has a valve actuator defining a valve face for sealing against a valve seat. The embodiment may further have a flexible diaphragm between the valve face and valve seat. The valve actuator further defines a valve lift slot for receiving a valve lift pin. A valve spring biases the valve actuator to a closed position wherein the valve face seals against the valve face. The valve lift slot and valve lift pin are configured so the slideably engages the slot where by the valve actuator can fully seat the valve face in the valve seat. In a first mode of operation, when fluid pressure on the inlet side of the valve overcomes the biasing force of the valve spring, the valve opens permitting fluid flow until the pressure is insufficient to maintain the valve in the open position and the valve closes. The opening of the valve moves the valve lift stop away from the valve lift pin. In the second mode, the valve lift pin is moved whereby the valve lift pin engages a valve lift stop at an end of the valve lift slot, overcomes the bias of the valve spring and opens the valve. Returning the valve lift pin to the original position allows the valve spring to return the valve to the closed position. The valve lift pin may be moved by a bell crank shaped drive arm rotated on a pivot by a shape memory actuator.

In accordance with another embodiment of the disclosure, a dual valve assembly is actuated by a common drive assembly having a first valve being bi-modal and a second valve having at least a single mode. The first valve having a first mode wherein the bi-modal is biased to a closed state and opens to allow fluid passage when fluid pressure on one side of the first valve exceeds a predetermined threshold pressure. In a second mode of operation the second valve can be opened by the drive assembly to allow fluid flow below the threshold pressure. The second valve can be opened and closed by the drive assembly independent of the first valve when the first valve in the first mode. When the first valve in the second mode, the second valve is also opened by the drive assembly. The first and second valve each have; a valve actuator defining a valve face and a valve slot with the valve slot having a lift slot stop at one end thereof, a valve seat for receiving the valve face to close the vale, and a valve spring to bias the valve to the close position. The drive assembly moves a lift pin for each valve, the lift pin for each valve arranged for sliding engagement with each respective valve lift slot. The valve drive may have a bell crank shaped lift moved by a shape memory actuator, the lift pins and lift slots arranged so that when the valve drive is in the first position, the first and second valve are closed, and whereby the first valve is in the first mode and can open when fluid pressure exceeds a threshold. In a second position, the valve drive engages the lift pin of the second valve against a valve lift stop of the second valve to open the second valve, and whereby the first valve continues to operate in the first mode whereby fluid pressure above the threshold opens the first valve. The drive mechanism having a third position whereby the second valve is opened by engagement of the lift pin of the second valve against the valve lift slot stop of the second valve, and the first valve is opened by the engagement of the lift pin of the first valve against the lift slot stop of the first valve.

### BRIEF DESCRIPTION OF THE DRAWINGS

These and other aspects will become more apparent from the following detailed description of the various embodiments of the present disclosure with reference to the drawings wherein:

FIG. 1 depicts a diagrammatic view of an exemplary drug delivery system;

FIG. 2 depicts a diagrammatic view of a fluid path within an example dispensing assembly.

FIGS. 3-8 depict diagrammatic view of a fluid path within an example dispensing assembly;

FIG. 9 depicts a perspective view of an example cassette assembly;

FIG. 10 depicts an exploded view of an example cassette assembly;

FIG. 11 depicts perspective view of an underside of an example cassette assembly with an example sealing wall exploded away;

FIG. 12 depicts a top down view of an example cassette assembly;

FIG. 13 depicts a cutaway view of an example cassette assembly, the cut being taken as indicated in FIG. 12;

FIG. 14 depicts a detailed view of the indicated region of FIG. 13;

FIG. 15 depicts a cross sectional view of an example cassette assembly taken at the cut plane indicated in FIG. 12;

FIG. 16 depicts a detailed view of the indicated region in FIG. 15;

FIG. 17 depicts a top down view of an example cassette base portion of an embodiment of a cassette assembly;

FIG. 18 depicts a perspective view of an example cassette base portion of an embodiment of a cassette assembly;

FIG. 19 depicts a cross section of an example cassette assembly taken at the cut plane indicated in FIG. 12;

FIG. 20 depicts a cross section of an example cassette assembly taken at the indicated cut plane in FIG. 12;

FIG. 21 depicts a detailed view of the indicated region of FIG. 20;

FIG. 22 depicts a cross section of an example cassette assembly taken at the indicated cut plane of FIG. 12;

FIG. 23 depicts a detailed view of the indicated region of FIG. 22;

FIG. 24 depicts a view of an example cassette assembly with a top cover portion of the cassette assembly exploded away from a cassette base portion of the cassette assembly;

FIG. 25 depicts a perspective view of an exemplary cassette assembly having a cassette top portion including a bay in which a cover is installed;

FIG. 26 depicts an exploded view of the exemplary cassette assembly shown in FIG. 25;

FIG. 27 depicts a bottom up view of an example cassette assembly with a sealing wall of the cassette assembly hidden;

FIG. 28 depicts an exploded view of an example cassette assembly;

FIG. 29 depicts a diagrammatic view of an example inspection system for a cassette assembly;

FIG. 30 shows a flowchart detailing a number of exemplary actions which may be executed to inspect a cassette assembly;

FIG. 31 depicts a cutaway view of an example cassette assembly including a cassette top portion with a bay into which a cover is coupled;

FIG. 32 depicts a perspective view of an exemplary cassette assembly having a cassette top portion including a bay in which a cover is installed;

FIG. 33 depicts an exploded view of a portion of an occluder assembly which may be included in a cassette assembly;

FIG. 34 depicts a perspective view of a cassette assembly in which an actuator of an occluder assembly has been removed;

FIG. 35 depicts a detailed view of the indicated region of FIG. 34;

FIG. 36 depicts a perspective view of an underside of a cassette top portion of an example cassette assembly having an actuator of an occluder assembly disposed in a port included in the cassette top portion;

FIG. 37 depicts a detailed view of the indicated region of FIG. 36;

FIG. 38 depicts a cross sectional view taken at the indicated cut plane in FIG. 27;

FIG. 39 depicts a detailed view of the indicated region of FIG. 38;

FIG. 40 depicts a top down view of an example embodiment of a cassette assembly;

FIG. 41 depicts a cross sectional view of an example cassette assembly taken at the indicated cut plane in FIG. 40;

FIG. 42 depicts a detailed view of the indicated region of FIG. 41;

FIG. 43 depicts a top down view of an example embodiment of a cassette assembly;

FIG. 44 depicts a cross sectional view of an example cassette taken at the indicated cut plane in FIG. 43;

FIG. 45 depicts a detailed view of the indicated region of FIG. 44;

FIG. 46 depicts a partially cut-away isometric view of the bung of FIG. 45.

FIG. 47 depicts an example cassette assembly and example filling implement;

FIG. 48 depicts a diagrammatic view of an example fill port and example interfacing portion of a filling implement;

FIG. 49 depicts a diagrammatic view of an example fill port and example interfacing portion of a filling implement;

FIG. 50 depicts a diagrammatic view of an example fill port and example interfacing portion of a filling implement;

FIG. 51 depicts a diagrammatic view of an example fill port and example interfacing portion of a filling implement;

FIG. 52 depicts a diagrammatic view of an example fill port and example interfacing portion of a filling implement;

FIG. 53 depicts a diagrammatic view of an example fill port and example interfacing portion of a filling implement;

FIG. 54 depicts a diagrammatic view of an example fill port and example interfacing portion of a filling implement;

FIG. 55 depicts a diagrammatic view of an example fill port and example interfacing portion of a filling implement;

FIG. 56 depicts a diagrammatic view of an example fill port and example interfacing portion of a filling implement;

FIG. 57 depicts a diagrammatic view of an example fill port and example interfacing portion of a filling implement;

FIGS. 58-60 depict diagrammatic views of an example reuse prevention assembly which may be included in a fill port of a cassette assembly;

FIGS. 61-62 depict diagrammatic views of an example fill port and example filling implement; and

FIG. 63 depicts a diagrammatic view of an example cassette assembly and example filling implement.

FIG. 64 depicts a perspective view of an example cassette assembly.

FIG. 65 depicts an exploded view of the example cassette assembly of FIG. 64.

FIG. 66 depicts a partial cutaway perspective view of a subassembly of the example cassette assembly of FIG. 64.

FIG. 67 depicts an enlarged view of a portion of the partial cutaway perspective view of FIG. 66 taken at 67.

FIG. 68 depicts a rotated perspective view of the example cassette assembly of FIG. 64.

FIG. 69 depicts an enlarged view of a portion of the example cassette assembly of FIG. 68 taken at 69.

FIG. 70 depicts a partial cutaway view of the example cassette assembly of FIG. 64.

FIG. 71 depicts an enlarged view of a portion of the example cassette assembly of FIG. 70 taken at 71.

FIG. 72A depicts another embodiment of the enlarged view of a portion of the example cassette assembly 70 taken at 71.

FIG. 72B depicts another embodiment of the enlarged view of a portion of the example cassette assembly of FIG. 70 taken at 71.

FIG. 73 depicts a perspective view of an occluder actuator.

FIG. 74 depicts a cross sectional view of the occluder actuator of FIG. 73.

FIG. 75 depicts a top perspective view of an occluder diaphragm.

FIG. 76 depicts a bottom perspective view of the occluder diaphragm of FIG. 75.

FIG. 77 depicts a top down view of the example cassette assembly of FIG. 64.

FIG. 78 depicts a cross sectional view of the example cassette assembly of FIG. 64 taken at B-B in FIG. 77 with an occluder actuator inserted.

FIG. 79 depicts an enlarged view of a portion of FIG. 78 taken at 79.

FIG. 80 depicts a top down view of the example cassette assembly of FIG. 64.

FIG. 81 depicts a cross sectional view of the example cassette assembly of FIG. 64 taken at B-B in FIG. 80 with an occluder actuator removed.

FIG. 82 depicts an enlarged view of a portion of FIG. 81 taken at 82.

FIG. 83 depicts a perspective view of the example cassette assembly of FIG. 77 with the occluder actuator inserted, with a cross sectional view at B-B.

FIG. 84 depicts enlarged view of the cross sectional view of FIG. 83 at D.

FIG. 85 depicts a perspective view of the example cassette assembly of FIG. 77 with the occluder actuator removed, with a cross sectional view at B-B.

FIG. 86 depicts an enlarged view of the cross sectional view of FIG. 85 at D.

FIG. 87 depicts a front view of a lift assembly for a measurement valve assembly and check valve assembly.

FIG. 88 depicts a perspective left side view of the lift assembly of FIG. 87.

FIG. 89 depicts a perspective right side view of the lift assembly of FIG. 87.

FIG. 90 depicts a top view of the lift assembly of FIG. 87 wherein the check valve assembly is open and the measurement valve assembly is closed.

FIG. 91 depicts a cross sectional view of the lift assembly and check valve assembly of FIG. 90 taken at A-A.

FIG. 92 depicts a cross sectional view of the lift assembly and measurement valve assembly of FIG. 90 taken at B-B.

FIG. 93 depicts a top view of the lift assembly of FIG. 87 wherein both the check valve assembly and measurement valve assembly are open.

FIG. 94 depicts a cross sectional view of the lift assembly and check valve assembly of FIG. 93 taken at A-A.

FIG. 95 depicts a cross sectional view of the lift assembly and measurement valve assembly of FIG. 93 taken at B-B.

FIG. 96 is a graphical representation power usage versus pump travel distance.

FIG. 97 is a perspective view of the cassette assembly of 64 in a cassette package.

### DETAILED DESCRIPTION

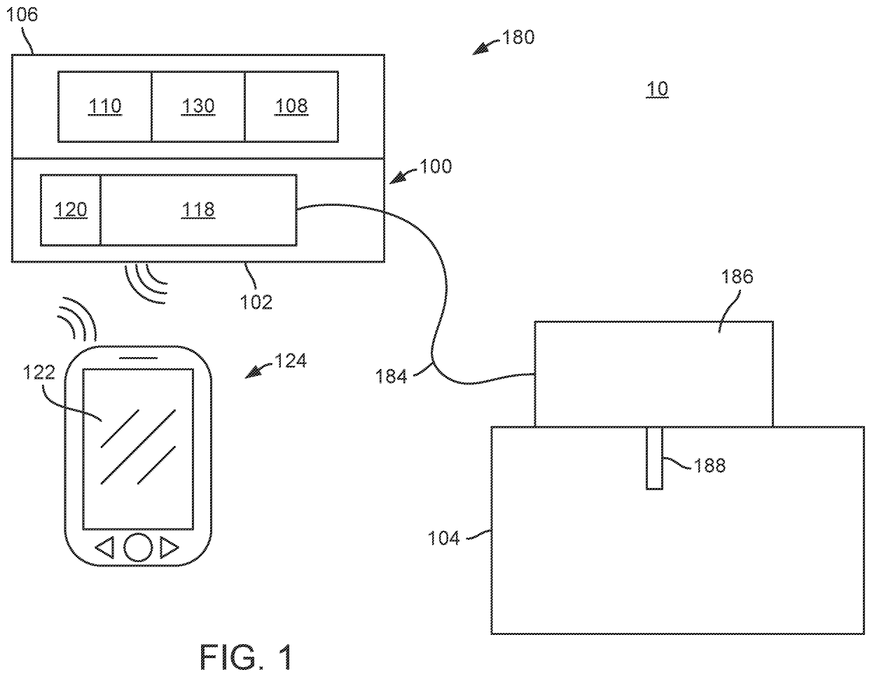

Referring to FIG. 1, an exemplary drug delivery system 10 is shown. The example drug delivery system 10 includes a dispensing assembly 100 which may be formed of a cassette assembly 102 and a reusable housing assembly 106. The dispensing assembly 100 may include a container or reservoir 118 and an access 120 (e.g. a pierceable member such as a septum). The cassette assembly 102 is coupled in use to the reusable housing assembly 106 having a controller 108 and a mechanical actuation assembly 110 which may be controlled to selectively dispense fluid from the dispensing assembly 100 (in some embodiments by acting on valving and pumping components included in the cassette assembly 102). As shown, the dispensing assembly 100 may be used in conjunction with an infusion device 186 of the drug delivery system 10. The infusion device 186 may be configured to be inserted into a patient to provide a fluid pathway from the reservoir assembly 100 into the patient 104 (e.g. to a subcutaneous layer of the patient 104's skin). To facilitate establishment of a fluid pathway into the patient 104's skin 104, the infusion device 186 may include a needle or cannula 188. The infusion device 186 may be fluidly connected to a length of tubing 184 and/or directly to the dispensing assembly 100. An outlet of the reservoir 118 may couple the dispensing assembly 100 directly or indirectly to the tubing 184 or infusion device 186 in certain embodiments. The dispensing assembly 100 may be controlled by a remote device 122 through wireless communication with the controller 108. The remote device 122 may be dedicated to communication with the dispensing assembly 100, or a more general device such as a cell phone or tablet running a specific application for the drug delivery system 10. The remote device 122 has a display for displaying for displaying information from the dispensing device 100, including status, warnings, alarms etc. The remote device 122 may also be used to enter information for relay to the dispensing device, programming infusion rates and initiating operations, functions, and modes of the dispensing assembly 100.

The various components described in relation to FIG. 1 may be, but are not limited to, those shown and described in one or more of the following: U.S. patent application Ser. No. 13/788,260, filed Mar. 7, 2013 and entitled Infusion Pump Assembly, now U.S. Publication No. US-2014-0107579, published Apr. 17, 2014 (Attorney Docket No. K40); U.S. Pat. No. 8,491,570, issued Jul. 23, 2013 and entitled Infusion Pump Assembly (Attorney Docket No. G75); U.S. Pat. No. 8,414,522, issued Apr. 9, 2013 and entitled Fluid Delivery Systems and Methods (Attorney Docket No. E70); U.S. Pat. No. 8,262,616, issued Sep. 11, 2012 and entitled Infusion Pump Assembly (Attorney Docket No. F51); U.S. Pat. No. 7,306,578, issued Dec. 11, 2007 and entitled Loading Mechanism for Infusion Pump (Attorney Docket No. C54); U.S. Provisional Application No. 62/597,246, filed Dec. 11, 2017 and entitled Infusion Pump Assembly (Attorney Docket No. P51); U.S Publication No. 2015/0281863, published Oct. 5, 2017 and entitled Infusion Set and Inserter Assembly (Attorney Docket No. U64); U.S. application Ser. No. 15/961,238, filed Apr. 24, 2018 and entitled Apparatus, System and Method for Fluid Delivery (Attorney Docket No. X37); U.S. Pat. No. 9,617,020, issued Apr. 11, 2017 and entitled Apparatus, System and Method for Fluid Delivery (Attorney Docket No. M60); and U.S. Provisional Application No. 62/809,248, filed Feb. 22, 2019 and entitled Infusion Set and Inserter Assembly Systems and Methods (Attorney Docket No. Y85), all of which are hereby incorporated herein by reference in their entireties. The systems and methods (including the cassette assemblies, reservoirs, filling aids, charging systems, volume sensing arrangements, control systems, inserter assemblies, etc.) described in any of the above-referenced applications and patents may also be used in conjunction with the various embodiments shown and described herein. The embodiments shown and described herein are not, however, limited to use therewith.

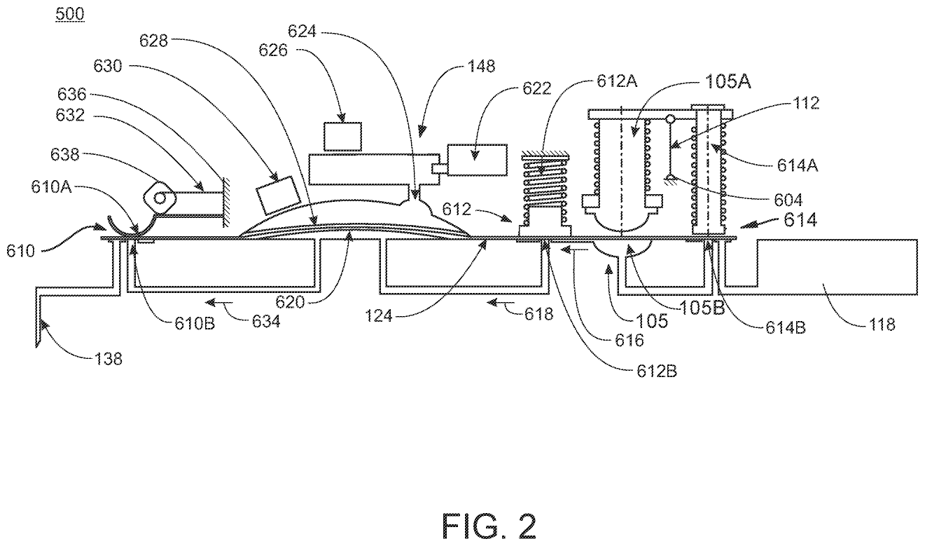

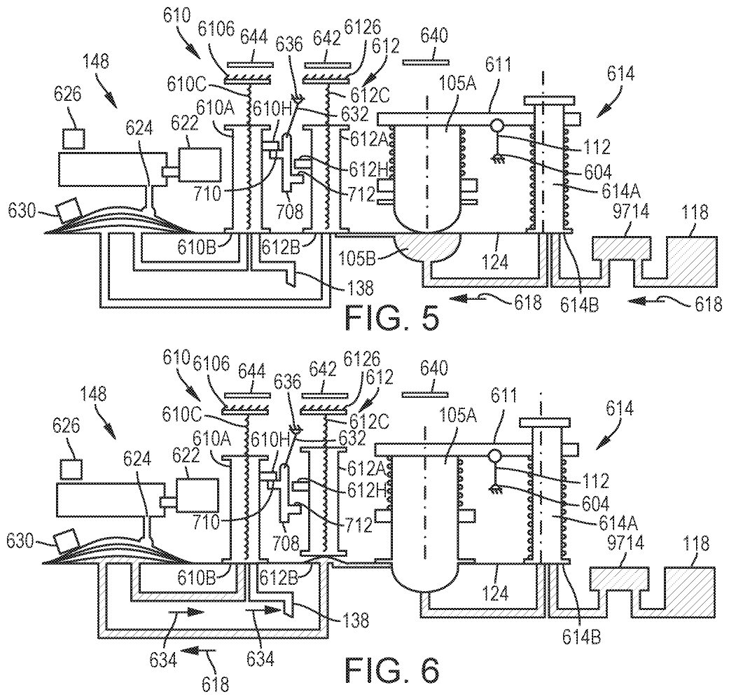

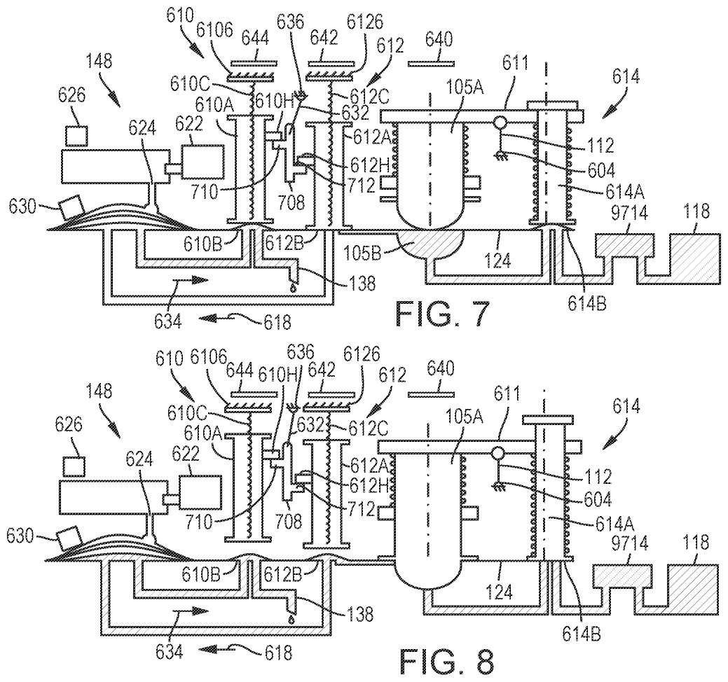

Referring now to FIG. 2-8, an exemplary dispensing assembly or arrangement 500 is shown. An occluder assembly 9714 isolates a filled reservoir 118 from the remainder of the dispensing assembly 500. Opening of the occluder assembly 9714 allows fluid to flow into the remainder of the dispensing assembly 500. In order to effectuate the delivery of fluid within the reservoir 118 to the user, the controller 108 included within the dispensing assembly 100 may command energizing of a shape memory actuator 112, which may be anchored on one end using a shape memory actuator anchor 604 and the other end to a common connector 611 attached to the pump 105 and inlet valve assembly 614. Energizing of the shape memory actuator 112 results in the activation of a pump 105 and a reservoir valve assembly 614. The reservoir valve assembly 614 may include a reservoir valve actuator 614A and a reservoir valve seat 614B. Activation of the reservoir valve assembly 614 may result in the downward displacement of the reservoir valve actuator 614A closing against the reservoir valve seat 614B, resulting in the effective isolation of the reservoir 118. The reservoir valve actuator 614A may press a membrane 124 included in the cassette assembly 102 against the reservoir valve seat 614B in order to close the reservoir valve assembly 614. Pump 105 and reservoir valve assembly 614 are arranged and connected by connector 611 whereby reservoir valve assembly 614 closes prior to pump 105 pumping fluid. Pump 105 may include a pump plunger 105A and a pump chamber 105B. The activation of the pump 105 may result in the pump plunger 105A being displaced in a downward fashion into the pump chamber 105B leading to a displacement of the fluid (in the direction of arrow 618, See FIG. 4). The membrane 124 may be included between the pump plunger 105A and the pump chamber 105B. The pump chamber 105B is shaped to be substantially the same as the end of the pump plunger 105A in order to substantially empty the pump chamber 105B with each stroke of the pump 105.

A check valve assembly or volume sensor valve assembly 612 may include a volume sensor valve actuator 612A and a volume sensor valve seat 612B. Referring also to FIG. 4, the volume sensor valve actuator 612A is maintained in a closed position via a volume valve spring assembly 612C, acting against a spring anchor 6126, that provides mechanical force to move the volume sensor valve actuator 612A against the volume sensor valve seat 612 B to seal volume sensor valve assembly 612. The volume sensor valve actuator 612A may press the membrane 124 included in the cassette assembly 102 against f the volume sensor valve seat 614B in order to close the volume sensor valve assembly 614. When the pump 105 is activated, however, if the displaced fluid is of sufficient pressure to overcome the mechanical sealing force of the volume sensor valve assembly 612, displacement of the fluid may occur in the direction of arrow 618. This may result in the filling of a volume sensor chamber 620 included within a volume sensor assembly 148. (See FIG. 6) Through the use of a speaker assembly 622, port assembly 624, reference microphone 626, spring diaphragm 628, and variable volume microphone 630, the volume sensor assembly 148 may determine the volume of fluid within the volume sensor chamber 620. Operation of such a volume sensor assembly 148 may be as discussed in, for example, U.S. Pat. No. 8,491,570 issued Jul. 23, 2013 and entitled Infusion Pump Assembly (Attorney Docket No. G75) which is incorporated herein by reference in its entirety above. Other suitable dispensed volume sensors may be used in other embodiments.

Referring also to FIG. 6, a shape memory actuator 632 may be anchored (on a first end) to a shape memory actuator anchor 636. Additionally, the other end of the shape memory actuator 632 may be used to provide mechanical energy to a bell crank lift arm 708, which may activate a measurement valve assembly 610. Once the volume of fluid included within the volume sensor chamber 620 is calculated, the shape memory actuator 632 is energized, resulting in the activation of measurement valve assembly 610 moving to the open position. The measurement valve assembly 610 may include a measurement valve actuator 610A and a measurement valve seat 610B. Once activated to lift the measurement valve actuator 610A from the measurement valve seat 610B due to the mechanical energy asserted on the fluid within volume sensor chamber 620 by the spring diaphragm 628, the fluid within the volume sensor chamber 620 may be displaced (in the direction of arrow 634) through cannula 188 and into the body of a patient 104. The measurement valve actuator 610A may then, by de-energizing the shape memory actuator 632 and by action of the measurement valve spring assembly 610C, acting against spring anchor 6106, press the membrane 124 included in the cassette assembly 102 against the measurement valve seat 610B in order to close the measurement valve assembly 610. In some embodiments, the membrane 124 included over the reservoir valve seat 614B, pump chamber 105B, volume sensor valve seat 612B, and the measurement valve seat 610B may be formed in a single piece of material having regions overlying each of these components.

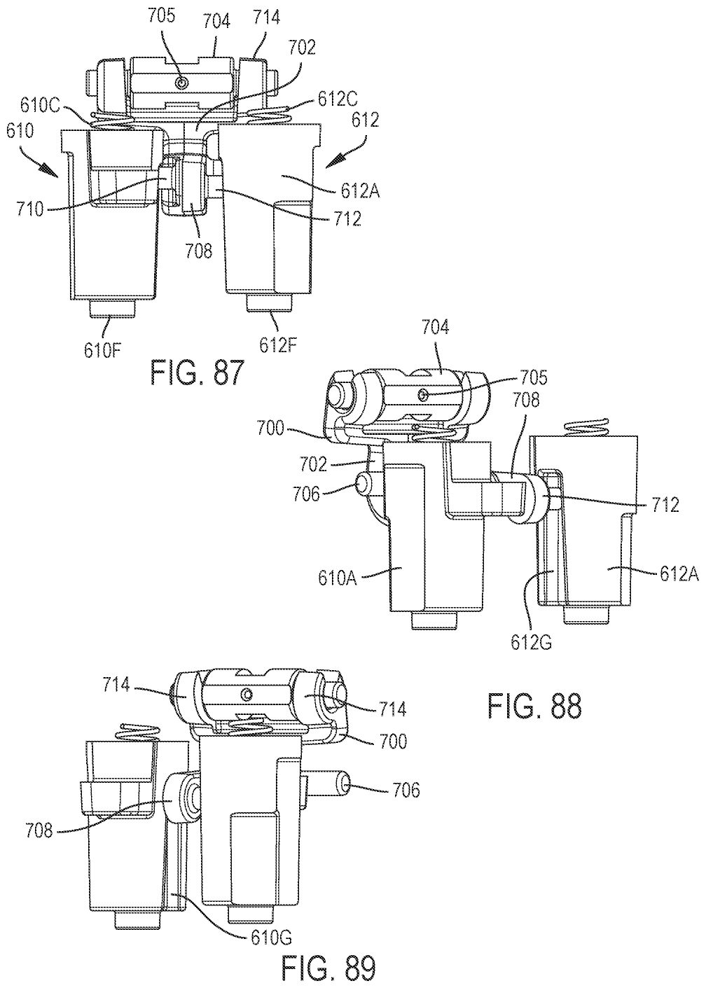

Referring now to FIGS. 87, 88 and 89, a drive assembly or bell crank assembly 638 has a substantially L shaped bell crank body 700 with a bell crank drive arm 702 for attachment to the shape memory actuator 632 by pivoting bell crank drive connector 704. The bell crank drive connector 704 is attached to the bell crank drive arm 702 by two pairs of bell crank connector support arms 714, each pair supporting one cylindrical end of the bell crank drive connector 704. The shape memory actuator 632 is affixed to the bell crank drive connector 704 through actuator attachment hole 705. In operation the pivoting bell crank drive connector allows for more linear motion and reduces bending forces of the shape memory actuator 632 as the shape memory actuator 632 pivots the bell crank body 700 on oppositely placed bell crank pivot pins 706. A bell crank lift arm 708, generally perpendicular to the bell crank drive arm 702, has opposite facing volume valve lift pin 712 and measurement valve lift pin 710. The volume valve lift pin 712 and measurement valve lift pin 710 are arranged to be generally parallel to the pivoting axis of bell crank pivot pins 706. The volume valve actuator 612A defines a hollow generally cylindrical cavity 612D to contain the volume valve spring assembly 612C. The volume valve spring assembly 612C expands between the bottom of the cavity 612D and the volume valve spring anchor 612G to hold the volume sensor valve assembly 612 in a default closed position wherein a volume valve face 612F of the volume valve actuator 612A is urged against the volume valve seat 612B to use the membrane 124 as a valve seal. In normal operation of the pump assembly for infusion of fluids from the dispensing assembly 100, the volume valve spring assembly 612C is calibrated that sufficient fluid pressure generated by the pump assembly 105 will overcome the spring force of the volume valve spring assembly 612C, allowing the volume valve actuator 162A to move away from the volume valve seat 612C and permit fluid flow.

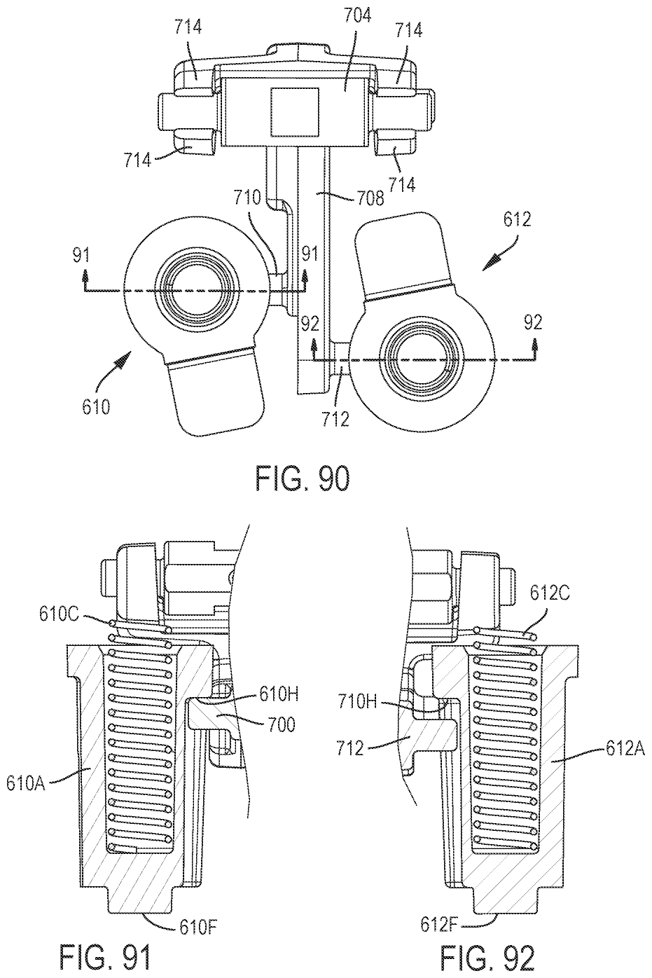

The measurement valve actuator 610A defines a hollow generally cylindrical cavity 610D to contain the measurement valve spring assembly 610C. The measurement valve spring assembly 610C expands between the bottom of the cavity 610D and the measurement valve spring anchor 610G to hold the measurement valve assembly 610 in a default closed position wherein a measurement valve face 610F of the measurement valve actuator 610A is urged against the measurement valve seat 610B to use the membrane 124 as a valve seal. In normal operation of the pump assembly for infusion of fluids from the dispensing assembly 100, the measurement valve spring assembly is calibrated that the fluid pressure in the volume sensor chamber 620 will be insufficient to overcome the spring force of the measurement valve spring assembly 610C. Measurement valve assembly 610 is opened by energizing the shape memory actuator 632 which then shortens in length, thereby applying a force to the bell crank assembly 638 through the bell crank drive connector 704, to rotate the bell crank body on the bell crank pivot pins 706. Rotation of the bell crank body 700 lifts the bell crank lift arm 708. The measurement valve actuator 610A defines a measurement valve lift slot 610G generally parallel to the measurement valve spring assembly 610C and the direction of the motion of the measurement valve actuator 610A in operation. The measurement valve lift pin 710 of the bell crank assembly 638 slideably engages in the measurement valve lift slot 610G. When the measurement valve assembly 610 is in the closed position, the measurement valve lift pin 710 rests at or below a measurement valve lift stop 610G defined at the end of the measurement valve lift slot 610G. Lifting the bell crank lift arm 708 engages the measurement valve lift pin 710 against the measurement valve lift stop 610G thereby causing the measurement valve actuator 610A to move away from the measurement valve seat 610C and permitting fluid flow. (See FIGS. 90, 91, 92) De-energizing the shape memory actuator 632 allows the measurement valve spring assembly 612C to return the measurement valve actuator 610A to the close position and to return the bell crank assembly 638 toward the rest position.

In the auto-priming mode of the dispensing assembly 100, it may be advantageous to mechanically open the volume sensing valve assembly 612 even when there is insufficient pressure in the fluid channel, the opening advantageous in order to purge air from the dispensing assembly 100 and to ensure the fluid channel is fully primed for infusion of fluid to the user. With particular reference to FIGS. 93, 94, 95, the volume valve actuator 612A defines a volume valve lift slot 612G generally parallel to the volume valve spring assembly 612C and generally parallel to the direction of the motion of the volume valve actuator 612A in operation. The volume valve lift pin 712 of the bell crank assembly 638 slideably engages in the volume valve lift slot 612G. When the volume sensor valve assembly 612 is in the closed position, the volume valve lift pin 712 rests at or below a volume valve lift stop 612G defined at the end of the volume valve lift slot 612G. Lifting the bell crank lift arm 708 engages the volume valve lift pin 712 against the volume valve lift stop 612G, thereby causing the volume valve actuator 612A to move away from the volume valve seat 612C and permitting fluid flow. De-energizing the shape memory actuator 632 allows the volume valve spring assembly 612C to return the volume valve actuator 612A to the close position and to return the bell crank assembly 638 toward the rest position.

The bell crank assembly 638 may have two modes, a normal pump operation mode that operates only the measurement valve assembly 610, and an auto-prime mode where the bell crank assembly operates both the measurement valve assembly 610 and the volume sensor valve assembly 612. In normal pump operation mode, the bell crank assembly 638 is pivoted to a first position by the shape memory actuator 632 whereby the measurement valve lift pin 710 engages the measurement valve lift stop 610H to lift the measurement valve actuator 610A before the volume valve lift pin 712 engages the volume valve lift stop 612H (See FIGS. 90, 92, 92). In the embodiment of FIGS. 90, 91, 92, the measurement and volume lift slot stops 610H and 612H are at substantially at the same elevation when the measurement valve assembly 610 and volume sensor valve assembly 612 are both in the closed position. The measurement valve lift pin 710 is offset from the volume valve lift pin 712 at a higher relative elevation whereby the measurement valve lift pin 710 opens the measurement valve assembly 610 while leaving the volume sensor valve assembly 612 in the closed position. Alternately, the measurement and volume lift slots 610H and 612H may be offset instead, or respective lift pins 710, 712 and lift stops 610H, 612H may both be offset to achieve the same outcome. In the auto-prime mode, the bell crank assembly 638 is rotated by the shape memory actuator 632 to a greater degree than the normal pump mode. In the auto-prime mode, the bell crank assembly 638 is rotated to the first position whereby the measurement valve lift pin 710 engages the measurement valve assembly 610 to move it to an open position, then continues to rotate to a second position whereby the volume valve lift pin 712 engages the measurement valve assembly to then open the volume valve lift assembly 612.

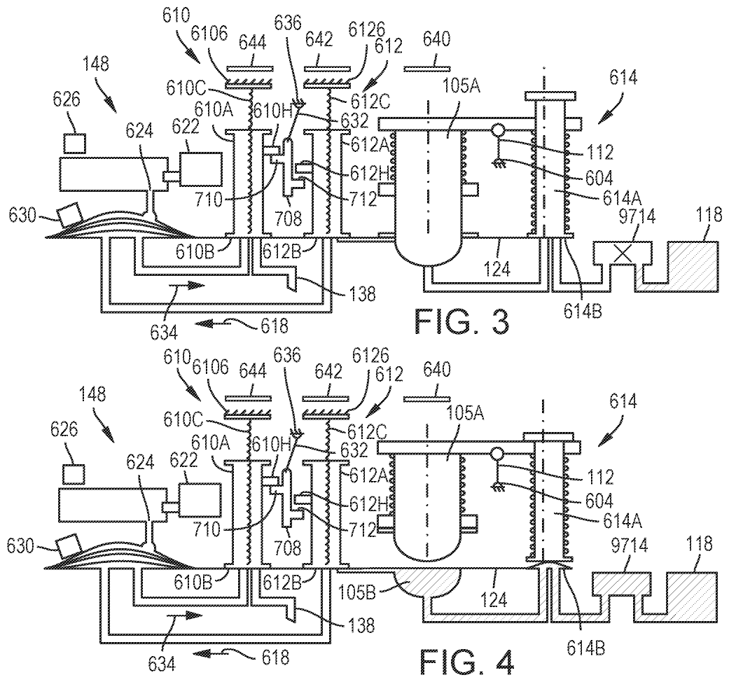

During the auto-priming operation, it is useful to sense the position of the pump plunger 105A, the measurement valve actuator 610A and the volume valve actuator 612A. There are various devices that may be used to sense the position of the pump plunger 105A, the measurement valve actuator 610A and the volume valve actuator 612A. These include, but are not limited to, one or more of the following: ultrasonic, optical (reflective, laser interferometer, camera, etc.), linear caliper, magnetic, mechanical contact switch, infrared might measurement, etc. However, in the exemplary embodiment, due to the small structure of the dispensing assembly 100 it is desirable to use a small component so as to utilize a small space with the sensing component(s). Sensing distance may also be a consideration in various embodiments. For example, where the displacement of the one or more components, e.g., the pump actuator 105A, measurement valve actuator 610A and volume valve actuator 612A, it may be very small (for example, in the exemplary embodiment, a full displacement of the pump actuator 105A may be about 1 mm and a full displacement of the measurement valve actuator 610A may be about 0.2 mm). A small reflective optical sensor assembly (hereinafter “optical sensor”) that fits into the exemplary embodiments of the reusable housing assembly 106, as shown and described, for example, herein, may be used. In some embodiments, the at least one optical sensor is located in the reusable housing assembly 106. The optical sensor, in the various embodiments, has a sensing range that accommodates the components for which the optical sensor may be sensing, e.g., the displacements of the pump actuator 105A, measurement valve actuator 610A and volume valve actuator 612A. In the exemplary embodiment any optical sensor may be used, including, but not limited to a Sharp GP2S60, manufactured by Sharp Electronics Corporation which is a U.S. subsidiary of Sharp Corporation of Osaka, Japan. In these embodiments, this optical sensor contains an infra red emitting diode and infra red sensing detector in a single component package. Light from the emitter is unfocused and bounces off the sensing surface, some of which is reflected to the detector. With reference to FIGS. 3-8, an optical pump sensor 640 is positioned to view the back of the pump actuator 105A, an optical measurement valve sensor 644 is positioned to view the back of the measurement valve actuator 610A, and an optical volume sensor 642 is positioned to view the back of the volume valve actuator 612A. Each optical sensor 640, 642 and 644 sensor senses light reflected from the back of the respective actuator 105A, 612A and 610A. The materials used in each actuator may be sufficiently reflective, for example with the use of white DERLIN for actuators 105A, 612A and 610A, for sensor operation. If there is insufficient optical reflection, an additional layer of higher reflective material can be added to the backs of actuators 105A, 612A and 610A to allow for reliable optical sensor operation. The optical sensors 640, 642 and 644 are used to sense the initial movement of, and position of, the respective actuators 105A, 612A and 610A.

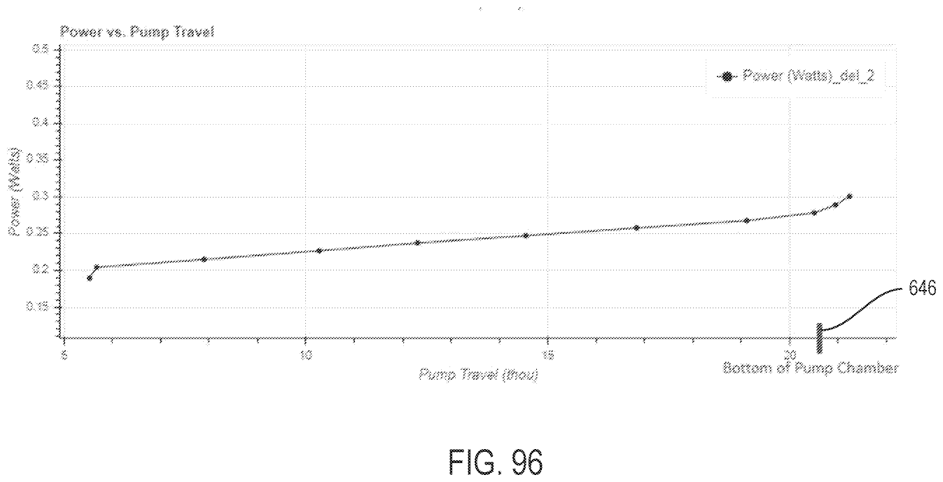

For auto-prime operation, it can be preferable for the pump actuator 105 to fully seat in the in the pump chamber 105B in order to remove all air from the pump chamber 105B. With reference to FIG. 96, the pump actuator 105A is controlled by the controller 108 by slowly incrementing the duty cycle of energy applied to the shape memory actuator 112. Each time the duty cycle is increased, it is held constant until the pump actuator stops moving as sensed by the pump sensor 640. For each position, power is calculated from the duty cycle and position is taken from the pump sensor 640 to add to a model of pump actuator position vs. power. The position vs. power model will remain linear as the pump actuator 105A moves through the pump chamber 105B, but will flatten as the pump actuator 105A hits the bottom of the pump chamber 105B. The stroke of the pump actuator 105A is stopped by the controller 108 when the model stops being linear as shown by line 646 of FIG. 96.

Since the pump actuator 105A moves fluid by displacement, the position of the pump actuator 105A sensed by pump sensor 640 may be correlated with the amount/volume of fluid displaced/pumped and could be used by the controller 108 to determine the volume of fluid pumped. For the measurement valve actuator 610A, measurement sensor 644 is used by the controller 108 to measure if the measurement actuator 610A is in the open or closed position. For the volume valve actuator 612A, volume sensor 642 is used by the controller 108 to measure if the measurement valve actuator 612A is in the open or closed position. Further, the position of pump actuator 105A, can be used by the controller 108 in controlling the energy applied to the shape memory actuator 112. The position of measurement of the measurement valve actuator 610A and volume valve actuator can be used by the controller 108 to control the amount of energy applied to the shape memory actuator 632.

In normal pumping operation, the measurement valve sensor 644 is used by the controller 108 to control the energy applied to the shape memory actuator 632. During the auto-priming operation, the volume valve sensor 642 is used to ensure that the volume valve actuator 612A is lifting and opening the volume valve assembly 612. The volume valve assembly 612 and the measurement valve assembly 610 are mechanically linked by the drive assembly or bell crank assembly 638, and that energizing the shape memory actuator 632 will start lifting the volume valve actuator 612A at some point. However it may not be known at what point in the stroke of the measurement valve actuator 610A the volume valve actuator 612A will start moving due to mechanical tolerance. For example, the volume valve actuator 612A could start moving when the measurement valve actuator 610A has been lifted by 0.015″, or it could start moving when the measurement valve actuator 610A has been lifted by 0.025″, which is a relatively large tolerance given that the check valve actuator 612A may open by lifting 0.005″. Therefore, it may not be known the position of the volume valve actuator 612A using only the measurement valve sensor 644. Therefor the volume valve sensor 642 is used by the controller 108 to control the shape memory actuator 632 when the opening of the volume valve actuator 612A is of interest.

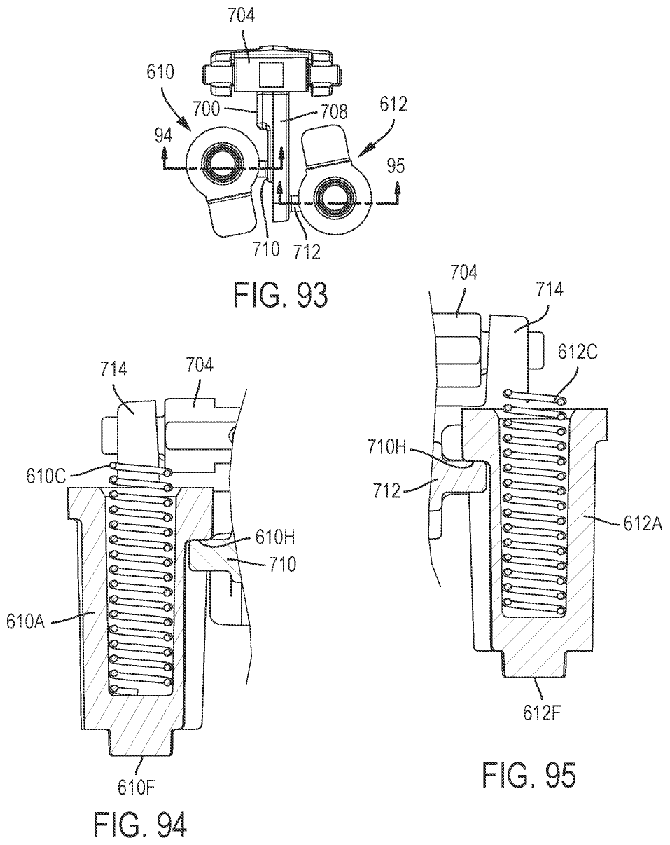

The auto-prime operation may be initiated by the user via the remote device by starting an auto-prime function. In one embodiment, the used will remove the pre-filled cassette assembly 102 from packaging (See below with relation to cassette package 8800). The cassette assembly 102 (or cassette assemblies 8500, 9500) is then engaged to the reusable housing assembly 106. The reusable housing assembly is then turned on and is capable of receiving instructions from the remote device 122. The user then may initiate an auto-prime function to prime the dispensing assembly 100 whereby air is purged from the fluid pathways and medicant is fills the fluid pathways to ready the dispensing assembly 100 for use. During the auto-prime function, the measurement valve assembly 610 and volume sensor valve assembly 612 are opened by the bell crank assembly 638. The pump assembly 105 and inlet valve assembly 614 are moved by the shape memory actuator 112. The inlet valve assembly 614 is first closed, then the pump assembly is actuated a full stroke to completely evacuate air from the pump chamber 105B. The controller 108 measures the power consumption of the shape memory actuator 112 for the length of travel of the pump actuator 105A. (See FIG. 94). Power consumption will be generally linear with pump travel over the length of the stroke of the pump actuator 105A. When the pump actuator bottoms out fully in the pump chamber 105B, the power consumption will increase in a non-linear amount relative to the prior travel. At bottom stroke, the membrane 124 will be fully pressed into the pump chamber 105B, evacuating the air therein. The volume sensor valve assembly 612 will next be allowed to close by the bell crank assembly 638. The pump actuator 105A is allowed to return to the initial position and the inlet valve assembly 614 is opened. The membrane 124 has a natural resilience and will withdraw from the pump chamber once the pump actuator 105A is withdrawn and the reservoir valve assembly 614 is opened. This return of the membrane 124 to the relaxed states creates a vacuum, drawing fluid from the reservoir 118. This series of actions is then repeated. First, open the measurement valve assembly 610 and volume sensor valve assembly 612, close the inlet valve assembly 614. Initiate a pump stroke of the pump actuator 105 of the pump assembly whereby the pump actuator 105A takes a full stroke to the bottom of the pump chamber 105B. Open the inlet valve assembly 614 and allow the pump assembly to return to the initial position. These steps are repeated until the user recognizes fluid being pumped from tubing 184. The user can than instruct the auto-prime function to cease by operation of the remote device 122. The auto-prime mode may have an additional step of counting the number of pump strokes and auto-stopping after completion of the predetermined number of strokes. This allows for auto-priming while not emptying the reservoir, even if the user fails to fails to notify the controller 108 of fluid exiting the tubing 184.

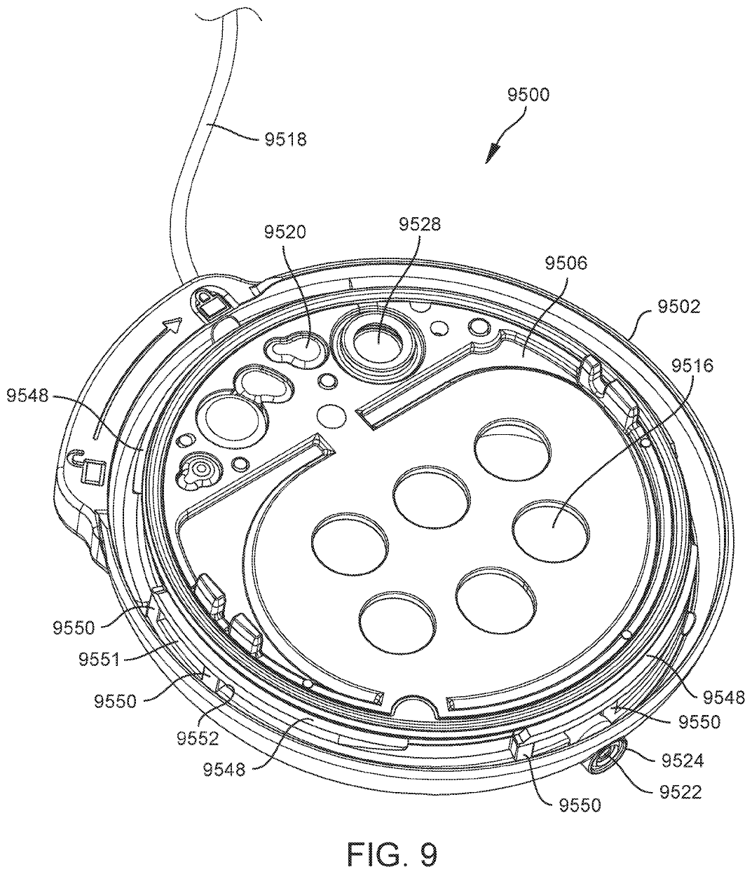

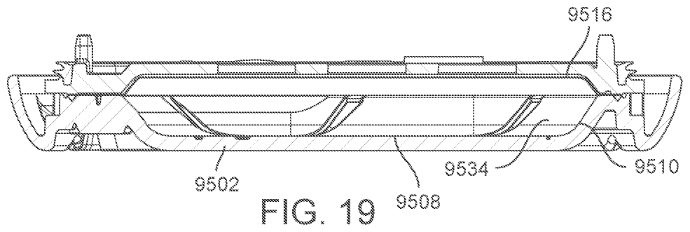

Referring now to FIGS. 9, 10, and 11, various views of an exemplary cassette assembly 9500 are depicted. The cassette assembly 9500 includes a cassette base portion 9502. The cassette base portion 9502 may include a reservoir recess 9508 which may be formed integrally therein. The cassette base portion 9502 of a prefilled cassette assembly 9500 may be formed from a long term drug compatible material such as a cyclic Olefin Polymer (COP), an example such as Zeonor® 1020R. Where the cassette assembly 9500 is user filled, the cassette base portion 9502 may be made of a Cyclic Olefin Copolymer (COC) such as Topas®, or of a polyester such as Tritan®. The reservoir recess 9508 may be covered by a piece of reservoir film 9516 which is coupled to the cassette base portion 9502. Together, the reservoir recess 9508 and reservoir film 9516 may define a reservoir 9536 (see, e.g. FIG. 19) for holding fluid (such as various drugs) in its interior volume. In certain embodiments, the fluid may be a drug for an endocrine disorder. For example, the fluid may be a diabetes management drug such as insulin. Short or rapid acting insulin (e.g. Aspart, Lispro, Glulisine, Velosulin, regular human insulin such as Novolin-R or Humulin R) may for example be used, though longer acting insulins (e.g. detemir, glargine, degludec, Toujeo) may be also be used. Cardiovascular drugs may also be used. For example, vasodilators or anti-hypertensive agents such as treprostinil may be used. Fluids may also include analgesics, chemotherapy drugs, enzymes, pegylated proteins, small molecules, natural products, peptide, proteins, nucleic acids, carbohydrates, nanoparticulate suspensions, and associated pharmaceutically acceptable carrier molecules.

The reservoir film 9516 may be affixed to the cassette base portion 9502 via adhesive, ultrasonic welding, heat sealing, etc. to generate a fluid tight seal between the cassette base portion 9502 and the reservoir film 9516. Alternatively, the reservoir film 9516 and cassette base portion 9502 may be compressively pinched together by a cassette top portion 9506 of the cassette assembly 9500 when the cassette top portion 9506 is coupled into the cassette assembly 9500. In some examples, and as described further later in the specification, the reservoir film 9516 may be affixed to the cassette base portion 9502 and the cassette top portion 9506 and the cassette top portion 9506 may be coupled in place in the cassette assembly 9500 by welding at least a portion of cassette top portion 9506 onto the reservoir film 9516. The cassette top portion 9506 may be made of the same material as the cassette base portion 9502 for improved welding together. The reservoir film 9516 may be constructed of a number of layers of materials which may be selected to add various desirable traits to the reservoir film 9516. Where applicable, tie layers may be used as well. In some embodiments, a drug compatible layer may form the interior volume facing surface of the reservoir film 9516, for example a long term drug compatible material such as a cyclic Olefin Polymer (COP), an example such as Zeonor® 1020R. A barrier layer which is impermeable to gas or specific gases may be included outward of the compatibility layer. The intermediate or tie layer may be an anethylene vinyl alcohol (EVOH). The outer layer may be polychlorotrifluoroethylene (PCTFE) such as Aclar®. Where layered film is used, the reservoir film 9516 may be a coextruded product. In alternative embodiments, the reservoir film 9516 may be constructed of nitrile, silicone, or chlorobuytl rubber.

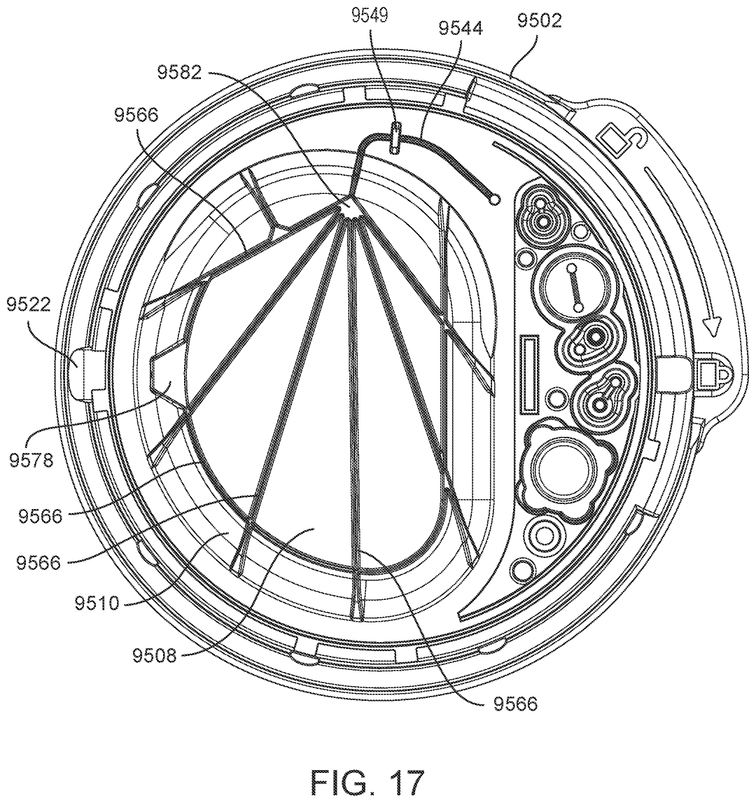

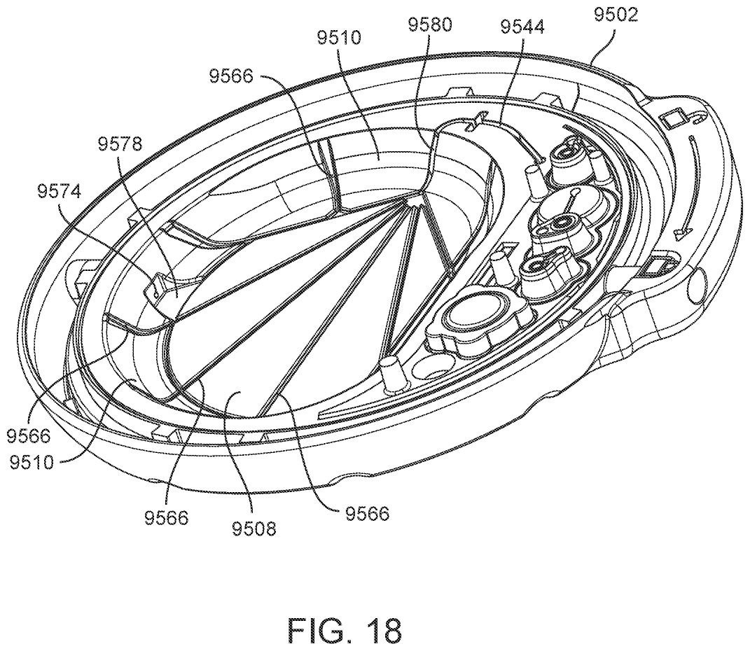

In the example embodiment, the reservoir film 9516 may include a preform region 9536 (best shown in FIG. 10). The preformed region 9534 may be vacuum or thermoformed. In the example embodiment, the preformed region 9534 is shown as a depression. The depression may be bowl like and may mimic the shape of the reservoir recess 9508 included in the cassette base portion 9502 of the cassette assembly 9500. When the reservoir film 9516 is coupled to the cassette assembly 9500, the preformed region 9534 may seat within the reservoir recess 9508 such that the reservoir film 9516 abuts the bottom surface of the reservoir recess 9508 and sits against the recess wall 9510. This may ensure that minimal air volume is present in the reservoir 9536 (see, e.g. FIG. 19) prior to filling of the reservoir 9536. In the example embodiment, the reservoir recess 9508 includes a variety of recessed ducts 9566 (described elsewhere herein, see, e.g., FIGS. 17-18). The preformed region 9534 may not include corresponding features. When the reservoir 9536 is in an empty state the reservoir film 9516 may not extend into the ducts 9566 thereby aiding in emptying of substantially all the fluid in the reservoir.

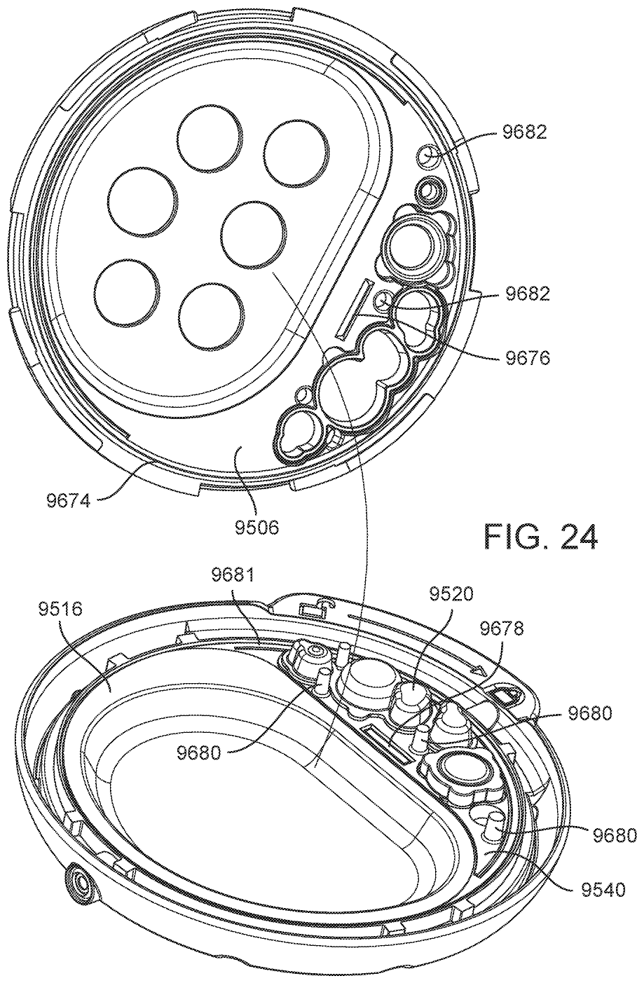

The reservoir film 9516 may include a peripheral rim region 9538. The peripheral rim region 9538 may be coupled to the cassette base portion 9502 at an attachment surface 9540 of the cassette base portion 9502 which may surround the reservoir recess 9508. In some examples, an outcrop 9542 may be included in the peripheral rim region 9538. Where the cassette top portion 9506 is welded onto the reservoir film 9516, the cassette top portion 9506 may include an energy director 9674 (see, e.g. FIG. 24) which aligns over the peripheral rim region 9538 when in place within the cassette assembly 9500. Thus, the cassette top portion 9506 may be welded onto the top surface of the peripheral rim region 9538 of the reservoir film 9516 to retain the cassette top portion 9506 in place within the cassette assembly 9500.

As shown, in certain examples, the attachment surface 9450 may include one or more features which are recessed therein. When coupled in place within the cassette assembly 9500, the reservoir film 9516 may form a seal over these features. In the example embodiment, a reservoir outlet path 9544 is recessed into the attachment surface 9540. An air trap housing 9546 disposed along the reservoir outlet path 9544 is also present and is shown as a recessed feature in the attachment surface 9540 in the example embodiment. The air trap housing 9546 may be a trench which is oriented at an angle with respect to a portion of the reservoir outlet path 9544. In the example embodiment, the air trap housing 9546 is substantially perpendicular to the reservoir outlet path 9544. In some embodiments, an air trap 9547 may be placed within the air trap housing 9546. The air trap 9547 may in some embodiments be a piece of mesh or screen 9549 placed in the air trap housing 9546 to help entrain air bubbles and prevent them from proceeding downstream. The outcrop 9542 of the reservoir film 9516 may seal over the reservoir outlet path 9544 and air trap 9547 within the air trap housing 9546 to form an enclosed and leak tight fluid pathway out of the reservoir 9536 (see, e.g. FIG. 19).

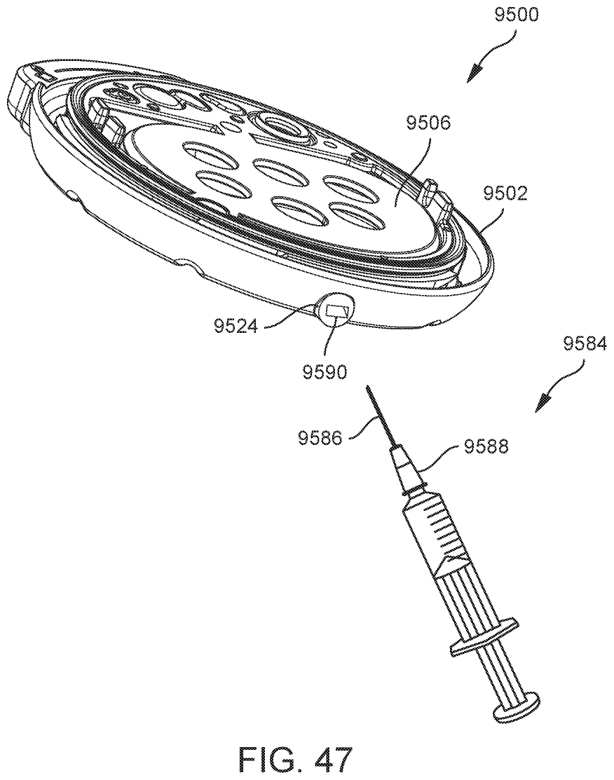

A fill port 9524 may also be included in the cassette base portion 9502. In the example embodiment, the fill port 9542 extends through a side wall of the cassette base portion 9502. The fill port 9524 may extend directly to the reservoir 9536 (see, e.g. FIG. 19) and may be plugged with a septum 9522. The septum 9522 may be pierceable via a sharp 9586 (see, e.g., FIG. 47) on a filling implement 9584 (see, e.g. FIG. 47) and self-seal upon removal of the sharp 9586 so as to fluidically seal the reservoir 9536 once the reservoir 9536 has been loaded. As described elsewhere herein, the fill port 9524, filling implement 9584, or a combination of the two may be configured to prevent reuse of the reservoir 9536 once a filling operation has been performed. Also as described elsewhere herein, the fill port 9524, filling implement 9584, or a combination of the two may also be configured to ensure access to the reservoir 9536 is restricted to only appropriate filling implements 9584. Other portions of a cassette assembly 9500 such as the cassette top portion 9506 and cassette base portion 9502 may include features for these purposes. In such embodiments, these features may cooperate with filling implements 9584 or work in conjunction with the fill port 9524 to inhibit unintended access. The fill port 9524 in the example embodiment is disposed opposite the tubing 9518 extending from the cassette base portion 9502.

In the example embodiment, the cassette assembly 9500 is arranged to mate with a reusable housing assembly 106 via a twist lock type engagement and includes part of a rotational coupling to facilitate this. Other types of coupling arrangements are possible in other embodiments. In the example embodiment, the cassette top portion 9506 of the cassette assembly 9500 includes a number of tabs 9548 (see, e.g. FIG. 9) which extend radially from the sides of the cassette top portion 9506. In the example embodiment, the tabs 9548 are evenly spaced at regular angular intervals, however, in other embodiments, the spacing may differ. The cassette base portion 9502 may include stop surfaces 9550 which prevent the reusable housing 106 from being twisted too far or in the wrong direction when coupling the cassette 9500 and the reusable housing 106. The tabs 9548 and the stop surfaces 9550 may cooperate to form mating tracks. The stop surfaces 9550 may be disposed substantially perpendicularly to the tabs 9548 as shown. The tabs 9548 projecting from the cassette top portion may overhang a peripheral recess included in the cassette base portion 9502 adjacent the side wall of the cassette base portion 9502. The stop surfaces 9550 may project into the peripheral recess.

During coupling, a coupling tab or finger of the reusable housing 106 may be passed through an opening 9551 between a stop surface 9550 and a tab 9548 and rotated under the tab 9548 along a twist path until reaching the appropriate stop surface 9550. The undersurface of each tab 9548 may include a ramped portion 9554 which may cause an environmental sealing ring 9556 (which may be formed of an overmolded elastomer) included on the cassette top portion 9506 to be compressed against the reusable housing assembly 106 (so as to form a fluid seal) during coupling together the cassette assembly 9500 and reusable housing assembly 106. Each tab 9548 may include a detent region 9552 into which the coupling tab on the reusable housing assembly 106 may seat once the stop surface 9550 has been reached. When in the detent region 9552, the coupling tab of the reusable housing 106 may resist rotation unless a downward force is applied during the twisting motion.

The fill port 9524 in the example embodiment is disposed in a non-traversed region outside of the twist path of any of the coupling tabs of the reusable housing assembly 106. The fill port 9524 may, for instance, be placed between a non-traversed region between two of the stop surfaces 9550. This may allow the fill port 9524 to be placed in a side wall of the cassette base portion 9502 while still allowing for a twist lock type engagement between a cassette assembly 9500 and reusable housing assembly 106.

The cassette assembly 9500 may further include a valve membrane cover 9520. The valve membrane cover 9520 (see, e.g. FIG. 10) may include a number of regions which seat over a pumping chamber recess 9532 and valve features 9514 (such as those described above in FIGS. 2-8) included in the cassette base portion 9502. In the example, the valve features 9514 are shown as volcano type valves. The valve membrane cover 9520 may be constructed of a flexible material. Select regions of the valve membrane cover 9520 may be actuated toward and away from the valve features 9514 and pumping chamber recess 9532 via pumping and valve actuating components contained in a reusable housing 106 (such as those described above in relation to FIGS. 2-8). This may direct and control the flow of fluid being transferred from the reservoir 9536 (see, e.g. FIG. 19) toward a patient 104. The valve membrane cover 9520 may be captured between the cassette base portion 9502 and cassette top portion 9506 during assembly of the cassette assembly 9500. In some embodiments, the valve membrane cover 9520 may be overmolded onto the cassette base portion 9502, compressively pinched between the cassette base portion 9502 and cassette top portion 9506, or affixed in place via adhesive, heat sealing, or another suitable process. A fluid tight seal may be formed between the cassette base portion 9502 and the valve membrane cover 9520 when the cassette assembly 9500 is assembled.

A volume sensor diaphragm assembly 9526 (see, e.g., FIG. 10) including a diaphragm 9528 and frame 9530 may also be coupled into the cassette assembly 9500 between the cassette base portion 9502 and the cassette top portion 9506. The volume sensor diaphragm assembly 9526 may be placed on a volume sensing station 9564 of the cassette base portion 9502. This may form a volume sensing chamber which may be monitored to determine volumes of fluid being transferred through the cassette assembly 9500. Further description is provided above in relation to FIG. 4.

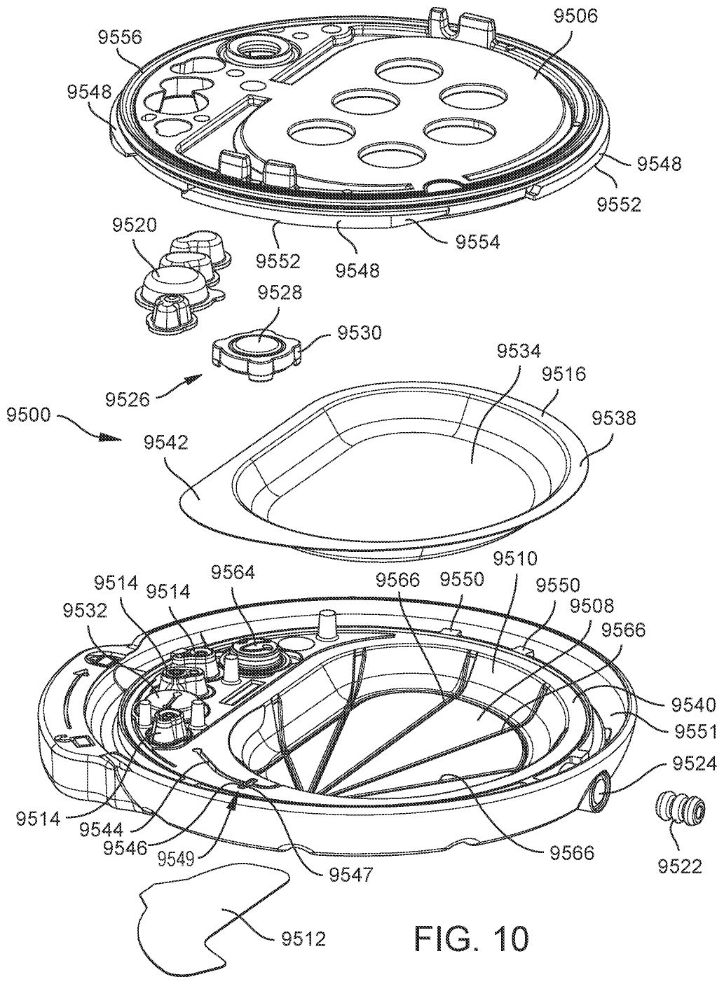

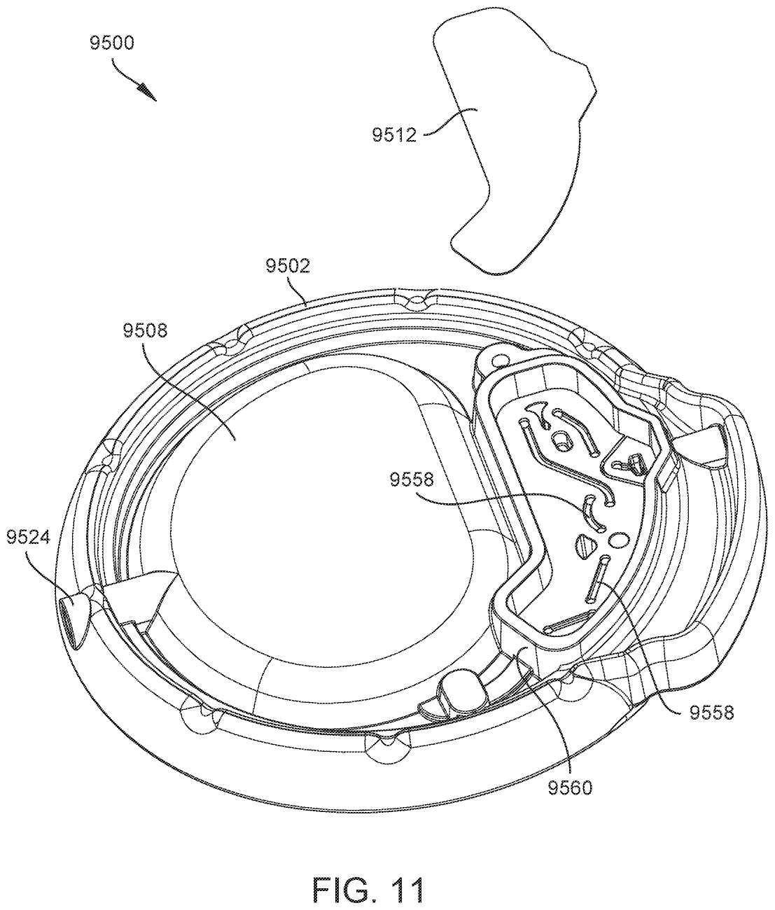

The flow pathways leading into and out of the valve features 9514, pumping recess 9532, and volume sensing chamber may extend to flow channels 9558 (best shown in FIG. 11) defined in the opposing side of the cassette base portion 9502. These flow channels 9558 may allow the valve features 9514 and pumping chamber recess 9532 to fluidically communicate with one another. As fluid is pumped from the reservoir 9536 toward the patient 104, the fluid may be transferred and routed through these flow channels 9558. Interspersed among the flow channels 9558, additional recesses 9559 may be present. These recesses 9559 may help to allow cassette base portions 9502 to be molded with a shorter mold cycle time and may help to prevent any distortion of the flow channels 9558 during cooling.

A film sheet seal 9512 may be coupled onto the cassette base portion 9502 over the flow channels 9558 so as to form a seal over these flow channels 9558 which keeps fluid therein within the confines of the flow channels 9558. The film sheet seal 9512 may be attached to the cassette base portion 9502 in any suitable manner. In certain embodiments, the film sheet seal 9512 may be coupled to the cassette base portion 9502 via heat sealing. The film sheet seal 9512 may be constructed of the same material used to create the reservoir film 9516 and may be a flexible material. In the example embodiment, the flow channels 9558 are included within a well 9562 defined by a surrounding wall or rim 9560 included in the cassette base portion 9502. The rim 9560 may help protect the film sheet seal 9512 and may provide an alignment aid which may be used to locate the film sheet seal 9512 during manufacture of a cassette assembly 9500. In alternative embodiments, a rim 9560 may not be included and other alignment aids such as alignment pins or projections may be utilized.

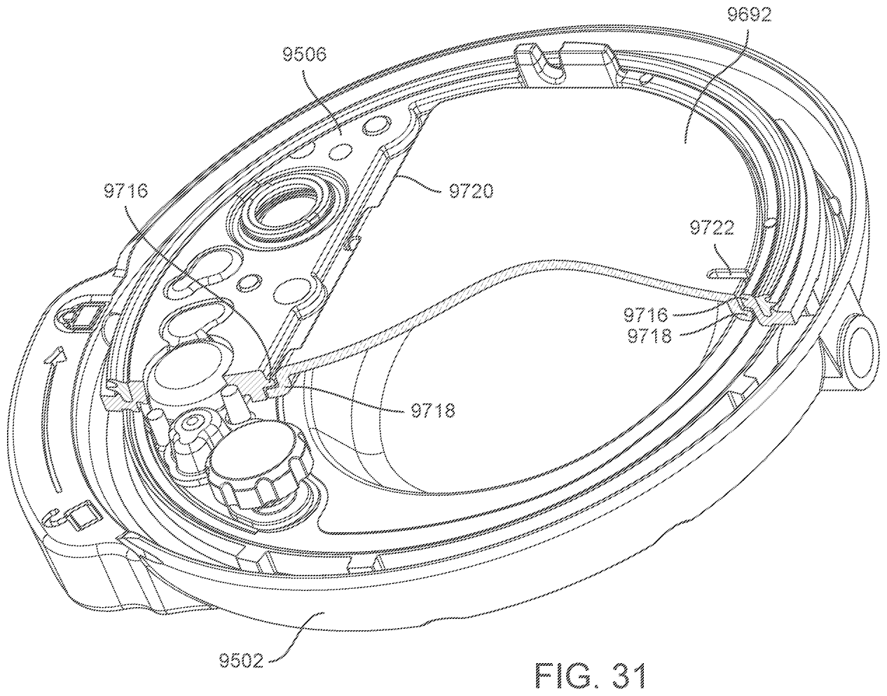

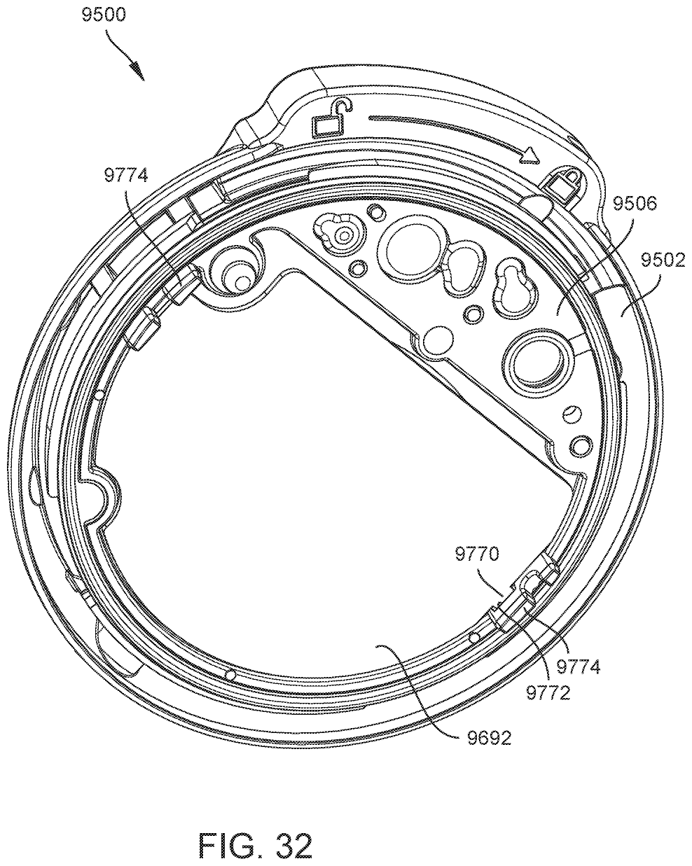

In some embodiments, a cover (not shown) may be included and may couple in place above the film sheet seal 9512. This cover may couple in place via snap fit, interference fit, a welding process, adhesive, solvent bonding, etc. The cover may be constructed of a rigid or puncture resistant material and may serve as a protector for the film sheet seal 9512. Preferably, the cover may couple to the cassette base portion 9502 in a manner which makes the cover difficult to remove by a user. This cover may be coupled to the cassette base portion 9502 similarly to the manner in which cover 9692 described later in the specification (see, e.g. FIGS. 31-32) may be coupled in place over the reservoir 9536. In some examples, a cover may not be used, however, the film sheet seal 9512 may be constructed of thicker film than that used to construct the reservoir film 9516. The film sheet seal 9512 may also include one or more additional layer that adds durability to the film sheet seal material 9512.

In alternative embodiments, a film sheet seal 9512 may not be used. Instead, another suitable sealing wall such as a sealing plate 9774 (see, e.g. FIG. 44) may be used. A sealing plate 9774 may be laser welded over the flow channels 9558 to seal over the flow channels 9558. In some examples where a plate 9774 is used, the flow channels 9558 may be defined in the plate 9774 as opposed to the cassette base portion 9502. It may, however, be advantageous to use a film sheet seal 9512 depending on the embodiment. For example, where a plate 9774 is laser welded to the cassette base portion 9502 to seal the flow channels 9558, the plate 9774 may have tight flatness tolerances (e.g. not more than one thousandth of an inch variation from flat). As a film sheet seal 9512 is flexible, these tolerancing issues may be abated. With wider allowable tolerancing, more flexibility in molding may be gained as well.

Additionally, various embodiments may find it advantageous to include the flow channels 9558 in the cassette base portion 9502 as opposed to a plate 9774 (see, e.g. FIG. 44) whether or not a film sheet seal 9512 or plate is used. Since the flow channels 9558 may be molded with the rest of the cassette base portion 9502, it may be ensured that flow pathways leading into and out of the valve features 9514, pumping recess 9532, and volume sensing chamber, etc., align with the flow channels 9558. Thus, use of a film sheet seal 9512 may simplify manufacturing of a cassette assembly 9500 and may limit the number of different materials and manufacturing processes used in the production of the cassette assembly 9500. In turn, this may help to decrease compatibility testing burden on a cassette assembly 9500.

Use of a film sheet seal 9512 may also allow for the flow channels 9558 to be made smaller or arranged closer together in a denser layout. This may facilitate a more compact cassette assembly 9500 or the saved space may be used to allow a cassette assembly 9500 with a larger reservoir 9536 to be made without increasing the overall footprint of the cassette assembly 9500. For example, where flow channels 9558 are included in a cassette base portion 9502 and sealed by a laser welded plate 9774, the layout of the flow channels 9558 may need to be planned so as to accommodate melting of the cassette base portion material 9502 during the laser weld. During welding, the laser may pass through the sealing plate 9774 and the laser's energy may be absorbed at the surface of the cassette base portion 9502. Thus, the heating which occurs may be predominantly in the cassette base portion 9502 in the area of the flow channels 9558. Sufficient space may need to be included around the flow channels 9558 to ensure that melting of the cassette base portion 9502 does not distort, close, or otherwise obstruct the flow channels 9558. Attaching a film sheet seal 9512 via a heat sealing process, however, may minimize melting of cassette base portion 9502. Heating during in this process may be shifted away from the cassette base portion 9502 and instead may be applied from an external source through the film sheet seal 9512 material. Thus, the film sheet seal 9512 may be coupled to the cassette base portion 9502 with minimal melting of the cassette base portion 9502.

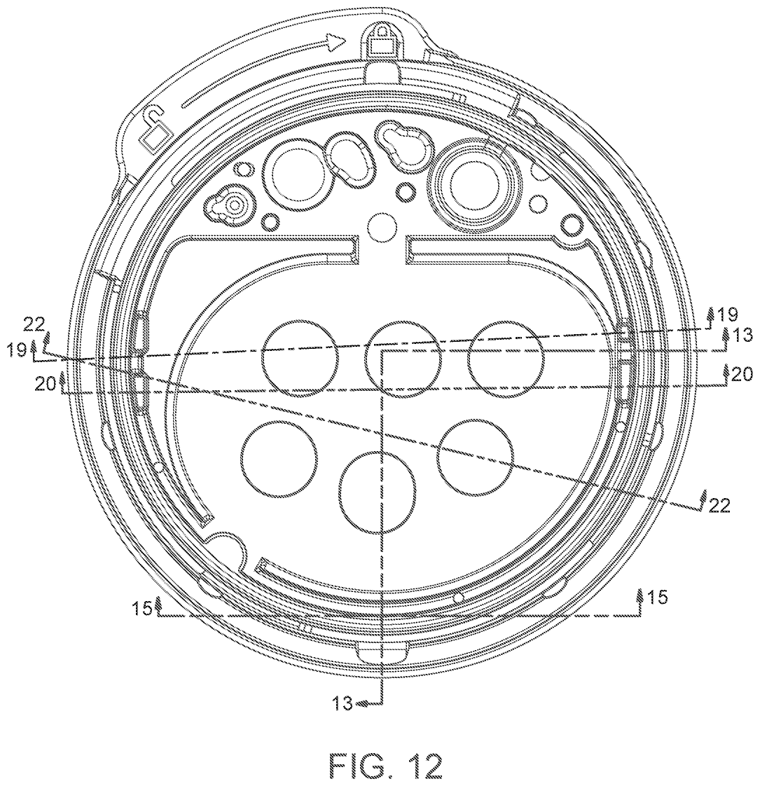

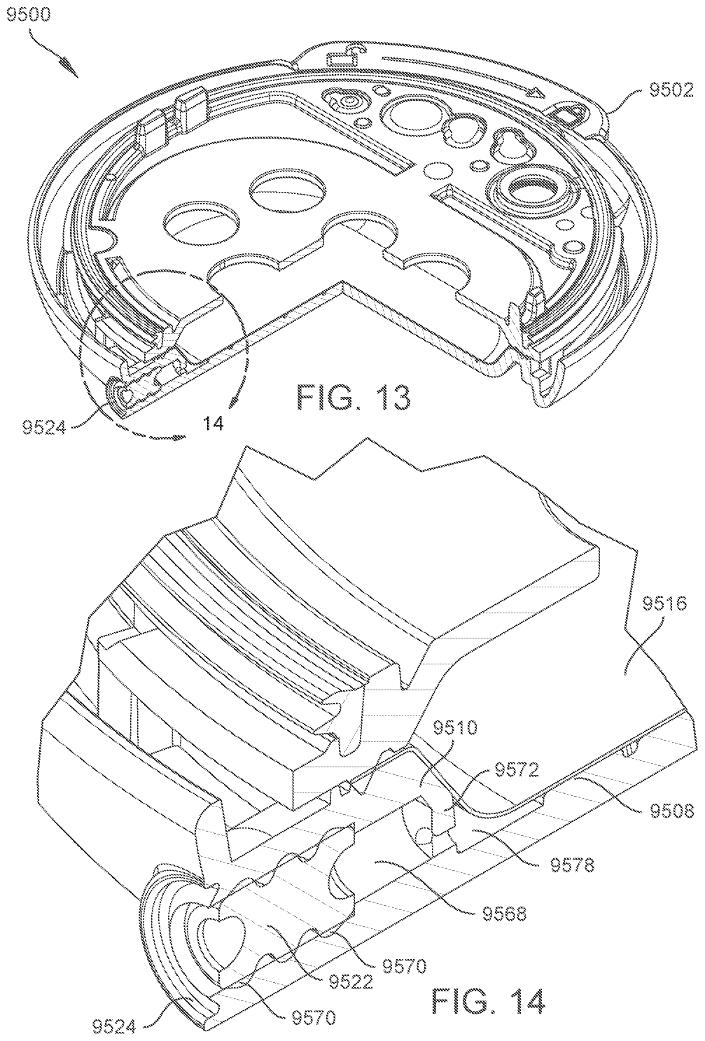

Referring now to FIG. 13 (a perspective three quarter view which is cut as indicated in FIG. 12) and FIG. 14 (a detailed view of region 14 in FIG. 13), the fill port 9524 may provide a direct pathway into the interior volume of the reservoir 9536. As shown, the fill port 9524 may have a bore 9568 within which the septum 9522 may be placed. The septum 9522 may include one or more ribs 9570. The ribs 9570 may have a diameter (at their widest point) that is somewhat larger (e.g. 140-105%) than the diameter of the bore 9568. The fill port 9524 may also include a guard wall 9572. The guard wall 9572 may be disposed at the interior end of the bore 9568. The guard wall 9572 may block a user from extending a sharp 9586 (e.g. a needle) past a certain point. This may protect the reservoir film 9516 from being damaged by the sharp 9586 (see, e.g., FIG. 47) when the reservoir 9536 is filled.

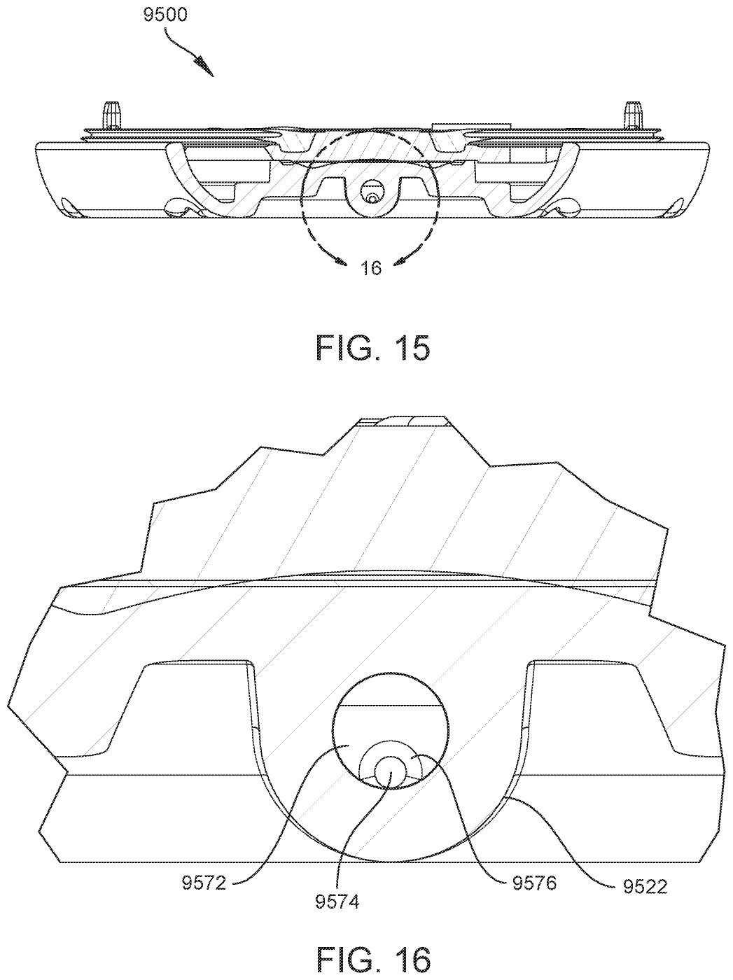

Referring now also to FIG. 15 (a cross sectional view taken at the indicated cut plane in FIG. 12) and FIG. 16 (a detailed view of region 16 in FIG. 15), the guard wall 9572 may include the inlet 9574 to the reservoir 9536. A funnel region 9576 included in the guard wall 9572 may surround the inlet 9574. The funnel region 9576 may help to redirect any sharp 9586 long enough to reach the guard wall 9572 toward the inlet 9574. As shown, the reservoir recess 9508 may include a basin 9578 which is located downstream of the inlet 9754. The basin 9578 may be recessed deeper than the surrounding portion of the reservoir recess 9508. As best shown in FIG. 14, due to the preforming the reservoir film 9516 may be positioned above the bottom surface of the basin 9578 when the reservoir 9536 is in an empty state. Thus, the basin 9758 may provide an open space where a sharp 9586 may project without contacting the reservoir film 9536.

In some embodiments, the fill port 9524 may be arranged such that the filling implement 9584 (see, e.g., FIG. 47) for the reservoir 9536 includes a sharp 9586 (see, e.g., FIG. 47) which is not long enough to reach into the interior volume of the reservoir 9536. In such embodiments, the sharp 9586 may be extended through the septum 9522 and into a receiving volume in the bore 9568 of the fill port 9522 which is upstream of the guard wall 9572. Fluid may be delivered into the receiving volume and flow into the reservoir 9536 through the inlet 9574 in the guard wall 9572. This may inhibit a user from contacting the reservoir film 9516 with the sharp 9586 during a filling operation. Preferably, any receiving volume including in the fill port 9522 may be relatively small so as to minimize any dead space which may retain fluid as the reservoir 9536 is emptied.

In some embodiments, and as described later in the specification, the fill port 9524 may include one or more features which may restrict ability to access the interior volume of the reservoir 9536. For example, the fill port 9524 may include one or more feature which inhibits access to the interior volume for filling implements 9584 (see, e.g., FIG. 47) unintended for use with the cassette assembly 9500. Additionally or alternatively, and again as described later in the specification, the fill port 9524 may include one or more feature which may prevent or limit reuse of the cassette assembly 9500. The fill port 9524 may, for example, include one or more feature which may block or make accessing the interior volume of the reservoir 9536 more difficult. This may help to ensure that the cassette assembly 9500 is not used for more than its intended usage life (in some embodiments, this may be a single use).

In some embodiments or for particular applications, multiple cassette assembly 9500 types may be produced. In some embodiments, a drug for use with the cassette assembly 9500 may be available in different concentrations. Using insulin as an example, cassette assemblies for U100 insulin and U200 insulin may be available. Filling implements 9584 containing U200 insulin may include a sharp 9586 which is of a length insufficient to pierce through the septum 9522 of a U100 cassette assembly 9500. This may prevent a user from accidentally filling the reservoir 9536 with a stronger concentration drug than intended.

Referring now to FIGS. 17 and 18, as mentioned above, the reservoir recess 9508 may include a number of recessed ducts 9566. These ducts 9566 may aid in preventing pocketing of fluid within the collapsible reservoir 9536 as the reservoir 9536 is depleted and may help to facilitate complete emptying of the reservoir 9536. All of the ducts 9566 may extend into communication with the reservoir outlet path 9544 of the cassette base portion 9502. In the example embodiment, an outlet duct 9580 which is recessed into the reservoir recess wall 9510 leads into the reservoir outlet path 9544. A confluence region 9582 may be present at the upstream end of the outlet duct 9580. A number of ducts 9566 may furcate from the confluence region 9582 to various portions of the reservoir recess 9508. In the example embodiment, five ducts 9566 extend from the confluence region 9582 and are spaced at regular angular intervals. These ducts extend in a substantially straight line path across the reservoir recess 9508 and up the recess wall 9510. Each, some, or at least one of these ducts 9566 may further furcate into additional ducts 9566 which form tributaries for the parent duct 9566. Ducts 9566 which extend along a curved or at least partially curved path are also possible. In the example embodiment, a duct 9566 which is recessed around a majority of the perimeter of the reservoir recess 9508 adjacent the recess wall 9510 is included. This duct 9566 may cross the paths of other ducts 9566 included in the cassette base portion 9502 and may also extend to the basin region 9578 included by the inlet 9574 to the reservoir 9536.

The cross sectional area of the flow paths created by the recessed ducts 9566 may be constant or may change along at least a portion of the duct 9566. In the example embodiment, the recessed ducts 9566 increase in width as they extend up the recess wall 9510. The depth of the ducts 9566 may remain substantially the same. This may allow the cassette assembly 9502 to be easily moldable.

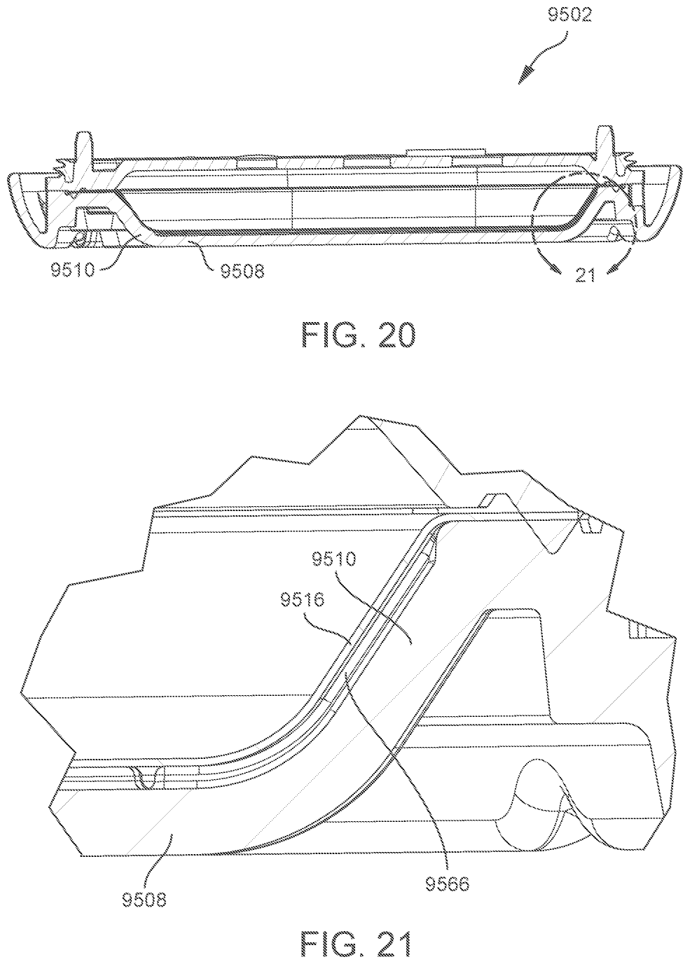

Referring now to FIGS. 19 and 20-23 when the reservoir 9536 is depleted from the full state shown in FIG. 19 (a cross section taken at the indicated cut plane in FIG. 12) to an empty state as shown in FIG. 20 (another cross section taken at the indicated cut plane in FIG. 12), the reservoir film 9516 may collapse onto the surface of the reservoir recess 9508. The vacuum created as fluid is pumped from the reservoir 9536 may not be sufficient to pull the reservoir film 9516 into the recessed ducts 9566. As best shown in FIG. 21 (a detailed view of the indicated region in FIG. 20), when the reservoir film 9516 is against the reservoir recess 9508 and recess wall 9510, the ducts 9566 may remain open to fluid flow. Thus, the ducts 9566 may form a network of interconnected fluid channels extending throughout the reservoir 9536 when the reservoir 9536 reaches an empty or near empty state. In the example embodiment, the ducts 9566 have a depth which is about 25% (e.g. 20-35%) of the thickness of the cassette base portion 9502 material which defines the reservoir recess 9508.

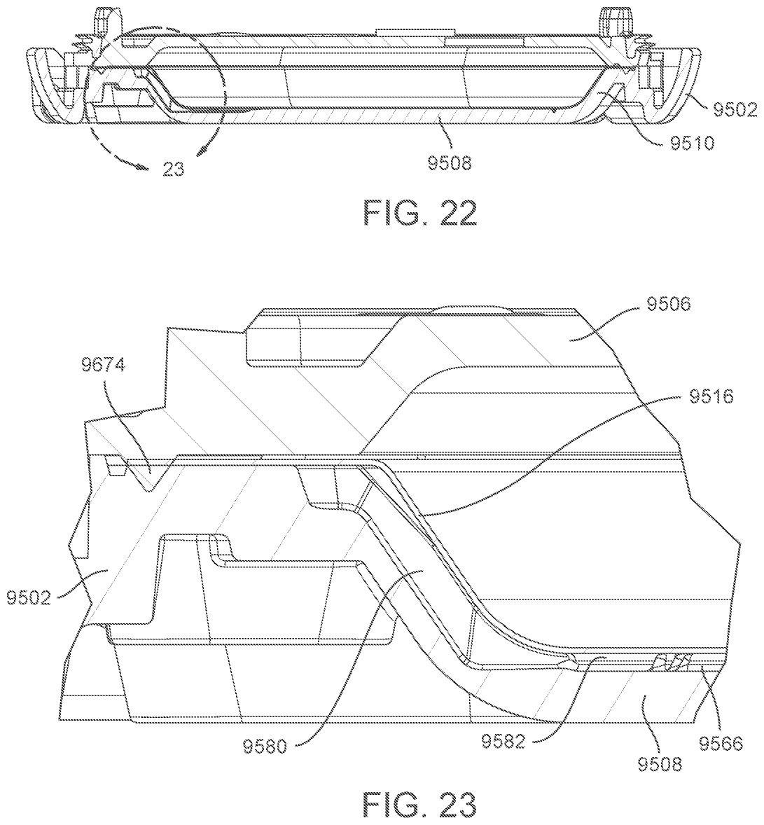

As shown in FIG. 22 (a cross section taken at the indicated cut plane in FIG. 12) and FIG. 23 (a detailed view of the indicated region of FIG. 22), the outlet duct 9580 and confluence region 9582 may not be obstructed by the reservoir film 9516 when the reservoir 9536 is empty or nearly empty. As a result, the reservoir outlet path 9544 (see, e.g. FIG. 10) may remain in fluid communication with large portions of the reservoir 9536 even when the reservoir film 9516 is pulled against the reservoir recess 9508 and recess wall 9510. This may help to prevent fluid from becoming isolated in pockets which are out of communication with the reservoir outlet path 9544 as the reservoir 9536 is collapsed. In the example embodiment, the outlet duct 9580 has a depth which is about ⅔ the thickness (e.g. 50-70%) of the of the cassette base portion 9502 material which defines the reservoir recess 9508.

By mitigating the chance for fluid to be isolated away from the reservoir outlet path 9544 as the reservoir 9536 is collapsed, the ducts 9566 may ensure that the reservoir 9536 can be more completely and consistently emptied. Since more of the fluid loaded into the reservoir 9536 may be utilized, the user, health systems, or insurers may experience a cost savings. Additionally, such a duct 9566 arrangement may increase the average therapy time a reservoir 9536 may support once filled. Another benefit may be an increase in the usefulness of a reservoir volume remaining determination made by a component of the delivery system 10. As the ducts 9566 may help to ensure that more of the fluid loaded into the reservoir 9536 can be used, the volume remaining determination may allow for more robust scheduling of therapeutic events executed by the drug delivery system 10 that are based on the volume remaining determination. This may be especially true when the reservoir 9536 is nearly empty.

As is also shown in FIG. 23 for example, the cassette top portion 9506 of the cassette assembly 9500 may be affixed to the cassette assembly 9500 by attaching it, at least at some part, onto the reservoir film 9516. As shown, the cassette top portion 9506 may include an energy director 9674. The energy director 9674 may serve to focus sonic welding energy at the apex of the energy director 9674 which abuts the reservoir film 9516. The energy director 9674 shown is substantially triangular in cross section and may be between 0.5 and 0.7 mm in height in certain examples. The apex may be formed by a right angle, a 600 angle, or any other suitable angle.

Referring now also to FIG. 24, a view of a cassette assembly 9500 with the cassette top portion 9506 exploded away and rotated to depict its underside, the energy director 9674 may be formed as a ring at the perimeter of the bottom surface of the cassette top portion 9506. The energy director 9674 may seat over the reservoir film 9516 positioned on the attachment surface 9540 of the cassette base portion 9502. A weld surface 9681 disposed radially outward of the valve membrane cover 9520 may also be included and may align with the energy director 9674 ring when the cassette top portion 9506 is in place. The reservoir film 9516 may be thin and compressible so as not to present a significant step in the surfaces to which the cassette top portion 9506 is to be welded. When the weld is formed, the cassette top portion 9506 and may couple to the weld surface 9681 and form a seal at the location of the weld between portions the cassette base portion 9502 inside of the weld surface 9681 and those outside. Thus, when a reusable housing assembly 106 is coupled to a cassette assembly 9500 there may be two redundant environmental seals which are generated (see also description of environmental sealing ring 9556 in relation to FIG. 10).

Coupling the cassette top portion 9506 to the rest of the cassette assembly 9500 in this manner may allow of the cassette assembly 9500 to have a smaller footprint. It may also be advantageous as it may allow for the reservoir 9536 to be made with a greater maximum volume without increasing the footprint of the cassette assembly 9500. This may be particularly desirable where the cassette assembly 9500 is designed for use as part of an ambulatory infusion device such as an insulin pumping device. The size of such devices may be seen as a significant detractor for users as the user either wears or carries (e.g. in a pocket or on a belt clip) the device throughout the day. Users may want to cover or hide such a device so as to not draw attention to the device or have the device get in the way of quotidian activities. A smaller device or a device which holds more drug volume without taking up more space may thus be seen as especially attractive.

In certain embodiments, one or more additional energy director may be included within the footprint of the energy director 9674 ring. As shown, a second energy director 9676 is disposed within the energy director 9674 ring. The cassette base portion 9502 may include a welding recess 9678 which may accept the second energy director 9676. During welding, the sonic energy may cause the energy directors 9674, 9676 to melt along with material in the cassette base portion 9502 and reservoir film 9516. The materials may flow together and firmly couple the parts together. The second energy director 9676 and weld recess 9678 may be positioned adjacent, but radially inward of the valve membrane cover 9520. This may ensure that pressure is evenly applied against the valve membrane cover 9520 to compressibly seal the valve membrane cover 9520 against the cassette base portion 9502.

As shown, a number of locating pins 9680 and receptacles 9682 may also be included. In the example, the locating pins 9680 extend from the cassette base portion 9502 and may be received in locating receptacles 9682 of the cassette top portion 9506. This may facilitate proper alignment of the cassette top portion 9506 prior to welding of the cassette top portion 9506 to the rest of the cassette assembly 9500.

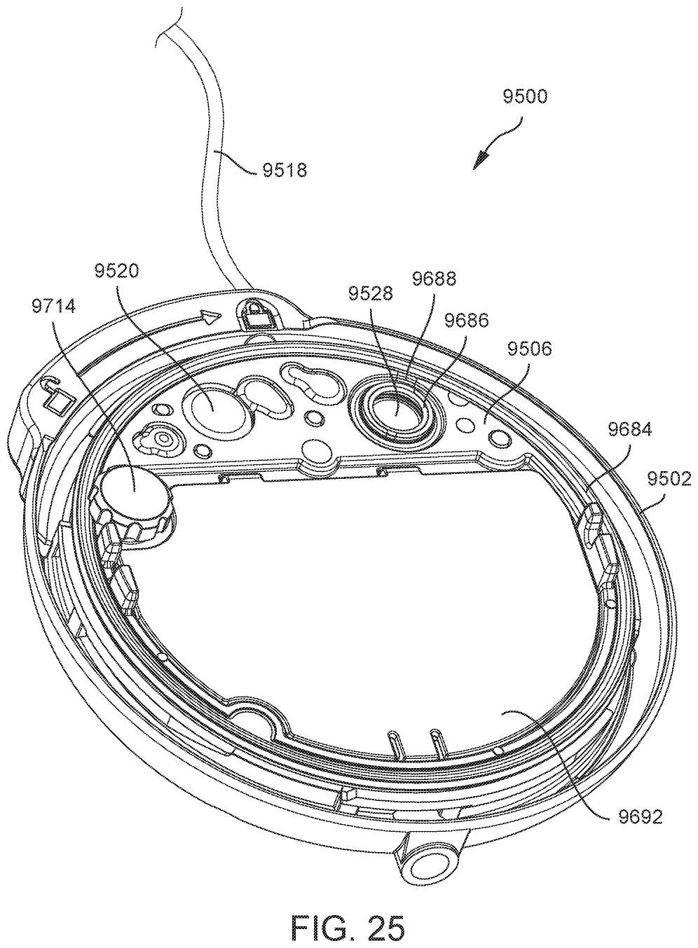

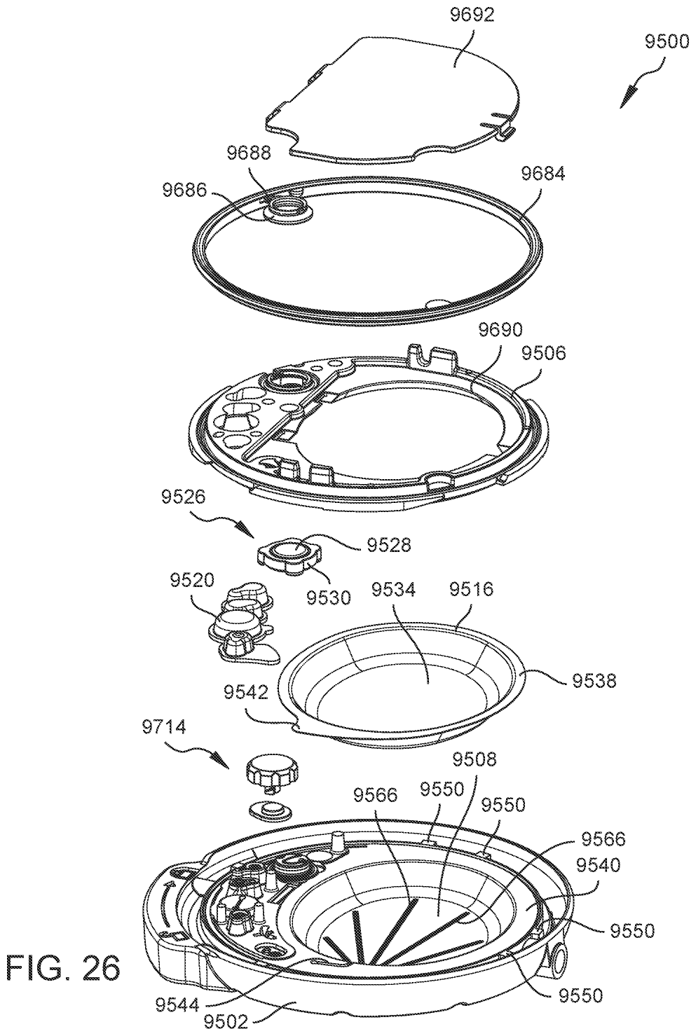

Referring now to FIGS. 25 and 26, another exemplary cassette assembly 9500 is depicted. The cassette assembly 9500 includes a cassette base portion 9502. The cassette base portion 9502 depicted includes an integrally formed reservoir recess 9508. A piece of reservoir film 9516 with a preformed region 9534 mimicking the shape of the reservoir recess 9508 may be coupled to the cassette base portion 9502 to define a reservoir 9536 which may be loaded with a drug such as any of those described herein. In the example embodiment, the reservoir recess 9508 includes a variety of recessed ducts 9566 (described elsewhere herein, see, e.g., FIGS. 17-18). The preformed region 9534 may not include corresponding features. When empty, the preformed region 9534 may seat within the reservoir recess 9508 such that the reservoir film 9516 abuts or is adjacent to the bottom surface of the reservoir recess 9508 and sits against the recess wall 9510, but does not extend into or occlude flow through the ducts 9566.

The reservoir film 9516 may include a peripheral rim region 9538. The peripheral rim region 9538 may be coupled to the cassette base portion 9502 at an attachment surface 9540 of the cassette base portion 9502 which may surround the reservoir recess 9508. An outcrop 9542 is also included in the shown peripheral rim region 9538. In the example embodiment, a reservoir outlet path 9544 is recessed into the attachment surface 9540. The outcrop 9542 of the reservoir film 9516 may seal over the reservoir outlet path 9544 to form an enclosed and leak tight fluid pathway out of the reservoir 9536.

A fill port 9524 may be included to provide fluid into the reservoir 9536. The fill port 9524 may extend directly to the interior volume of the reservoir 9536 through a side wall of the cassette base portion 9502. The fill port 9524 may be provided with a septum 9522. In some embodiments, the septum 9522 may be pierceable via a sharp 9586 (see, e.g., FIG. 47) on a filling implement 9584 (see, e.g. FIG. 47) and self-seal upon removal of the sharp 9586 so as to fluidically seal the reservoir 9536 once the reservoir 9536 has been loaded.

As described elsewhere herein, the fill port 9524, filling implement 9584, or a combination of the two may be configured to prevent reuse of the reservoir 9536 once a filling operation has been performed. Also as described elsewhere herein, the fill port 9524, filling implement 9584, or a combination of the two may also be configured to ensure access to the reservoir 9536 is restricted to only appropriate filling implements 9584. Other portions of a cassette assembly 9500 such as the cassette top portion 9506 and cassette base portion 9502 may include features for these purposes. The fill port 9524 in the example embodiment is disposed opposite the tubing 9518 extending from the cassette base portion 9502.

In the example embodiment, the cassette assembly 9500 is arranged to mate with a reusable housing assembly 106 via a twist lock type engagement (described elsewhere herein). The fill port 9524 in the example embodiment is disposed in a non-traversed region outside of the twist path of any of the coupling tabs of the reusable 106. The fill port 9524 may, for instance, be placed between a non-traversed region between two stop surfaces 9550. This may allow the fill port 9524 to be placed in a side wall of the cassette base portion 9502 while still allowing for a twist lock type engagement between a cassette assembly 9500 and reusable housing assembly 106.

The cassette assembly 9500 may further include a valve membrane cover 9520 including a number of regions which seat over a pumping chamber recess 9532 and valve features 9514 (such as those described above in FIGS. 2-8) included in the cassette base portion 9502. A volume sensor diaphragm assembly 9526 including a diaphragm 9528 and frame 9530 may also be coupled into the cassette assembly 9500 between the cassette base portion 9502 and the cassette top portion 9506. The volume sensor diaphragm assembly 9526 may be placed on a volume sensing station 9564 of the cassette base portion 9502. This may form a volume sensing chamber which may be monitored to determine volumes of fluid being transferred through the cassette assembly 9500. Further description is provided above in relation to FIG. 4.

The cassette top portion 9506 of the cassette assembly 9500 may include a compliant member 9684 which may be attached thereto. In the example, the compliant member 9684 may be disposed along a perimeter of the top surface of the cassette top portion 9506. The compliant member 9684 also includes a ring 9686 which surrounds the diaphragm 9528 when the cassette assembly 9500 is assembled. The example ring 9686 is integral with the compliant member 9684 and attached by a bridge 9688 which extends radially inward from the compliant member 9684. The compliant member 9684 and ring 9686 may serve as gaskets which form fluid tight seals when the reusable housing assembly 106 is attached to the cassette assembly 9500. The ring 9686 may form a sealed space connecting the volume sensing chamber with volume sensing hardware in the reusable housing assembly 106. The compliant member 9684 may generate an environmental seal which inhibits ingress of moisture, detritus, etc. into the dispensing assembly 100 when a reusable housing assembly 106 and cassette assembly 9500 are coupled to one another.

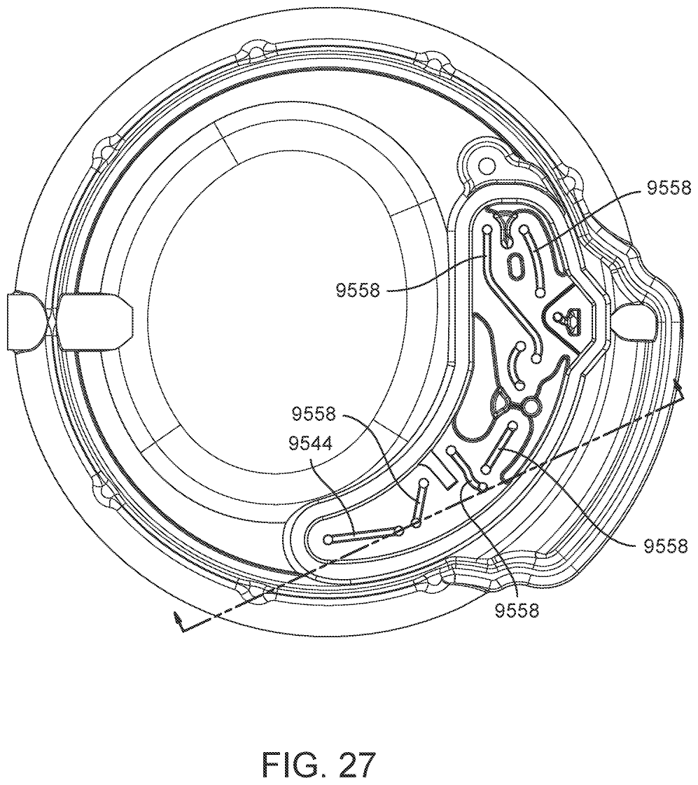

Referring now also to FIG. 27, the valve features 9514, pumping recess 9532, and volume sensing chamber may fluidically communicate with one another via flow channels 9558 (see, e.g., FIG. 27) defined in the opposing side of the cassette base portion 9502. A film sheet seal 9512 (further described in relation to FIG. 11) may be coupled onto the cassette base portion 9502 over the flow channels 9558 so as to form a seal over these flow channels 9558 which keeps fluid therein within the confines of the flow channels 9558.

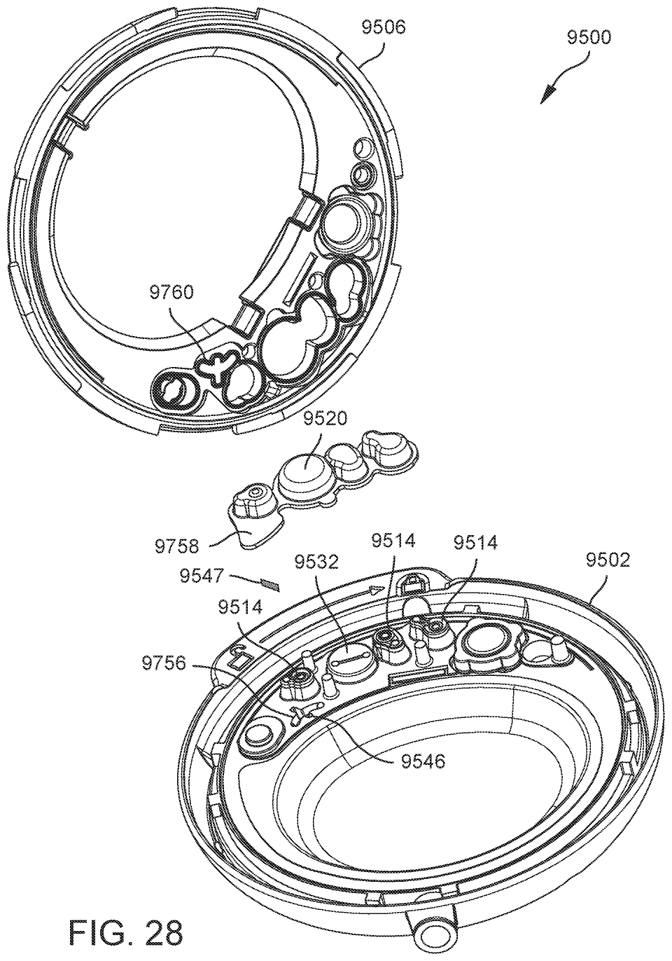

Referring now also to FIG. 28, flow channels 9756 may also be included on the same side of the cassette base portion 9502 as the valve features 9514, pumping recess 9532, and volume sensing chamber. In the example embodiment, a single flow channel 9756 is shown for purposes of example. The flow channel 9756 includes an air trap housing 9546. The air trap housing 9546 may retain an air trap 9547 which may be a screen or mesh insert as describe elsewhere herein. In the example embodiment, the valve membrane cover 9520 includes an outcrop region 9758 which may seal over the fluid channel 9756 when the cassette top portion 9506 is coupled in place in the cassette assembly 9500. As shown, the cassette top portion 9506 includes a sealing rib 9760 which mimics the shape of the fluid channel 9756 depression. When the cassette top portion 9506 is coupled into the cassette assembly 9500, the sealing rib 9760 may press against the outcrop region 9758. The pressure provided by the sealing rib 9760 may aid in creating a robust fluid tight seal around the flow channel 9756.

Referring again primarily to FIGS. 25-26, in some embodiments, the cassette assembly 9500 may not be filled by a patient 104 temporally proximate to its usage. Instead, a cassette assembly 9500 may be provided to a patient 104 in a prefilled state. For example, the patient 104 may receive cassette assemblies 9500 which have been filled by a manufacturer. Alternatively, a pharmacy may fill cassette assemblies 9500 which may be used to fill patient 104 prescriptions.

In embodiments where the cassette assembly 9500 is provided to a patient 104 in a prefilled state, the cassette assembly 9500 may be constructed to facilitate execution of one or more quality checks related to the prefilling of reservoir 9536. For example, the cassette assembly 9500 may include at least one fill verification feature. As shown, the cassette top portion 9506 of the cassette assembly 9500 may include a bay 9690 which may receive a cover 9692. The reservoir 9536 of the cassette assembly 9500 may be filled prior to installation of the cover 9692. Additionally, other portions of the cassette assembly 9500 may be constructed of a light colored (e.g. white opaque), translucent, transparent, or clear material. After filling, the cassette assembly 9500 may be inspected to verify that the filling operation was acceptable. Thus, the bay 9690 may be referred to herein as an inspection bay 9690. Such inspection may be performed manually, with a vision system, or both. Using a light or clear color for portions of the cassette assembly 9500 may facilitate various image processing operations in embodiments where a vision system is used. As shown, the cover 9692 is free from any holes or openings. When installed, the cover 9692 may prevent a patient 104 from exerting pressure against the reservoir 9536.

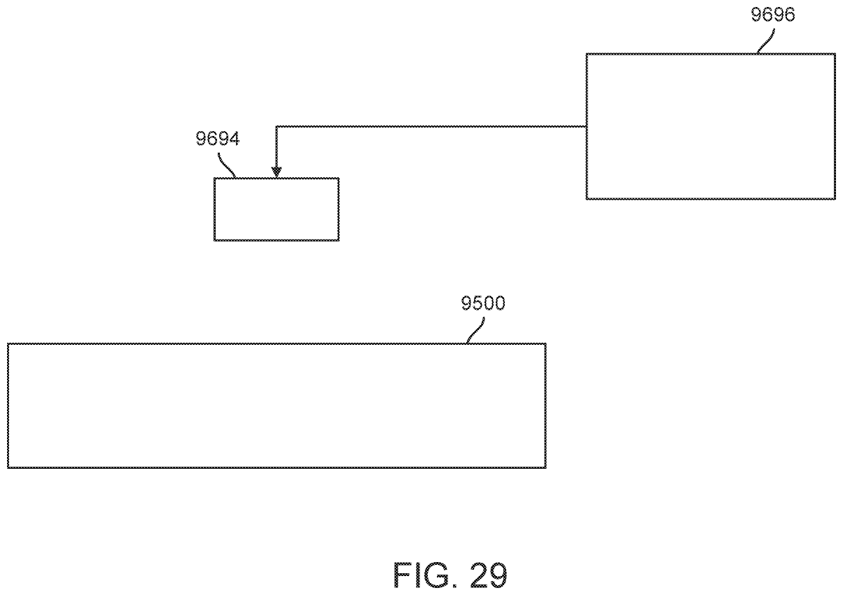

Referring now also to FIG. 29, in some embodiments, the cassette assembly 9500 may be inspected by at least one imager 9694 after it has been filled. The at least one imager 9694 may capture an image of the reservoir 9536. This image may be captured through the opening presented by the bay 9690 in the cassette top portion 9506 so as to allow for an unobstructed view of the reservoir 9536. An image processor 9696 may analyze the image to verify that the fill of the reservoir 9536 is in accordance with various defined criteria. For example, the analysis of the image by the image processor 9696 may determine whether the reservoir 9536 contains particulate or air (e.g. beyond some threshold amount). The analysis may also check for other undesirable scenarios. For example, the analysis may check for cloudiness, haze, or turbidity. The analysis may also include a range finder which verifies that the reservoir film 9516 is at a location which would be consistent with a desired fill volume. After it is determined that the reservoir 9536 conforms with defined criteria, the cover 9692 may be installed in place over the reservoir 9536.

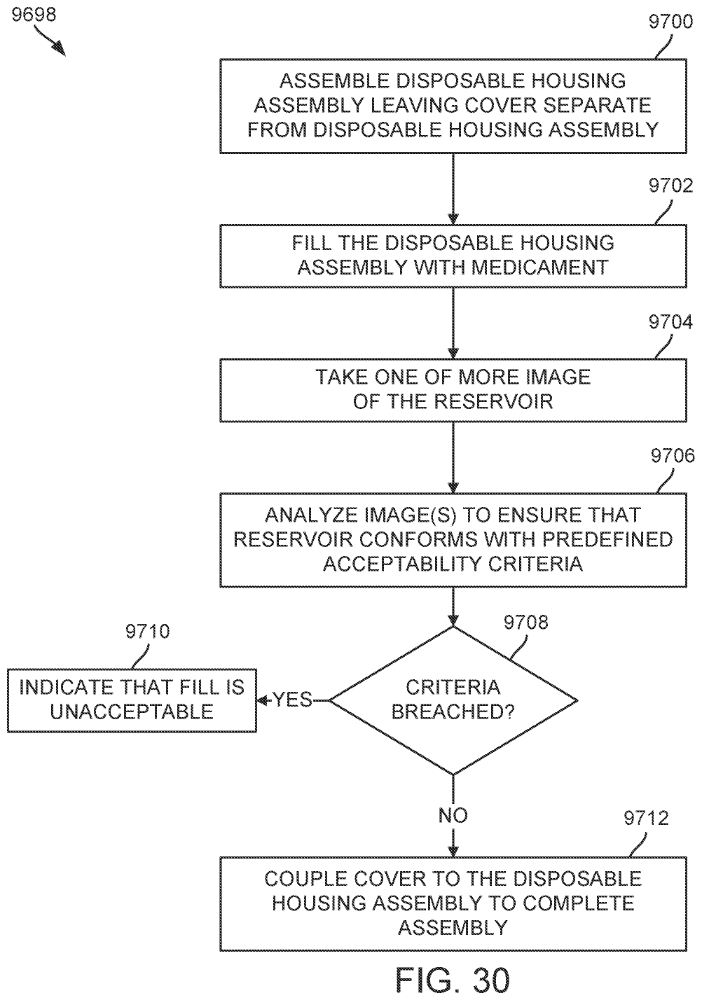

Referring now to FIG. 30, a flowchart 9698 depicting a number of example actions which may be executed to inspect a reservoir 9536 is shown. In block 9700, a cassette assembly 9500 may be assembled with the exception of the cover 9692. The reservoir 9536 of the cassette assembly 9500 may be filled in block 9702. One or more image may be taken of the reservoir 9536 in block 9704. The one or more image may be taken along a field of view which is unobstructed by components of the cassette assembly 9500. As mentioned above, an image or images of the reservoir 9536 may be captured through the window provided by the bay 9690 of the cassette top portion 9506. The image(s) may be analyzed in block 9706. If, in block 9708, acceptability criteria for the reservoir 9536 has been breached, a controller for the vision system may indicate that the cassette assembly 9500 is unacceptable in block 9710. If, in block 9708, the cassette assembly conforms with the acceptability criteria, the cover 9692 may be coupled into the bay 9690 in block 9712.

Referring now primarily to FIG. 31, a perspective break-away view of an example cassette assembly 9500 having a cover 9692 is shown. A segment of the cassette top portion 9506 and cover 9692 is broken away to illustrate retention features for holding the cover 9692 in place within the bay 9690 of the cassette top portion 9506. The cassette top portion 9506 includes a number of coupling members, in the example embodiment retention tabs 9716, which project from the wall of the bay 9690. The example cover 9692 includes a number of latch clips 9718. The example latch clips 9718 are formed as “L” shaped extensions which extend from the bottom surface of the cover 9692. A first edge 9720 of the cover 9692 includes two latch clips 9718. A latch clip 9718 is also included opposite the first edge 9720 and is flanked by two notches (only one shown in FIG. 31). The notches 9722 may cause the latch clip 9718 to be attached to the cover 9692 in a cantilevered fashion such that the latch clip 9718 may resiliently bend during installation of the cover 9692. In the example, the latch clips 9718 on the first edge 9720 may be displaced into position under their respective retention tabs 9716. The cover 9692 may then be rotated toward the cassette base portion 9502 until the bottom of the opposing latch clip 9718 contacts the top of its respective retention tab 9716. Further pressure may cause the latch clip 9718 to resiliently deflect around its respective retention tab 9716 and restore back to an unstressed state under the retention tab 9716. Thus, the cover 9692 may be assembled into the cassette assembly 9500 with little difficulty, however, may not be easily removed. As shown, the cover 9692 is a solid, monolithic piece of material with no openings or voids disposed interiorly to the sidewall of the cover 9692. Thus, the cover 9692 may protect the reservoir 9536 against tampering, needle sticks, and accidental application of pressure.

In other embodiments, the cover 9692 may be attached to the cassette assembly 9500 in any of a variety of manners. For example, the cover 9692 may be coupled into place via solvent bonding, adhesive, or welding (heat staking or sonic welding). Interference fits, snap fits, threaded engagements, bayonet mounts, etc. may also be used. In some embodiments, the cassette base portion 9502 or another portion of the cassette assembly 9500 may include slots or notches into which cooperating protuberances on the cover 9692 may be coupled to assembly the cover 9692 in place on the cassette assembly 9500. Preferably, once coupled into place, the cover 9692 may be difficult for a user to remove.

Referring now to FIG. 32 a perspective view of an alternative embodiment of a cassette assembly 9500 is depicted. The cover 9692 is again shown as a solid protective body which may be coupled in place over the reservoir 9536. In the example shown, the cover 9692 includes tabs 9770. The tabs 9770 of the cover 9692 may mate into receiving notches 9772 which are included in the cassette top portion 9506. In the example embodiment, the notches 9772 are positioned as alignment bodies 9774 which extend proud of the top face of the cassette top portion 9506. These alignment bodies 9774 may aid in coupling of the cassette assembly 9500 to a reusable housing assembly 106. The cover 9692 may be resiliently flexible so as to allow the cover 9692 to deflect as the tabs 9770 are snapped into the notches 9772.

Referring again primarily to FIGS. 25-26, the cassette assembly 9500 may include an occluder assembly 9714 in embodiments where the reservoir 9536 is prefilled prior to being received by a user. The occluder assembly 9714 may fluidically isolate a drug contacting surfaces in a first portion of the cassette assembly 9500 from drug contacting surfaces in a second portion of the cassette assembly 9500.

The first portion of the assembly 9500 may be constructed of materials which are compatible with any drug stored in the cassette assembly 9500 over long term storage timeframes. For example, the first portion may be constructed of materials compatible with the drug over several months to a year or more worth of storage. The first portion may include the reservoir 9536 and the reservoir outlet path 9544. The second portion may include of materials which are compatible with the drug for a shorter timeframe or tested to have an acceptable compatibility with the drug over a shorter duration. In some embodiments, the material of the second portion may only be in contact with the drug during a therapy and related activities (e.g. priming of the cassette assembly 9500). Depending on the embodiment, a reservoir 9536 may hold enough drug for a three day therapy whereafter the cassette assembly 9500 may be discarded and a new cassette assembly 9500 may take its place on a reusable housing assembly 106. The second portion may include all fluid contacting surfaces of the cassette assembly 9500 downstream of the occluder assembly 9714.

The occluder assembly 9714 may be constructed of materials having long term compatibility. Given the small area of the occluder assembly 9714 exposed to a drug or medicant, the occluder assembly, particularly the diaphragm 9724 may be formed from a halogenated butyl rubber, bromobutyl or a chlorobutyl. Alternatively, portions of the occluder assembly 9714 in contact with fluid in the first portion of the cassette assembly 9500 when the occluder assembly 9714 is in an occluding state may be constructed of materials having long term compatibility. Fluid contacting components in the first portion of the cassette assembly 9500 may be chosen to have long term compatibility with the drug. Fluid contacting components downstream of the occluder assembly 9714 may be selected to have at least short term compatibility with the drug. This may allow for the drug to be stored for long periods of time without ill effect while also allowing for a wider range of material choices in other components of the cassette assembly 9500 such as the valve membrane cover 9520. This may be particularly desirable for certain proteinaceous medicaments such as insulin.

The occluder assembly 9714 may establish multiple seals which isolate the fluid in the reservoir 9536 and reservoir outlet path 9544 from the rest of the cassette assembly 9500. Each seal may independent completely isolate the reservoir 9536 and reservoir outlet path 9544 from the rest of the cassette assembly 9500. At least two seals may be included, though greater numbers of seals are also possible. This redundancy may help to provide extra assurance that fluid may not leak through the occluder assembly 9714.

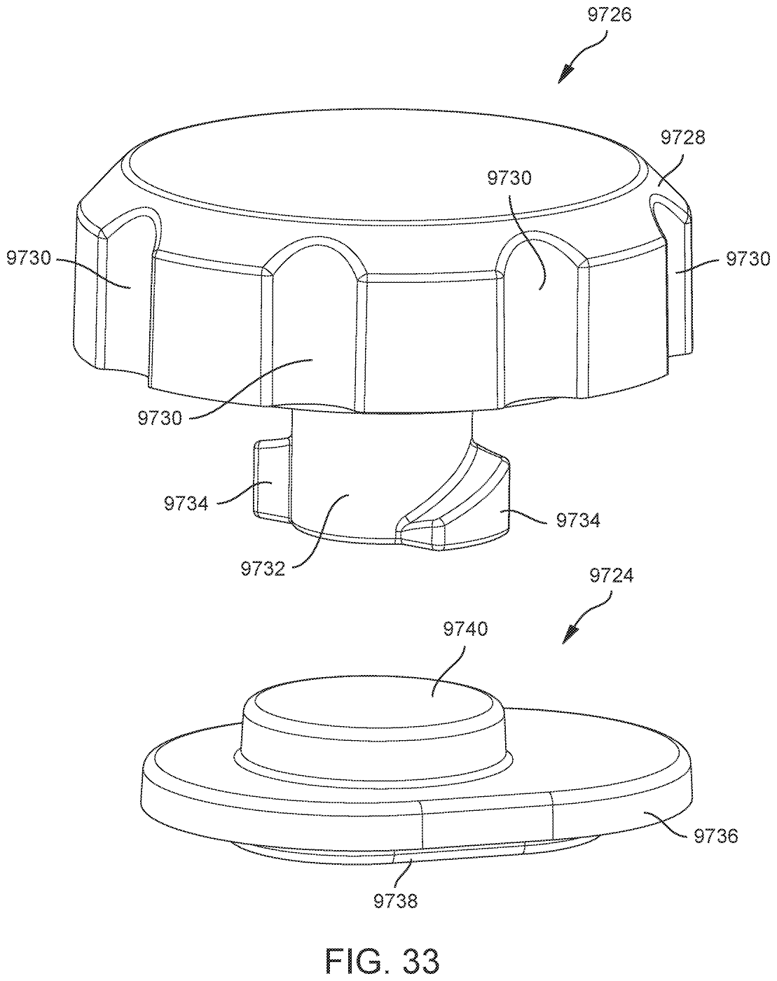

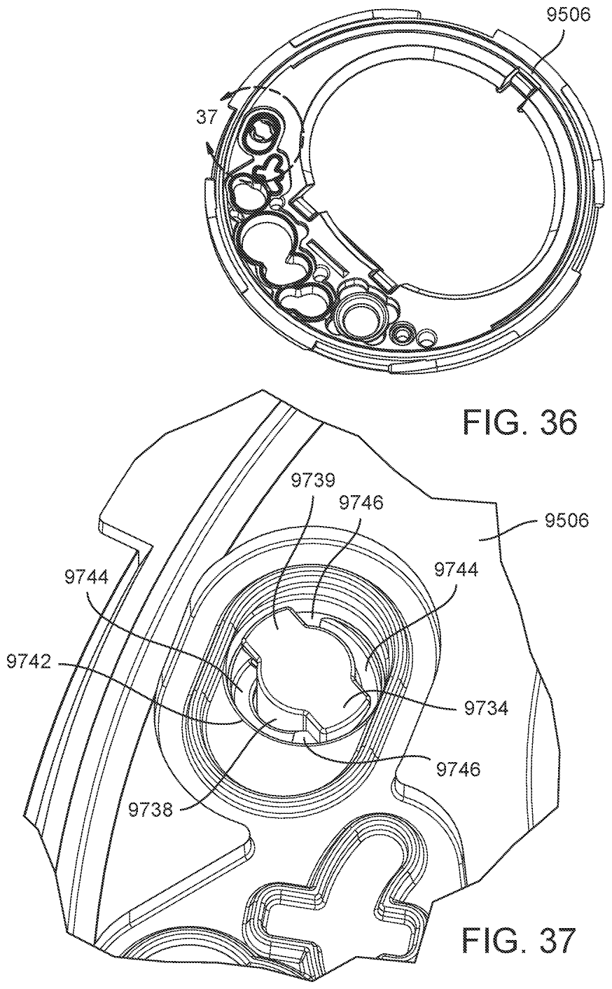

Referring now also to FIGS. 33-37, prior to initiation of the therapy, the occluder assembly 9714 or a portion thereof may be removed or brought into a non-occluding state. This may place first portion of the cassette assembly 9500 into fluid communication with the second portion of the cassette assembly 9500. In some embodiments, the reusable housing assembly 106 may be prevented from coupling with the cassette assembly 9500 if the occluder assembly 9714 is in an occluding state. Thus, the occluding state may also serve as a mating prevention state. For example, a portion of the occluder assembly 9714 may stand proud of the rest of the cassette assembly 9500. When a user attempts to attach a reusable housing assembly 106 to the cassette assembly 9500, the reusable housing assembly 106 may contact the proud portion of the occluder assembly 9714 and be inhibited from displacing further toward the cassette assembly 9500. Coupling features of the cassette assembly 9500 and reusable housing assembly 106 may be out of engagement with one another when the reusable housing assembly 106 is bottomed out on the proud portion of the occluder assembly 9714.

In the example embodiment, and as best shown in FIG. 33, the occluder assembly 9714 may include a diaphragm 9724 and an actuator 9726. The actuator 9726 may include a knob 9728. The knob 9728 may include a number of flutes 9730 which may facilitate gripping by a patient 104 or removal tool. A rod 9732 may extend from the knob 9728 and may include two ramp features 9734 which may be located at a terminal end thereof. The ramp features 9734 are disposed radially opposite one another. In alternative embodiments, the number of ramp features 9734 may be greater and may be spaced about the rod 9732 at some predefined pattern. In some embodiments, the ramp features 9734 may be placed at even angular increments from one another or may be disposed about the rod 9732 at irregular angular increments.

The diaphragm 9724 includes a main body 9736. Plateau regions 9738, 9740 may be included on the top and bottom side of the main body 9736. The plateau region 9740 on the top of the main body 9736 may align with the rod 9732 when assembled into the cassette assembly 9500. The plateau region 9738 included on the bottom of the main body 9736 may provide a contact face for sealing against one or more valve gating flow between the first and second portion of the cassette assembly 9500. The diaphragm 9724 (or at least the plateau region 9738) may be made of a compressible material which possesses long term compatibility with the drug contained within the cassette assembly 9500. In some embodiments, the diaphragm 9724 and the septum 9522 may be constructed of the same material. In some embodiments, the material of the diaphragm 9724 may be chlorobutyl elastomer.

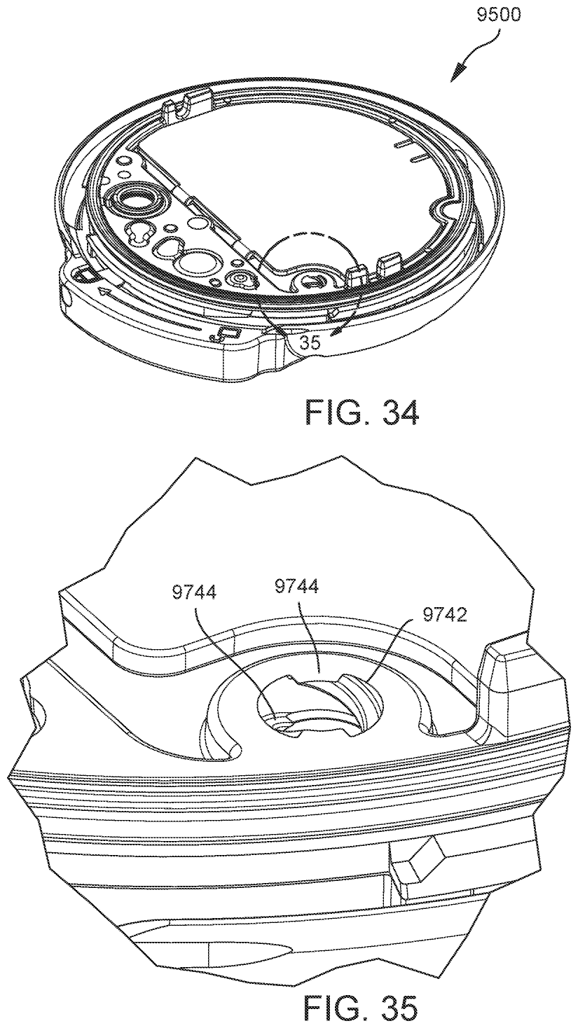

Referring now primarily to FIG. 35 (a detailed view of the indicated region of FIG. 34), the cassette assembly 9500 may include a threaded port 9742. The threaded port 9742 may be outfitted with a set of threads 9744 which may have a pitch selected to cooperate with the ramp features 9734 on the actuator 9726. The threaded port 9742 may be positioned over a section in the cassette base portion 9502 including the above mentioned one or more valve gating flow between the portion of the cassette assembly 9500.

Primarily referring to FIG. 37 (a detailed view of the indicated region of the underside of the cassette top portion 9506 shown in FIG. 34), the actuator 9726 may be threaded into the threaded port 9742. As shown, each of the threads 9744 in the threaded port 9742 may guide the ramp portions 9734 of the actuator 9726 onto respective retention regions 9746. In the example shown, the retention regions 9746 are shelf like expanses which are continuous with the threads 9742. The retention regions 9746 may include detents in certain embodiments and may serve to hold the actuator 9726 in place when fully threaded into the threaded port 9742. When the ramped portions 9734 are parked on the retention regions 9746, the actuator 9726 may be in an occluding position or state. In this position, the actuator 9726 may press into the diaphragm 9724 compressing or deflecting the diaphragm 9724 against a valve seat located opposite the rod 9732 of the actuator 9726. Flow through the valve seat may be blocked when diaphragm is pressed against the valve seat.

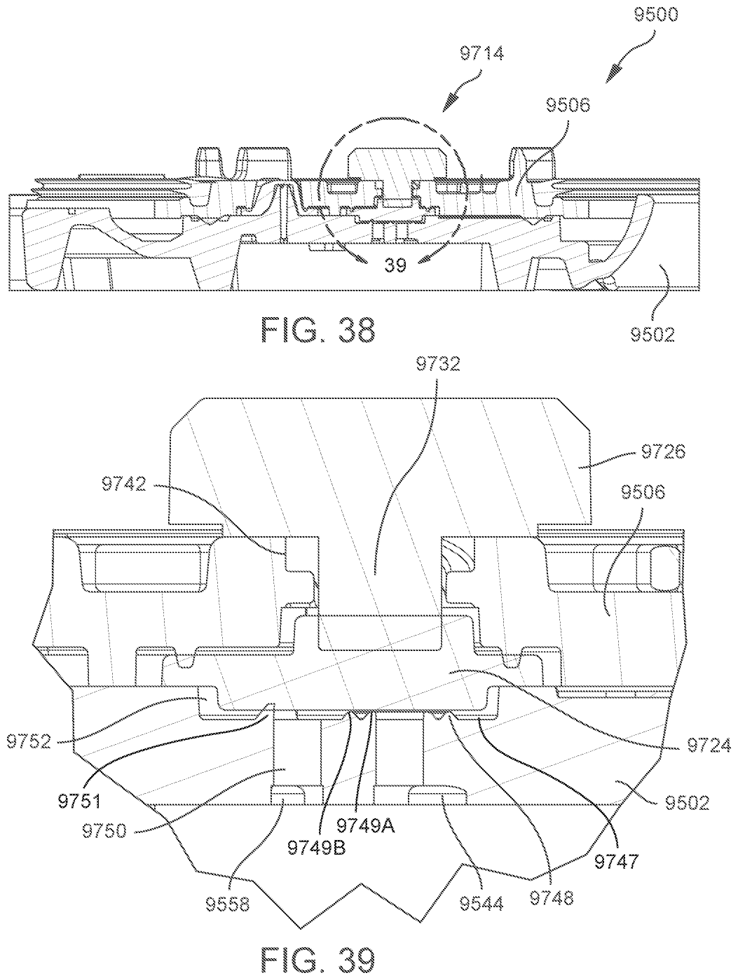

Referring now primarily to FIG. 38 (a cross section taken at the indicated plane in FIG. 27) and FIG. 39 (a detailed view of the indicated region of FIG. 38), a cross sectional view of an example occluder assembly 9714 is shown. The actuator 9726 is shown in the occluding position. The diaphragm 9724 is pressed against a reservoir outlet valve 9748 which gates flow from the reservoir outlet path 9544 (see, e.g., FIG. 26) to the rest of the cassette assembly 9500. For ease of illustration, a portion of the actuator 9726 extends into the diaphragm 9724. In practice, the actuator 9726 would instead compress the diaphragm 9724. Thus, the actuator 9726 may firmly close the reservoir outlet valve 9748 when installed.

The reservoir outlet valve 9748 is shown as a volcano type valve in the example embodiment. Specifically, the reservoir outlet valve 9748 is depicted as a double volcano type valve. When the actuator 9726 is in the occluding position, the diaphragm 9724 may be pressed against the reservoir outlet valve 9748 such that the diaphragm is compressed upon each valve seat 9749A, B of the double volcano valve. As a result, the occluder assembly 9714 may isolate the first portion of the cassette assembly 9500 from the rest of the cassette assembly 9500 at a multiplicity of redundant seals.

Also as shown, the diaphragm 9724 may be compressed in place between the cassette top portion 9506 and cassette base portion 9502 within the cassette assembly 9500. This compression may create seals between the diaphragm 9724, the cassette top portion, and the cassette base portion 9502. The diaphragm 9724 may, for example, be sealed around the periphery of a depression 9747 in which the reservoir outlet valve 9748 is disposed. In some embodiments, redundant seals similar to those created against the outlet valve seats 9749A, B may be present around the periphery of the depression. This may form a sealed occluder volume 9752 through which fluid may pass during therapy. Preferably the occluder volume 9752 may be sized to be minimal so as to limit the amount of dead space within the flow path through the cassette assembly 9500.

When the actuator 9726 is threaded out of the threaded port 9742, the diaphragm 9724 may restore from its compressed or deflected state. In its resting state, the diaphragm 9724 may displace away from the reservoir outlet valve 9748 bringing the reservoir outlet valve 9748 into an open state. In the open state, fluid may flow through the occluder volume 9752 to an occluder outlet 9750 which is plumbed into the flow channels 9558 included on the backside of the cassette assembly 9500. Thus, when the actuator 9726 is extracted from the threaded port 9742, the first portion of the cassette assembly 9500 may be placed into fluid communication with the second portion of the cassette assembly 9500. The occluder assembly 9714 may transition to a flow permitting state when the actuator 9726 has been unthreaded from the position shown in FIG. 39 to at least a second position. As mentioned above, depending on the embodiment, it may be necessary to completely remove the actuator 9726 in order to couple the cassette assembly 9500 to a reusable housing assembly 106 before initiating therapy.

As shown, one or more wall 9751 may be provided around a portion of the occluder outlet 9750. The wall(s) 9751 may be more proud of the depression 9747 than the valve seats 9749A, B of the reservoir outlet valve 9748. The wall(s) 9751 may act as a restoring projection which helps to ensure that once the occluder assembly 9714 is brought to a non-occluding state, the diaphragm 9724 is pushed away from the reservoir outlet valve 9748 aiding in opening of the reservoir outlet valve 9748. Additionally, the partial wall(s) 9751 around the occluder outlet 9750 may ensure that the occluder outlet 9750 is unlikely to ever be blocked off by the diaphragm 9724. Thus, the partial wall(s) 9751 may ensure that flow through the occluder assembly 9714 is unimpeded once the occluder assembly 9714 is brought into a non-occluding state.

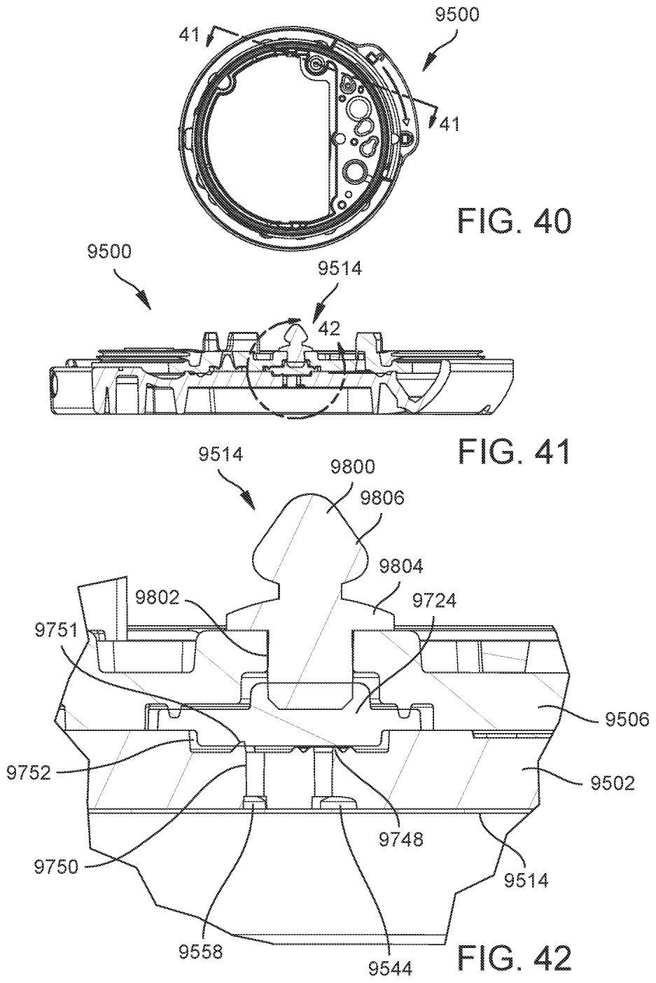

Referring now to FIG. 40-42, views of an embodiment of a cassette assembly 9500 with another occluder assembly 9714 are depicted. As shown, the occluder assembly 9714 is substantially the same as that shown in FIGS. 38-39, however, a bung 9800 type actuator for the occluder assembly 9714 is included in place of the actuator 9726. The bung 9800 may be constructed of a compressible elastomer material. The bung 9800 may be pressed into a tapered port 9802 and may include a shoulder 9804 which prevents displacement of the bung 9800 into the tapered port 9802 beyond a certain point. The diameter of the bung 9800 may be larger than at least the smallest section of the tapered port 9802. Thus, an interference fit may be created when the bung 9800 is installed which aids in retaining the bung 9800 in position within the tapered port 9802.

When pressed into the tapered port 9802, the bung 9800 may displace and compress the diaphragm 9724 against the reservoir outlet valve 9748 closing off flow therethrough. The end of the bung 9800 which contacts the diaphragm 9724 may also deform and bulge outward when advanced into the diaphragm 9724. This bulging may further aid in retaining the bung 9800 in place within the tapered port 9802 during storage. The bung 9800 may include a nub 9806 on an exposed portion of the bung 9800 which may be grasped in order to remove the bung 9800. The bung 9800 may be manually removed or pulled out of the tapered port 9802 with a removal tool. As in FIG. 39, at least one wall 9751 may be provided around a portion of the occluder outlet 9750. The partial wall 9751 may help to drive the diaphragm 9724 away from the reservoir outlet valve 9748 when the bung 9800 is removed.

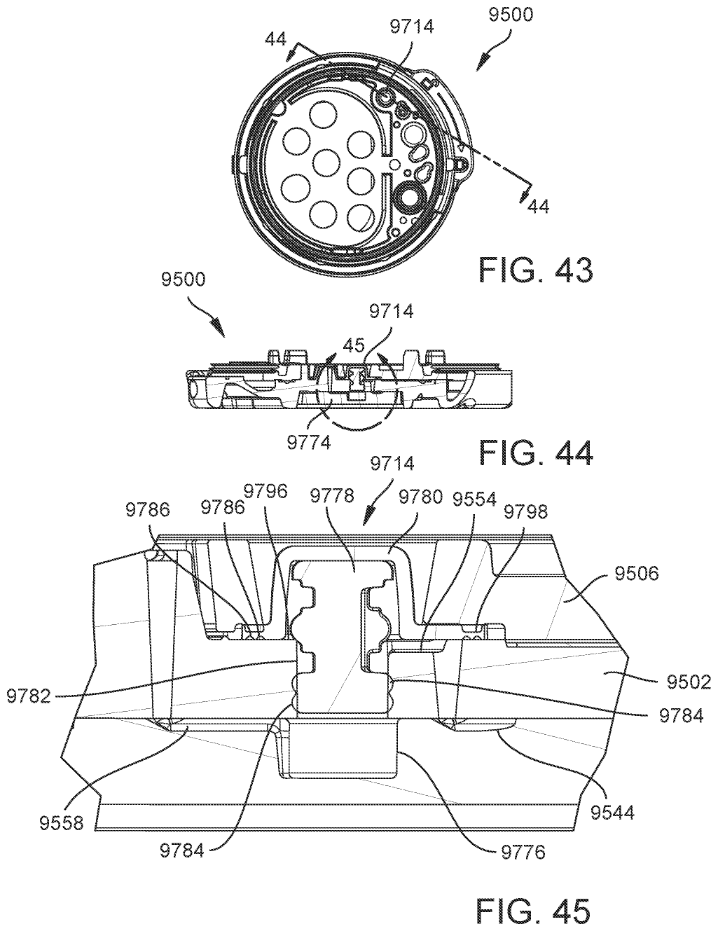

Referring now to FIGS. 43-45, views of an embodiment of a cassette assembly 9500 with another occluder assembly 9714 are depicted. Referring primarily to FIG. 45, the occluder assembly 9714 may include a shuttle body 9778 which acts as the actuator for the occluder assembly 9714. The shuttle body 9778 may be displaceable along an occluder channel 9782 which is in fluid communication with the reservoir outlet path 9544 and a flow channel 9558 leading to the second portion of the cassette assembly 9500. In the example, the occluder assembly 9714 includes a diaphragm 9780 which is captured between the cassette base portion 9502 and cassette top portion 9506 of the cassette assembly 9500. A sealing plate 9774 (described in further detail above in relation to FIG. 11) which may seal and/or partially define flow channels 9558 of the cassette assembly 9550 may also define a portion of the occluder assembly 9714. As shown, the sealing plate 9774 may include a well 9776. The channel 9780, in the example embodiment, is defined by the well 9776, the diaphragm 9780, and a passage extending through the cassette base portion 9502.

The cassette base portion 9502 may include at least one rib 9786 which may surround the occluder channel 9782. In the example embodiment a set of ribs 9786 is included. The cassette top portion 9506 may include compression projections 9798 which may align over the ribs 9786 when the cassette top portion 9506 is coupled to the cassette base portion 9502. These ribs 9786 may form a layer of redundant seals between the diaphragm 9780 and the cassette base portion 9502 when the diaphragm 9780 is captured and compressed between the cassette base portion 9502 and the cassette top portion 9506. Such an arrangement may also be included in the other occluder assembly 9714 embodiments (e.g. that shown in FIG. 39 or FIG. 42).

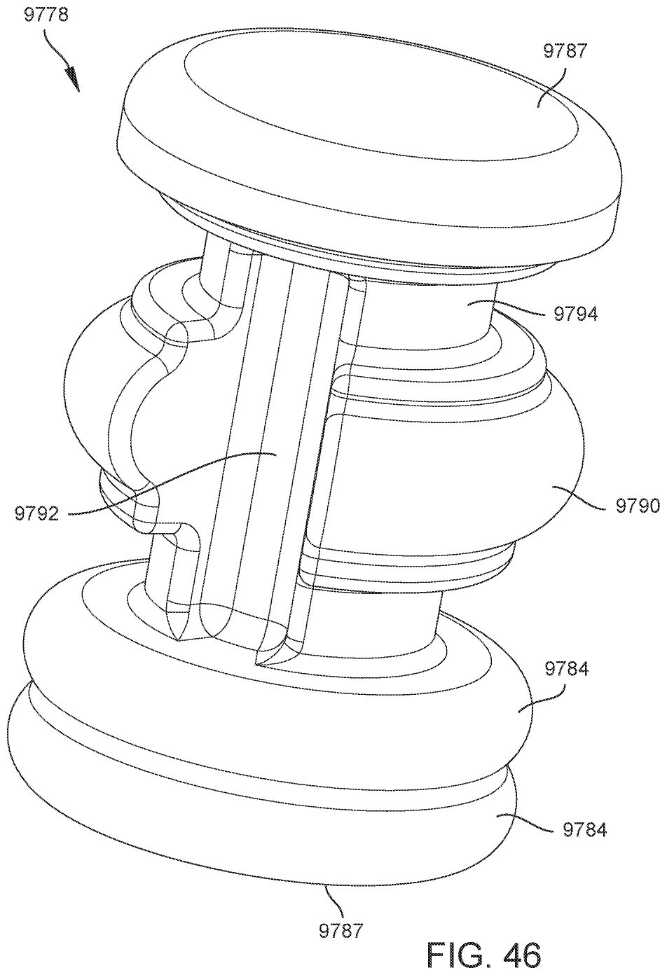

Referring now also to FIG. 46, a perspective view of the shuttle body 9778 is shown. The shuttle body 9778 may include at least one sealing interface 9784. In the example embodiment, two sealing interfaces 9784 which are shown as protruding ridges on the shuttle body 9778 are included. The sealing interfaces 9784 are included in one of the enlarged end sections 9788 of the shuttle body 9778. The sealing interfaces 9784 may fluidically seal against the walls of the occluder channel 9782 and may also generate stiction which resists unintentional displacement of the shuttle body 9778. In the example embodiment, the entire shuttle body 9778 is a monolithic part which may, for example be constructed of molded chlorobutyl elastomer (the diaphragm 9780 may also be made of this material). In other embodiments, the sealing interfaces 9784 may be o-rings which are assembled onto the shuttle body 9778 during manufacture of a disposably housing assembly 9500.

Referring primarily to FIGS. 45-46, a cassette assembly 9500 may be packaged with the shuttle body 9778 in an occluding position (shown in FIG. 45). In the occluding position, the shuttle body 9778 may be located such that the sealing interface(s) 9784 of the shuttle body 9778 prevent flow from the reservoir outlet path 9544 to the flow channels 9558 in the second portion of the cassette assembly 9500. In the example embodiment, the sealing interface(s) 9784 are sealed against the wall of the occluder channel 9582 intermediate the entry points of the reservoir outlet path 9544 and fluid channel 9558 into the occluder channel 9582. As shown, the two sealing interfaces 9784 in the example embodiment provide redundant seals which isolate fluid prefilled into the reservoir 9536 from the second portion of the cassette assembly 9500.

When the user is preparing the cassette assembly 9500 for use, the user may press on the diaphragm 9780 to displace the shuttle body 9778 into a flow permitting state. Pressing may be accomplished manually (e.g. via a thumb or other digit) or via a tool such as a priming and/or filling aid. Examples of such aids are described in U.S. Pat. No. 10,080,704, Issued Sep. 25, 2018, entitled Apparatus, System and Method for Fluid Delivery, to Lanigan et al., Attorney Docket No. M62 which is hereby incorporated by reference herein in its entirety. Alternatively, the reusable housing assembly 106 may include an actuator which may be powered to displace the shuttle body 9778. In some embodiments, a reusable housing assembly 106 may include a feature (e.g. a ramped projection) which depresses (e.g. as it is swept passed) the shuttle body 9778 as the reusable housing assembly 106 is coupled to the cassette assembly 9500. When in a flow permitting state, the shuttle body 9778 may be disposed such that the sealing interface(s) 9784 are within the well 9776 so as to place the reservoir outlet path 9544 into communication with the fluid channel 9558 leading from the well 9776.

As shown best in FIG. 46, the shuttle body 9778 includes an enlarged mid region 9790 in addition to the enlarged end regions 9587. The enlarged mid region 9790 may include a recess 9792 which extends into the stem portion 9794 of the shuttle body 9778. The recess 9792 may provide a flow pathway for fluid to flow through the enlarged mid region 9790. The enlarged mid region 9790 may be dimensioned so as to catch against a step 9796 (see, e.g. FIG. 45) included in the occluder channel 9782. Thus, the enlarged mid region 9790 may help to hold the shuttle body 9778 in an occluding state during storage. As the shuttle body 9778 may be made of a compressible material, sufficient pressure on the diaphragm 9780 may cause the enlarged mid region 9790 to deform allowing it to clear the step 9796 during actuation to a flow permitting state. The enlarged mid region 9790 may also fill space within the occluder channel 9782 which may help to limit dead space in the occluder assembly 9714.

Though not shown in FIG. 45, in some embodiments, the diaphragm 9780 and the shuttle body 9778 may be integral with one another. When the shuttle body 9778 is moved to a flow permitting state, the diaphragm 9780 may be advanced in the direction of the well 9776. Thus, the amount of open volume above the shuttle body 9778 may be reduced when the shuttle body 9778 is displaced. This may help to maximize usage of the fluid preloaded into the reservoir 9536 as dead volume in the occluder assembly 9514 may be minimized.

Though the example embodiment is depicted with a sealing plate 9774, it is also possible to include a similar occluder assembly 9714 in cassette assemblies 9500 that use a film sheet seal 9512 instead of a sealing plate 9774. In such embodiments, the film sheet seal 9512 would close a bottom end of the occluder channel 9782. The thickness of the cassette base portion 9502 may be increased in the area of the occluder channel 9782 such that the length of the occluder channel 9782 may remain substantially the same. Additionally, the width of the occluder channel 9782 adjacent the film sheet seal 9512 may be increased to a size which provides clearance around the sealing interface(s) 9784 of the shuttle body 9778. This may allow for fluidic communication to be established between the reservoir outlet flow path 9544 and second portion of the cassette assembly 9500 when the shuttle body 9778 is actuated to a flow permitting state.

Referring now to FIG. 47, as mentioned above, the fill port 9524 may include one or more features which may inhibit reuse of a cassette assembly 9500 or restrict access to the fill port 9524 by inappropriate filling implements 9584. This may be accomplished through the use of various keying features incorporated into the fill port 9524, filling implement 9584, and/or other components of the cassette assembly 9500. A filling implement 9584 may include a portion which mates with the filling port 9524, a feature of the cassette base portion 9502, top cover 9506, or a combination thereof when filling of the reservoir 9536 is performed. The interfacing portion 9588 of the filling implement 9584 may, for example, engage into or mate around the filling port 9524. The sharp 9586 of the filling implement 9584 may not be able to establish fluid communication (e.g. may be too short) with the interior volume of the reservoir 9536 unless the interfacing portion 9588 of the filling implement 9584 is properly mated with the filling port 9524 and/or other portion of the cassette assembly 9500. In the event that the interfacing portion 9588 of the filling implement 9584 is not configured to mate with the filling port 9524 and/or other portion of the cassette 9500, the user may be prevented from filling the reservoir 9536.

By restricting the filling implements 9584 which may be used with a cassette assembly 9500 a number of potential advantages may be possible. For example, the user may be prevented from using a filling implement 9584 having a sharp 9586 which is excessively long. This may mitigate any opportunity that the tip of the sharp 9586 may come into contact with the reservoir film 9516 and cause damage. Additionally, it may ensure that filling implements 9584 contain an appropriate volume of fluid for the particular cassette assembly 9500 about to be loaded with fluid. For example, a user may be prevented from using a syringe capable of holding more than the maximum volume of the reservoir 9536 and overfilling the cassette assembly 9500. The filling implement 9584 may also be designed so as to be specific to specific drug types or specific concentrations of certain drugs. Using insulin as an example, the drug may be available for injection in U 100 and U 200 concentrations. Cassette assemblies 9500 and filling implements 9584 may be constructed to be specific to each concentration and loading of the improper concentration into a cassette assembly 9500 may be prevented.

In the example shown in FIG. 47, the filling implement 9584 is depicted as a syringe. It should be appreciated that any of a wide variety of filling implements 9584 may alternatively be used. For example, pen type delivery devices, pumps, syringes, gravity feed systems, pressurized ampoules or vials, etc. may be used. In some examples, an intervening adapter between the filling implement 9584 and the cassette assembly 9500 may also be used. As shown, the filling implement 9584 includes a round interfacing portion 9588 which is not intended for use with the cassette assembly 9500 depicted in FIG. 47. The fill port 9524 of the cassette assembly 9500 includes a polygonal (rectangular in the example, though any suitable shape is possible) bore 9590. At least one dimension of the polygonal bore 9590 may be sized so as to prevent the interfacing portion 9588 of the filling implement 9584 from properly engaging with the filling port 9524. Thus, an improper filling implement 9584 may be inhibited from gaining access to the reservoir 9536. A filling implement 9584 having a rectangular interfacing portion 9588 may, however, fit within the polygonal bore 9590 and be able to access the interior volume of the reservoir 9536.

In other embodiments, the interfacing portion 9588 may also be polygonal in cross section, but dimensioned so as to only fit within a particular type of cassette assembly 9500. For example, the interfacing portion 9588 may have a triangular or star shaped cross section which is incompatible with the polygonal bore 9590 shown. The polygonal interfacing portion 9588 may, however, cooperatively engage with the fill port 9584 of a different cassette assembly type 9500 which may perhaps be intended for use with a different drug concentration. In some embodiments, the bore in the fill port 9524 may be round while still having at least one dimension (e.g. diameter, minor axis, or other axis of symmetry etc.) that prevents interfacing with inappropriate filling implements 9584. In general, the bore of a fill port 9524 may be a negative version of the interfacing portion 9588 of the appropriate filling implement 9584 so as to generate a lock and key type arrangement. Cooperating round, polygonal, oblong, or any other cooperating geometries may be used for a given fill port 9524 and filling implement 9584 interfacing portion 9588 pair.

Where different types of cassette assemblies 9500 are used, the cassette assemblies 9500 may include other differentiating features. These may help a user quickly determine the type of cassette assembly 9500 they are looking at. In some examples a first type of cassette assembly 9500 may be clear, translucent, or light in color (e.g. white) while a second type of cassette assembly 9500 may be opaque, or dark in color (e.g. black).

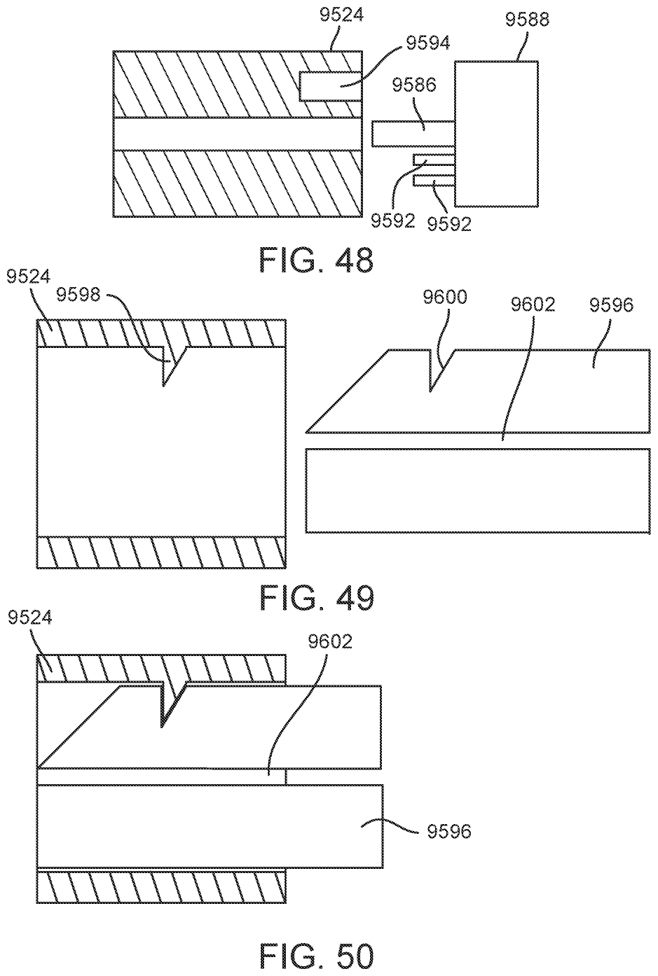

Referring now to FIG. 48, in another embodiment access to the interior volume of the reservoir 9536 may be restricted by a cooperating arrangement of projections 9592 and receptacles 9594 included on the fill port 9536, filling implement 9584, and/or another portion of the cassette assembly 9500 (such as the cassette base portion 9502 or cassette top portion 9506). The filling implement 9584 may include at least one projection 9592, receptacle 9594, or some combination of both. The cassette assembly 9500 may include cooperating projections 9592 and/or receptacles 9594 which may mate with those on the filling implement 9584 in the event that the proper filling implement 9584 is used. Improper filling implements 9584 may not include an arrangement of projections 9592 and/or receptacles 9594 which is capable of properly interfacing with those on the cassette assembly 9500. Projections 9592 may extend in a direction which is substantially parallel to the axis of the sharp 9586. Any cross-sectional shape projection 9592 may be used. In some embodiments, the cross-sectional shape of a projection 9592 may change or taper as the distance from its attachment point to the filling implement 9584 increases. For example, rounded nubs may be used in certain examples. The receptacles 9594 may be formed as a negative of their respective projections 9592.

As shown in FIG. 48, a filling port 9524 is depicted and includes a receptacle 9594. Though a fill port 9524 is shown, the receptacle 9594 may be included on any suitable part of the cassette assembly 9500. In other embodiments, receptacles 9594 may be included on the cassette base portion 9502 or cassette top portion 9506. The fill port 9524 shown in FIG. 48 is intended to engage with a filling implement 9584 having a single projection 9592 which is sized to fit within the receptacle 9594. The interfacing portion 9588 of an inappropriate filling implement 9584 is shown in FIG. 48. The example interfacing portion 9588 includes two projections 9592. If a user were to attempt to engage the example interfacing portion 9588 with the fill port 9524 shown in FIG. 48, the projections 9592 would not fit within the receptacle 9594 of the fill port 9524. As a result, the user would be unable to access the reservoir 9536 within the cassette assembly 9500.

In some embodiments, it may be desirable that the cassette assembly 9500 include at least one projection 9592. By including a projection 9592 on the cassette assembly 9500, inappropriate filling implements 9584 may be held away from the cassette assembly 9500 in the event a user attempts to introduce the wrong filling implement 9584. As a result, the sharp 9586 of an inappropriate filling implement 9584 may not be able to extend into communication with the interior volume of the reservoir 9536.

Referring now to FIG. 49 and FIG. 50, in certain embodiments, the filling implement 9584 may include a detachable member or assembly 9596. A detachable member 9596 may be held in place on the filling implement 9584 by a frangible type connection, friction fit, or any other suitable connection. When the interfacing portion 9588 of the filling implement 9584 is mated with a cassette assembly 9500, the detachable member 9596 may be disassociated with the filling implement 9584 and be left behind when the filling implement 9584 is removed. The detachable member 9596 may be retained by the cassette assembly 9500 such that its removal is difficult. While retained by a cassette assembly 9500 the detachable member 9596 may present a mechanical interference to a subsequent filling implement 9584 being attached. As the space in the cassette assembly 9500 for a detachable member 9584 included on the subsequent filling implement 9584 has already been claimed, the subsequent filling implement 9584 may not be able to mate with the cassette assembly 9500. Thus, the detachable member 9596 may prevent reuse of a cassette assembly 9500.

In certain examples, a detachable member 9596 may be a sleeve type member which surrounds the sharp 9586 of a filling implement 9584. Such a detachable member 9596 may fit within and plug the fill port 9524 of the cassette assembly 9500 when a filling implement 9584 is mated with the cassette assembly 9500 and removed. A small orifice may still be present in the fill port 9524 when the detachable member 9596 is retained therein. This may make access through the fill port 9524 difficult and may require very precise aim by the user. Depending on the embodiment, the fill port 9524 may include a barb 9598. This barb 9598 may couple into a recess 9600 included on the detachable member 9596 when the filling implement 9584 is mated with a cassette assembly 9500. The recess 9600 may be present around the entire circumference of the detachable member 9596 so as to allow the filling implement 9584 to be displaced into the fill port 9524 in any rotary orientation. In other embodiments, the recess 9600 may only be present on a portion of the radial face of the detachable member 9596 so as to require a specific rotary orientation or range of orientations in order to access the reservoir 9536. With the barb 9598 coupled into the recess 9600, the detachable member 9596 may be held within the fill port 9524. A sleeve type detachable member 9596 may be capable of only advancing a predefined distance into a fill port 9524 before bottoming out. As a result, a detachable member 9596 may also act as an insertion depth limiter.

In some embodiments, the bore 9602 of the detachable member 9596 may decrease in cross-sectional area, close, or be blocked off when the filling implement 9584 and detachable member 9596 are separated. For example, the bore 9602 may include a complaint material which is compressed when a sharp 9586 is present therein. The compliant material may restore to its resting state when the filling implement 9584 is disassociated with the detachable member 9596. This may close off the bore 9602 and inhibit further access to the reservoir 9536. Alternatively, the detachable member 9596 may include a cantilevered arm which is biased toward the bore 9602. During manufacture, the cantilevered arm may be displaced so as to allow for the sharp 9586 to be extended through the bore 9602. When the detachable member 9596 and filling implement are separated, the cantilevered arm may be urged into the bore 9602 so as to block off the bore 9602 from further access. In some embodiments, the bore 9602 may include a projection which may press against such an arm to prevent the arm from moving out of an obstructing position when the detachable member 9596 is retained in the bore 9602.

In certain examples, a detachable member 9596 such as the sleeve like embodiment described above may include geometric features similar to those described in relation to FIG. 47. Thus, the detachable member 9596 may help ensure use of the filling implement 9584 on which it is included only with intended cassette assemblies 9500. A detachable member 9596 may also include a geometric feature which may catch on a restrictor member present in inappropriate cassette assemblies 9500. When the geometric feature is caught by the restrictor, the sharp 9586 may be prevented from further displacement along the fill port 9524. This may prevent a user from gaining access to a cassette assembly 9500 if the inappropriate filling implement 9584 is employed.

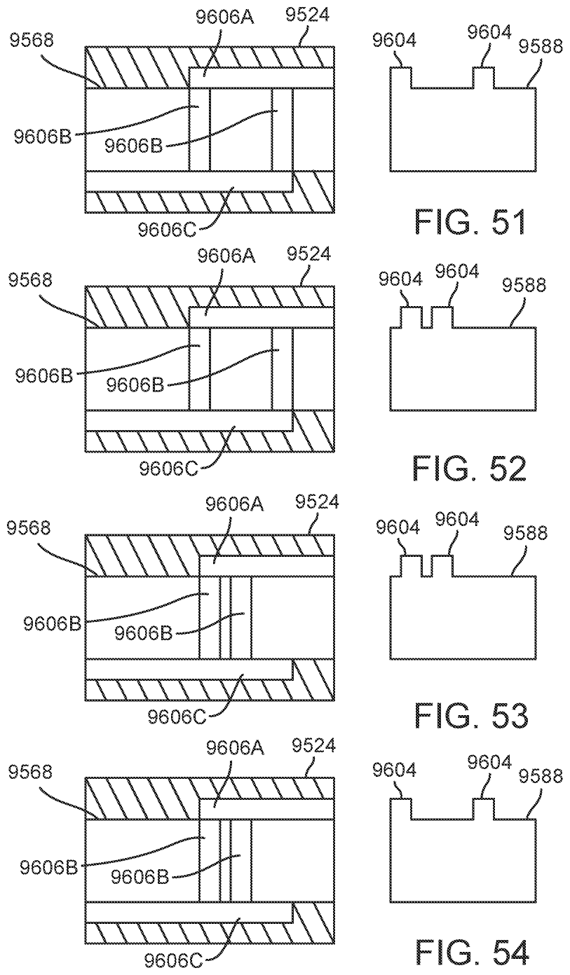

Referring now to FIGS. 51-54, in some embodiments, the interfacing portion 9588 of a filling implement 9584 may be twisted as it is introduced into a mating arrangement with a fill port 9524 or other portion of a cassette assembly 9500. The filling implement 9584 may be incapable of fully coupling for loading of the reservoir 9536 in the cassette assembly 9500 unless such twisting occurs. Where the fill port 9524 cooperates with the interfacing portion 9588 of a filling implement 9584, the fill port 9524 may inhibit twisting during coupling in the event that an improper filling implement 9584 is presented.

In certain embodiments, a filling implement 9584 and fill port 9524 may include cooperative track features which may prescribe a particular displacement path during coupling. For example, the filling implement 9584 may include protuberances or slots on its interfacing portion 9588 which cooperate with channels recessed into the wall of a fill port 9524 bore 9568 or rails extending radially inward from the wall of the fill port 9524 bore 9586. Where multiple types of cassette assemblies 9500 are used (e.g. a U100 type and a U200 type), the track features present in each cassette assembly 9500 type may differ. Filling implements 9584 dedicated for use with each cassette assembly 9500 type may be used and may be incapable of being displaced along the displacement path of the inappropriate cassette assembly 9500.

As shown in FIG. 51, a fill port 9524 and interfacing portion 9588 are depicted. The fill port 9524 and interfacing portion 9588 shown in FIG. 51 are intended for use with one another. The filling implement 9584 interfacing portion 9588 includes a number of protuberances 9604. The bore 9568 of the fill port 9524 may include a number of channels 9606A-C. The spacing, size, number, location, etc. of the channels 9606A-C may be selected to cooperate with a particular interfacing portion 9588 design while inhibiting other interfacing portions 9588 from operatively coupling with the cassette assembly 9500. As shown, the protuberances 9604 of the interfacing portion 9588 shown in FIG. 51 may initially enter channel 9606A when the interfacing portion 9588 is first introduced to the fill port 9606A. The protuberances 9604 may displace along the channel 9606A from their initial entry point to a stop abutting position where the one of the protuberances 9604 may collide with a wall forming the end of the first channel 9606A. In the stop abutting position, further translational displacement of the interfacing portion 9588 may be prevented, but the protuberances 9604 may, be in alignment with twist channels 9606B. The interfacing portion 9588 may be twisted from the stop abutting position to a final channel entry position where the protuberances 9604 enter the final channel 9606C. In the example embodiment, the rotational displacement needed to enter into the final channel 9606C is about 180°. Amounts of rotational displacement may differ in alternative embodiments. Once the protuberances 9604 have reached the final channel entry position, the interfacing portion 9588 may continue to be translationally displaced into a loading position where a sharp 9866 (not shown in FIG. 51) is in communication with the reservoir 9536.

With reference to FIG. 52, the fill port 9524 of FIG. 51 is shown with an inappropriate interfacing portion 9588 of a filling implement 9584. The protuberances 9604 of the interfacing portion 9588 may not be able to align with or pass through at least one of the channels 9606A-C defined in the bore 9568. In the example, the twist channels 9606B would be impossible to align with the protuberances 9604 of the interfacing portion 9588 in FIG. 52. As a result, a sharp 9586 (not shown) included on the interfacing portion 9588 would be prevented from accessing the interior volume of the reservoir 9536.

Referring now to FIG. 53, the interfacing portion 9588 of FIG. 52 is shown with its cooperating fill port 9524. As shown in FIG. 53, when the interfacing portion 9588 is introduced and the protuberances 9604 reach the stop abutting position in the first channel 9606A, the protuberances 9604 may align with the twist channels 9606B included in the bore 9568. The protuberances of the interfacing portion 9588 may be rotated into the final channel entry position and the interfacing portion 9588 may be further introduced into the fill port 9524 such that it reaches the loading position.

Referring now to FIG. 54, the interfacing portion 9588 of FIG. 51 is depicted with the fill port 9524 of FIG. 53. As in FIG. 52, the components depicted are not intended for use with one another. At least one of the protuberances 9604 of the interfacing portion 9588 may not be capable of aligning with the twist channels 9606B. Consequently, a user would not be able to gain access to the interior volume of the reservoir 9536 using the interfacing portion 9588.

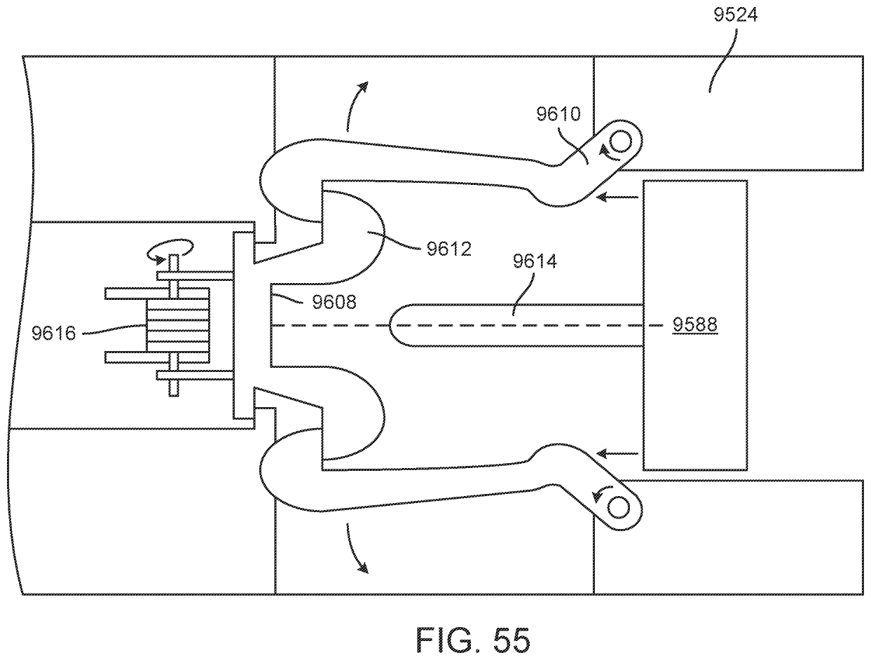

Referring now to FIG. 55, in some embodiments, a door 9608 may be included in the fill port 9524. The door 9608 may be held in place by one or more catches 9610. The door 9608 may include a latch projection 9612 for each of the catches 9610. When the latch projections are engaged with the catches 9610, the door 9608 may be held in place and block access to the interior volume of the reservoir 9536. When a filling implement 9584 is introduced into the fill port 9524 the interfacing portion 9588 of the filling implement 9584 may disengage the catches 9610 from the latch projections 9612 on the door 9608. In the example shown in FIG. 55, the filling implement 9584 includes a blunt cannula 9614 in place of a sharp 9586. When the catches 9610 are disengaged, the blunt cannula 9614 may press against the door 9608 causing the door 9608 to hinge out of a blocking orientation and permit access to the interior volume of the reservoir 9536 for filling. Once the filling implement 9584 is removed, the door 9608 may be urged back to its closed configuration via a bias member 9616.

Where multiple types of cassette assemblies 9500 are used, the fill port 9524 in each may differ. This may allow for each type of cassette assembly 9500 to only be accessible through a filling implement 9584 intended for that cassette assembly 9500. The diameters (or another dimension) of the interfacing portion 9588 of each filling implement 9584 may be selected so that an interfacing portion 9588 may only actuate a door 9608 in a cassette assembly 9500 for which it is intended. For example, an inappropriate filling implement 9584 for the fill port 9524 depicted in FIG. 55 may have a diameter which is smaller than the shortest distance between the two catches 9610. As a result it would be unable to actuate the catches 9610 and unlatch the door 9608. The diameter of the fill port 9524 for another cassette assembly 9500 may be too small to accept the interfacing portion 9588 shown in FIG. 55. As a result, the interfacing portion 9588 shown in FIG. 55 would be incapable of attaching the door 9608 included therein.

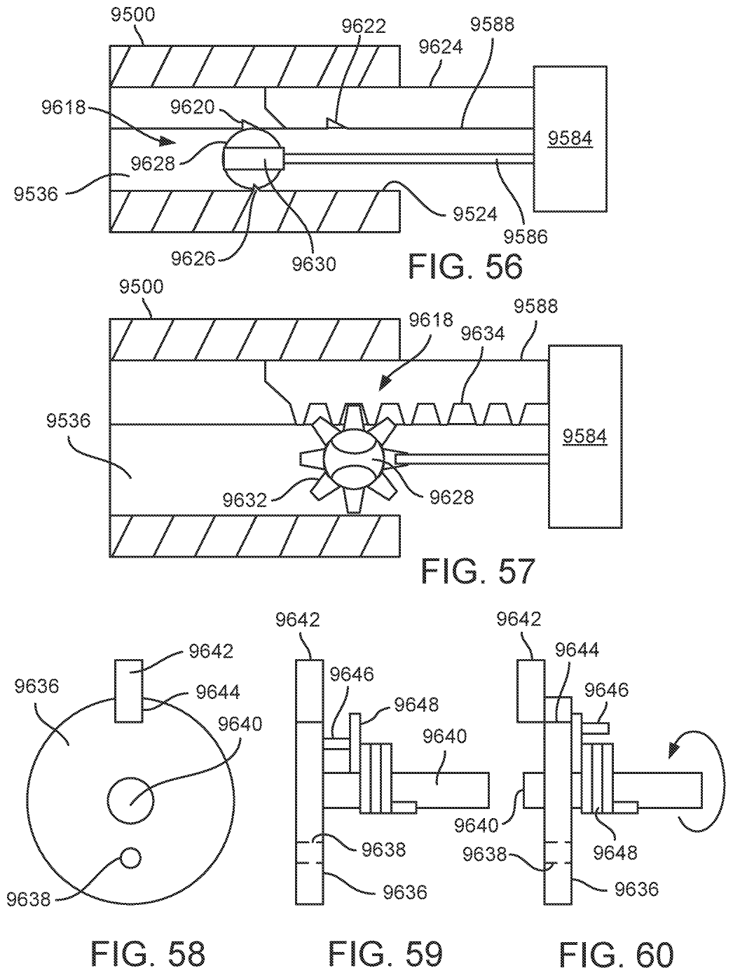

Referring now to FIG. 56, in another embodiment, the fill port 9524 may include a stopcock assembly 9618. The stopcock assembly 9618 may be provided so as to prevent reuse of a cassette assembly 9500. As shown, the stopcock assembly 9618 may include a barb 9620 which may engage with a notch 9622 included in an arm 9624 on the interfacing portion 9588 when the filling implement 9584 is introduced to the fill port 9524. A pawl 9626 may be included to prevent rotation of the valve rotor 9628 of the stopcock assembly 9618 as the filling implement 9584 is inserted into the fill port 9524. The pawl 9626 may engage with a recess in the valve rotor 9628 to inhibit this rotation. After loading of the reservoir 9536 has completed, the user may remove the filling implement 9584. As the filling implement 9584 is withdrawn the notch 9522 may exert a force against the barb 9620 which causes rotation of the valve rotor 9628. As the valve rotor rotates 9628, the flow path 9630 through the valve rotor 9628 may be displaced to a position where the flow path to the interior volume of the reservoir 9536 has been broken. The pawl 9626 may engage another recess in the valve rotor 9528 preventing the valve rotor 9628 from reestablishing a flow path to the interior volume of the reservoir 9536.

Referring now to FIG. 57, in another embodiment, a stopcock assembly 9618 may be included in a cassette assembly 9500 to restrict access to the interior volume of the reservoir 9536. In such embodiments, the valve rotor 9628 may include a pinion portion 9632. The interfacing portion 9588 of the filling implement 9584 may include a rack member 9634 which may engage with the pinion portion 9632 of the stopcock assembly 9618. During introduction of the interfacing portion 9588 of the filling implement 9584, the valve rotor 9628 may be rotated from a blocking orientation into an access permitting orientation. After the reservoir 9536 has been loaded, removal of the filling implement 9584 may rotate the valve rotor 9628 back to the blocking orientation. In the event that an improper filling implement 9584 was utilized by the user, the valve rotor 9628 would not be rotated and the flow path to the interior volume of the reservoir 9536 may remain blocked. As a result, the rack 9634 and pinion 9632 arrangement may restrict access to the reservoir 9536 when an inappropriate filling implement 9584 is employed.

Referring now to FIGS. 58-60, in certain examples, the fill port 9524 may include a disc 9636 which is rotatable about a pivot 9640 and disposed at least partially within the bore 9568 of the fill port 9524. The disc 9636 may prevent reuse of a cassette assembly 9500. The disc 9636 may include an aperture 9638 which may align with a channel leading to the interior volume of the reservoir 9536. In some embodiments, the disc 9636 may be rotationally held in place by a catch 9642 which may engage with a notch 9644 included in the disc 9636. The disc 9636 may also include a projection 9646. In some embodiments, a portion of a bias member 9648 may exert a bias force against the projection 9646 when the aperture 9638 is aligned with the channel. The catch 9642 may prevent the disc 9636 from rotating under the force of the bias member 9648. When the interfacing portion 9588 of a filling implement 9584 is introduced into the fill port 9524, the interfacing portion 9588 may push the disc 9636 and the disc 9636 may be translationally displaced into the bore 9568 of the fill port 9524. This may free the notch 9644 from the catch 9642. A blunt cannula 9614 or sharp 9586 may be present in the aperture 9638 and may hold the disc 9636 in its rotational orientation against the force of the bias member 9648 while the reservoir 9536 is being loaded with fluid. Once the filling implement 9584 is removed, the bias force exerted against the projection 9646 by the bias member 9648 may rotate the disc 9636 to an orientation in which the aperture 9638 is out of alignment with the channel to the reservoir 9536. Further attempts to fill the reservoir 9536 may be blocked by the wall of the disc 9636 and a user may be prevented from reusing the cassette assembly 9500.

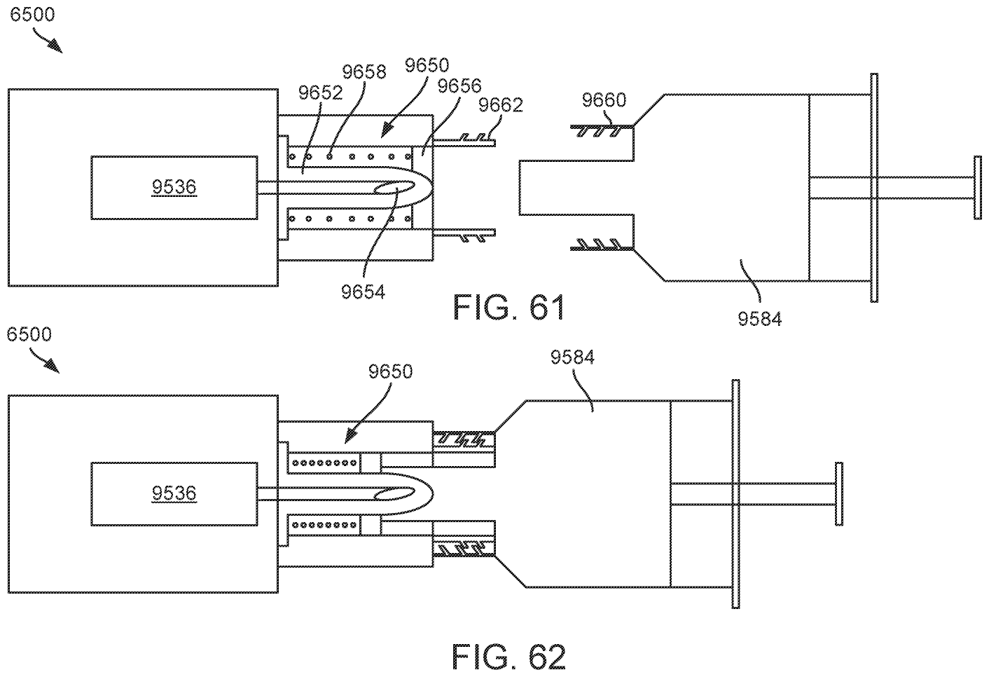

In some examples, and referring now to FIGS. 61-62, the fill port 9524 may include a needleless connector assembly 9650. The needleless connector assembly 9650 may eliminate use of a needle (and any risk of inadvertent needle sticks) when filling a cassette assembly 9500. Such an arrangement may also inhibit establishing a flow path through the fill port 9524 to the interior volume of the reservoir 9536 in the event that a user attempts to access the reservoir 9536 with a generic syringe or needle. Thus a needleless connector assembly 9650 may also act as needle use prevention arrangement. Any variety of needleless connector assembly 9650 may be used. For example, positive, negative, or neutral displacement needleless connector assemblies 9650 may be used. Split septum type needleless connector assemblies may be used. Needless connector assemblies 9650 having internal valves other than a split septum may also be used. Internal valves may for example be fluid pressure actuated or mechanical valves with moving components. The needleless connector assembly 9650 may seal the fill port 9524 when not in use and may be opened when the filling implement 9584 is introduced or coupled to the fill port 9524. In some examples, the needless connector assembly 9650 may be luer activated and open when a luer connection between the filling implement 9584 and fill port 9524 is established.

Where different types of cassette assemblies 9500 may be used, the needleless connector assembly 9650 included on each type may be configured to work only with an appropriate filling implement 9584. An inappropriate filling implement 9584 may not open or be able to engage with the wrong needleless connector assembly 9650. Using insulin as an example, there may be a needleless connector assembly 9650 specific to U100 insulin and a different needleless connector assembly 9650 specific to U200 insulin. A U100 filling implement 9584 or cassette assembly 9500 may not be operable with the respective U200 cassette assembly 9500 or filling implement 9584. This may be accomplished via size differences, geometry differences, or otherwise ensured by the design of the needleless connector assembly 9650.

In the example embodiment, (See FIGS. 61-62) the needless connector assembly 9650 includes an internal blunt cannula 9652 which includes an opening 9654. A portion of the internal blunt cannula 9652 may be surrounded by a displaceable closure member 9656. A bias member 9658 may be included to bias the closure member 9656 toward a closed position in which a seal is formed around the opening 9654 in the blunt cannula 9652. Upon connection to a proper filling implement 9584, the closure member 9656 may be pressed away from the closed state into an access permitting state. As shown, the cassette assembly 9500 and filling implement 9584 include cooperating coupler fittings 9660, 9662. Luer fittings or barbed fittings may for example be used. When the filling implement 9584 coupler fitting 9660 is threaded into engagement with the cassette assembly 9500 coupler fitting 9662, the outlet port 9664 of the filling implement 9584 may cause the displaceable closure member 9656 to actuate to the access permitting state. A portion of the blunt cannula 9652 may sealingly engage the outlet port 9664 of the filling implement 9584 and fluid may be delivered into the interior volume of the reservoir 9536. Upon disengagement of the filling implement 9584 with the fill port 9524 the closure member 9656 may be urged back to the closed state by the bias member 9658.

Referring now to FIG. 63, in some examples a “smart” filling implement 9584 and cassette assembly 9500 may be used. One of the filling implement 9584 and the cassette assembly 9500 may include a wireless ID tag 9666 (e.g. RFID, NFC, UHF, HF, etc.) and the other may include an interrogator 9668. Typically, and as shown, the tag 9666 may be included in the cassette assembly 9500. The tag 9666 use may be passive tag which includes no on board power source. In other embodiments, active tags 9666 are also possible. The tag 9666 may be encoded with information identifying the type of cassette assembly 9500 (e.g. U100 or U200 using insulin as an example), size of the reservoir 9536, refill interval, etc.

As mentioned above, the filling implement 9584 may include an interrogator 9668. The interrogator 9668 may check the tag 9666 included as part of the cassette assembly 9500. A controller 9670 included in the filling implement 9584 may verify that the tag 9666 indicates that the cassette assembly 9500 is appropriate for use with the filling implement 9584. In the event that the tag 9666 is for a cassette assembly 9500 not intended for use with that filling implement 9584, the controller 9670 may not command fluid to be dispensed from the filling implement 9584. Alternatively, the filling implement 9584 may be communicatively linked with another component of the delivery system 10 (e.g. the reusable housing assembly 106) and may receive data related to the therapy about to be conducted therefrom. In the event that the therapy program defines a cassette assembly 9500 type that does not match the tag 9666, the controller 9670 may prevent any fluid from being output by the filling implement 9584.

In embodiments where the cassette assembly 9500 is not intended for reuse, the controller 9670 may check to determine whether the tag 9666 belongs to a cassette assembly 9500 which has been previously used. For example, the controller 9670 may query a memory (not shown) of the filling implement 9584 to determine whether a unique identifier encoded in the tag 9666 matches any identifiers previous scanned by the interrogator 9668. In the event that reuse of a cassette assembly 9500 is attempted, the filling implement 9584 may not permit dispensation of fluid. In other embodiments, a remote database (e.g. cloud server) may be communicatively linked to the filling implement 9584. The filling implement 9584 may perform a reuse prevention check by determining if the unique identifier on the cassette assembly 9500 is listed in the database as having been previously used. In embodiments where reuse is permitted, the filling implement 9584 may check an error history associated with the unique identifier of the cassette assembly 9500. In the event that a previous usage of the cassette assembly 9500 was associated with certain errors or patterns of errors (e.g. leak detected, occlusion, repeated occlusions, or such types of errors followed by a discontinuation of therapy), the filling implement 9584 may prevent loading of fluid into the reservoir 9536 of the cassette assembly 9500. In the event that the controller 9670 determines an acceptable cassette assembly 9500 has been presented, the controller 9670 may command or allow fluid to be filled into the reservoir 9536.

In the example embodiment, the filling implement 9584 is depicted with a pump 9672. Any type of pump 9672 may be used. The filling implement 9584 may command dispensing of a specified volume of fluid into the reservoir 9536 via the pump 9672. The specified volume of fluid may be encoded in the tag 9666 or alternatively may be a preset volume appropriate for the cassette assembly 9500 being used. The volume dispensed into the reservoir 9536 may be communicated by the filling implement 9584 may be communicated to other components of the delivery system 10. For example, the filling implement 9584 may communicate the volume filled into the reservoir 9536 to a reusable housing assembly 106. This volume may then be used to determine volume remaining in the reservoir 9536 as a therapy is executed by the drug delivery system 10.

In alternative embodiments, the tag 9666 may be replaced by an indicium (printed indicium, barcode, QR code, data matrix, bokode, digimarc, etc.). The filling implement 9584 may include a reader (e.g. camera, barcode scanner, etc.) which may read the indicium. The controller 9670 may use data from the indicia as described above to help ensure that fluid in the filling implement 9584 is only dispensed into appropriate cassette assemblies 9500. Additionally, in some embodiments, the interrogator 9668 or reader may not be included in the filling implement 9584. Instead, the interrogator 9668 or reader may be included in another component of the delivery system 10 such as the reusable housing assembly 106 or communicatively linked device 124 such as a smart phone.

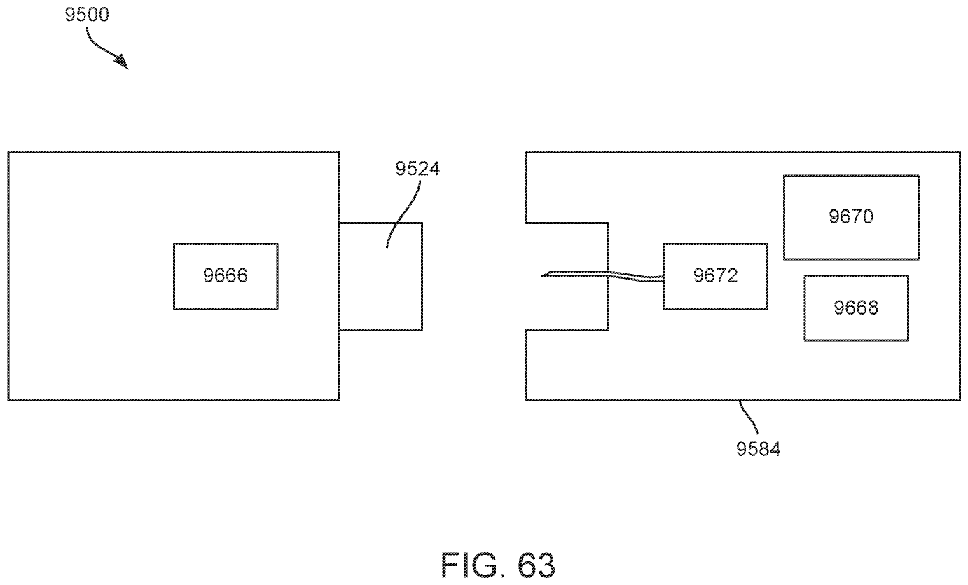

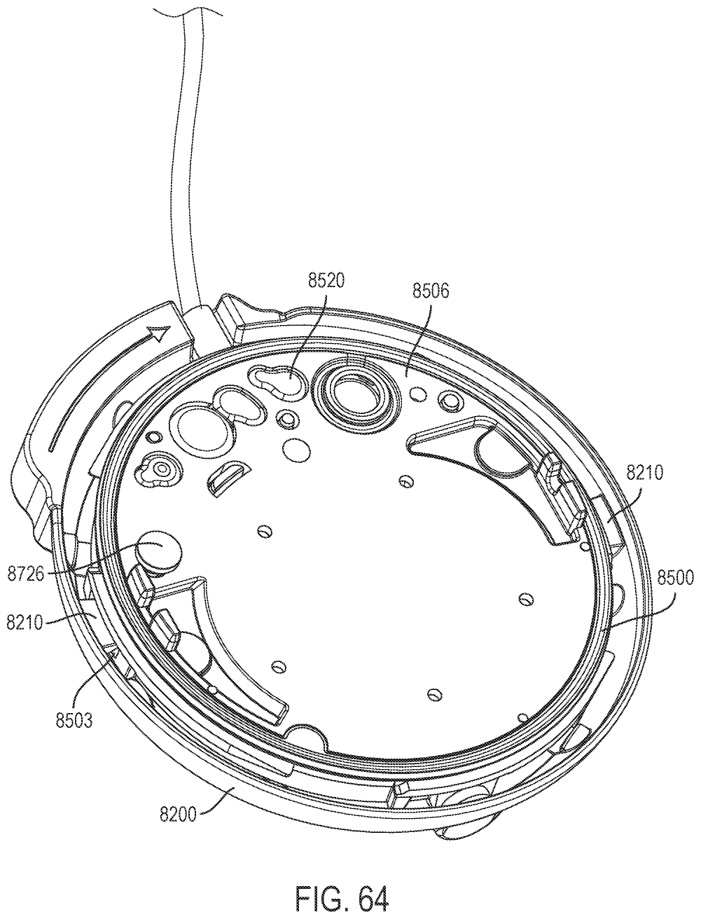

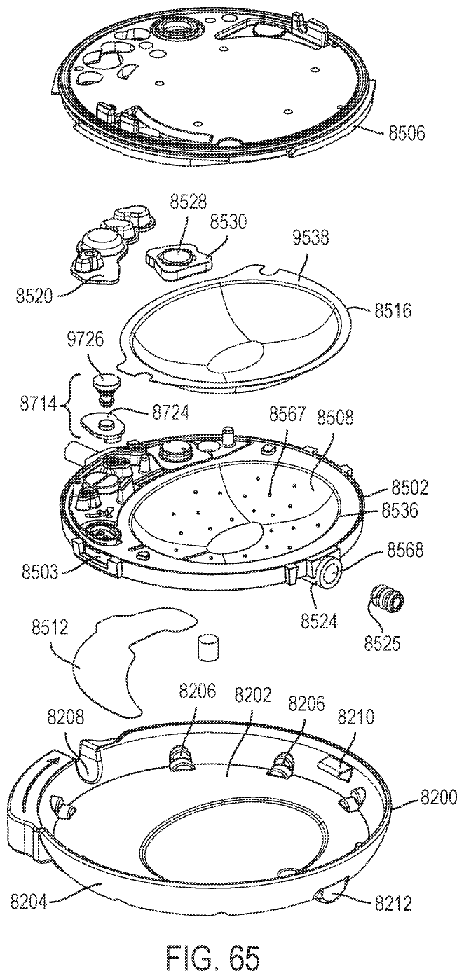

Referring now to FIGS. 64, 65 various views of an exemplary disposable cassette assembly 8500 is depicted. The cassette assembly is releasably engageable with the reusable housing 106. The cassette assembly 8500 includes a cassette base portion 8502. The base cassette base portion 8502 may include a reservoir recess 8508 which may be formed integrally therein. The cassette base portion 8502 of a prefilled cassette assembly 8500 may be formed from a long term drug compatible material such as a cyclic Olefin Polymer (COP), an example such as Zeonor® 1020R. Where the cassette assembly 8500 is user filled, the cassette base portion 8502 may be made of a Cyclic Olefin Copolymer (COC) such as Topas®, or of a polyester such as Tritan®. The reservoir recess 8508 may be covered by a piece of reservoir film 8516 which is coupled to the base cassette base portion 8502. Together, the reservoir recess 8508 and reservoir film 8516 may define a reservoir 8536 for holding fluid (such as various drugs) in its interior volume. In certain embodiments, the fluid may be a drug for an endocrine disorder. For example, the fluid may be a diabetes management drug such as insulin. Short or rapid acting insulin (e.g. Aspart, Lispro, Glulisine, Velosulin, regular human insulin such as Novolin-R or Humulin R) may for example be used, though longer acting insulins (e.g. detemir, glargine, degludec, Toujeo) may be also be used. Cardiovascular drugs may also be used. For example, vasodilators or anti-hypertensive agents such as treprostinil may be used. Fluids may also include analgesics, chemotherapy drugs, enzymes, pegylated proteins, small molecules, natural products, peptide, proteins, nucleic acids, carbohydrates, nanoparticulate suspensions, and associated pharmaceutically acceptable carrier molecules.

The reservoir film 8516 may be affixed to the cassette base portion 8502 via adhesive, ultrasonic welding, heat sealing, etc. to generate a fluid tight seal between the cassette base portion 8502 and the reservoir film 8516. Alternatively, the reservoir film 8516 and cassette base portion 8502 may be compressively pinched together by the cassette top portion 8506 of the disposable cassette assembly 8500 when the cassette top portion 8506 is coupled into the cassette assembly 8500. In some examples, and as described further later in the specification, the reservoir film 8516 may be affixed to the cassette base portion 8502 and the cassette top portion 8506 and the cassette top portion 8506 may be coupled in place in the cassette assembly 8500 by welding at least a portion of cassette top portion 8506 onto the reservoir film 8516. The cassette top portion 8506 may be made of the same material as the cassette base portion 8502 for improved welding together. The reservoir film 8516 may be constructed of a number of layers of materials which may be selected to add various desirable traits to the reservoir film 8516. Where applicable, tie layers may be used as well. In some embodiments, a drug compatible layer may form the interior volume facing surface of the reservoir film 8516, for example a long term drug compatible material such as a cyclic Olefin Polymer (COP), an example such as Zeonor® 1020R. A barrier layer which is impermeable to gas or specific gases may be included outward of the compatibility layer. The intermediate or tie layer may be an anethylene vinyl alcohol (EVOH). The outer layer may be polychlorotrifluoroethylene (PCTFE) such as Aclar®. Where layered film is used, the reservoir film 9516 may be a coextruded product. In alternative embodiments, the reservoir film 8516 may be constructed of nitrile, silicone, or chlorobuytl rubber.

In the example embodiment, the reservoir film 8516 may include a preform region 8536 (best shown in FIG. 65). The preformed region 8534 may be vacuum or thermoformed. In the example embodiment, the preformed region 8534 is shown as a depression. The depression may be bowl like and may mimic the shape of the reservoir recess 8508 included in the cassette base portion 8502 of the disposable cassette assembly 8500. When the reservoir film 8516 is coupled to the disposable cassette assembly 8500, the preformed region 8534 may seat within the reservoir recess 8508 such that the reservoir film 8516 abuts the bottom surface of the reservoir recess 8508 and sits against the recess wall 8510. This may ensure that minimal air volume is present in the reservoir 8536 (see, e.g. FIG. 19) prior to filling of the reservoir 8536. In the example embodiment, the reservoir recess 8508 includes a series of dimples 8567 molded into the reservoir bottom. The dimples 8567 extend into the reservoir cavity and may keep the reservoir film 8515 from fully adhering to the bottom and sides of the reservoir recess 8508, thereby creating channels to allow fluid to flow therebetween and allow for more complete emptying of the reservoir.

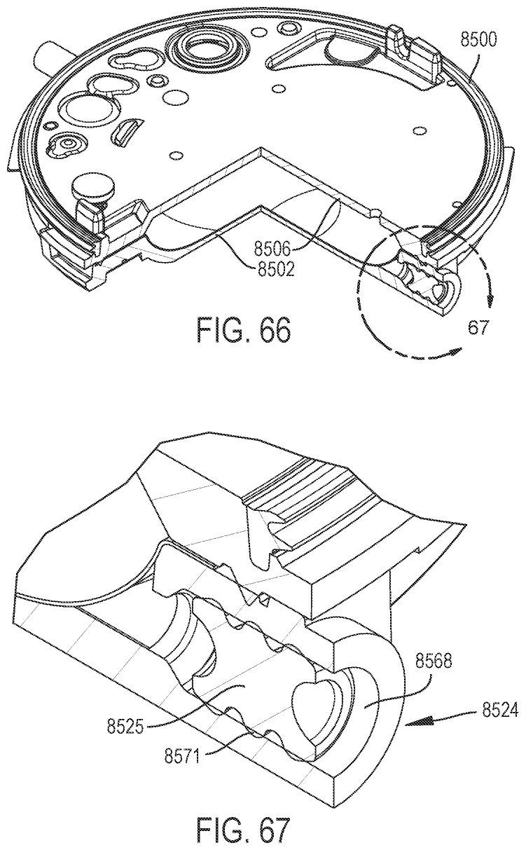

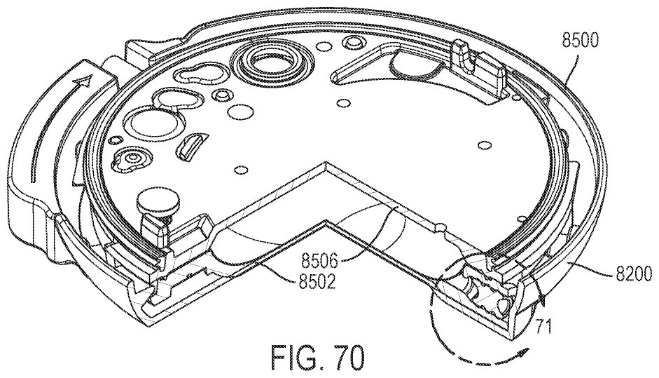

A fill port 8524 may also be included in the cassette base portion 8502. In the example embodiment, the fill port 8542 extends through a sidewall of the cassette base portion 8502. The fill port 8524 may extend directly to the reservoir 8536 (see, e.g. FIG. 70) and may be sealed with a plug 8525 (See FIGS. 66-67). The plug 8525 can be made of a material compatible with long term drug storage. The area of the plug 8525 exposed to the drug is small, therefore the plug can be formed from halogenated butyl rubber, bromobutyl, or chlorobutyl.

The reservoir film 8516 may include a peripheral rim region 8538. The peripheral rim region 8538 may be coupled to the cassette base portion 8502 at an attachment surface 8540 of the cassette base portion 8502 which may surround the reservoir recess 8508. In some examples, an outcrop 8542 may be included in the peripheral rim region 8538. Where the cassette top portion 8506 is welded onto the reservoir film 8516.

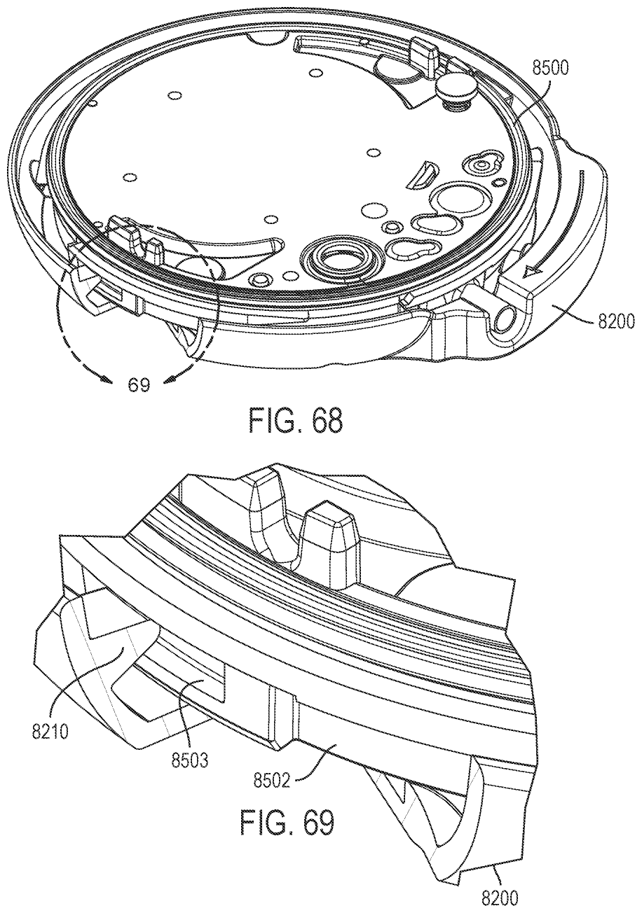

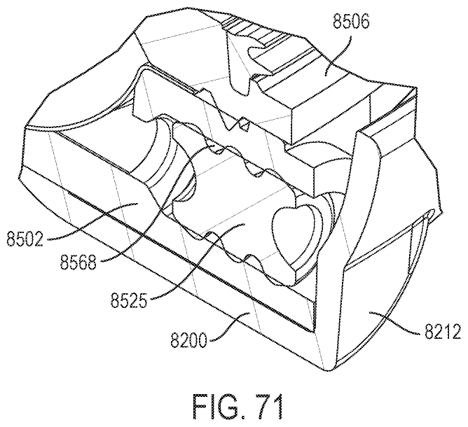

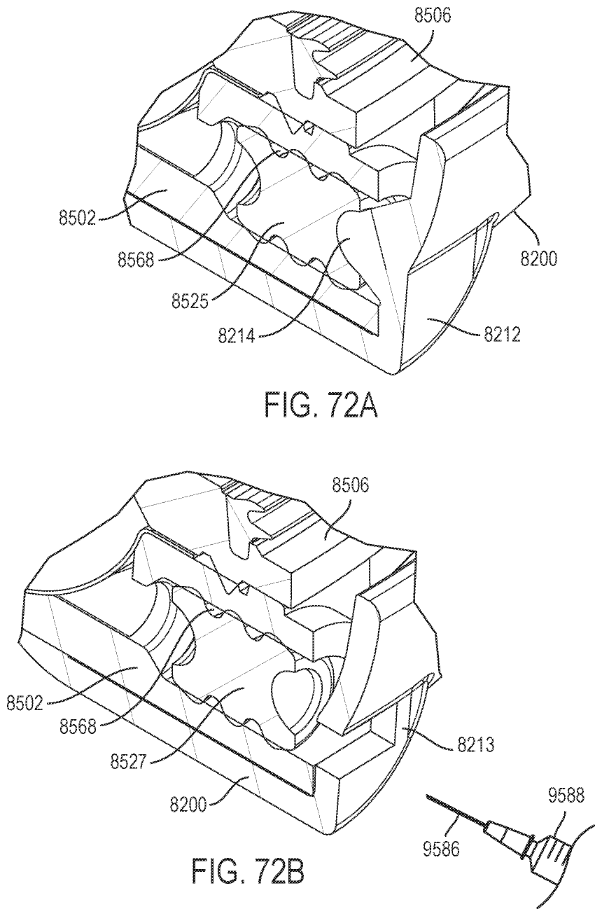

The cassette assembly 8500 has a cassette shell 8200 that attaches to the cassette base portion 8502. The cassette shell 8200 forms a base for contact with the user. The cassette shell 8200 may be made of an extruded material such as plastic compatible with contact with the user skin and body oils, lotions and other materials. The cassette shell 8200 can be made of an Acrylonitrile Butadiene Styrene (ABS) such as Lustran® 348. The use of a cassette shell 8200 allows the cassette base portion 8502 and cassette top portion 8506 to be made of materials that are long term drug compatible while the cassette shell 8200 can be made of materials resistant to sweat, body oils, lotions and other materials that may be present on the human body. The cassette shell 8200 has a bottom 8202 and a circumferential shell wall 8204. The cassette shell 8200 may define a series of standoffs 8206 around the inside of the cassette shell for positioning the cassette base portion 8502 and cassette top portion 8506 away from the shell bottom 8202 and shell wall 8204. The cassette shell wall 8204 may define a cutout 8208 for extension of a lure or tubing 184 from the cassette base portion 8502. From the inside of the shell wall 8204 may extend tabs 8210 for snap fit engagement with slots 8503 defined in the cassette base portion 8502. (See FIGS. 68, 69) The cassette shell 8200 is sufficiently resiliently deformable to allow the taps 8210 to fitted into the slots 8503 and thereby secure the cassette shell 8200 to the cassette base portion 8502. Further, the cassette shell 8200 may define a reservoir plug stop 8212 to aid in securing the reservoir plug 8525 in the reservoir opening 8524. The reservoir plug stop 8212 is positioned close to or in contact with the reservoir plug opening 8524 when the cassette shell 8200 is secured to the cassette base portion 8502. (See FIG. 71) In a further embodiment, the reservoir plug stop 8212 may define a plug cover extension 8214 that extends into the reservoir fill bore 8568 to contact the reservoir plug 8525. (See FIG. 72A) The reservoir plug stop 8212 provides an additional measure of securing the reservoir plug in place for transport of a pre-filled cassette assembly 8500 from location of filling to the end user. In another embodiment (See FIG. 72B) which may be intended for refilling or user filling, the cassette shell 8200 defines a reservoir plug opening 8213 opposite the reservoir plug opening 8524. The reservoir plug opening is filled with a septum 8527 that permits the use of the sharp 9586, of filling implement 9584, to pierce the septum 8527 and fill the reservoir 8536.

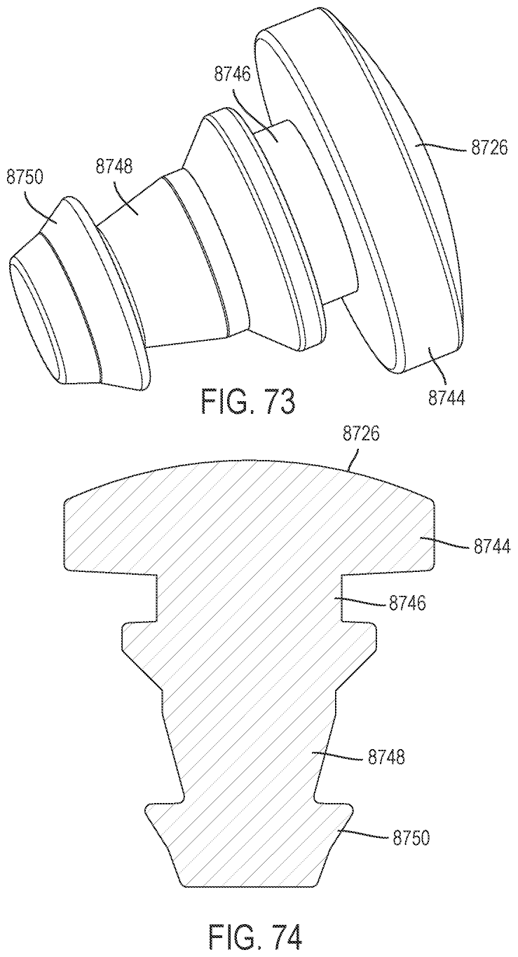

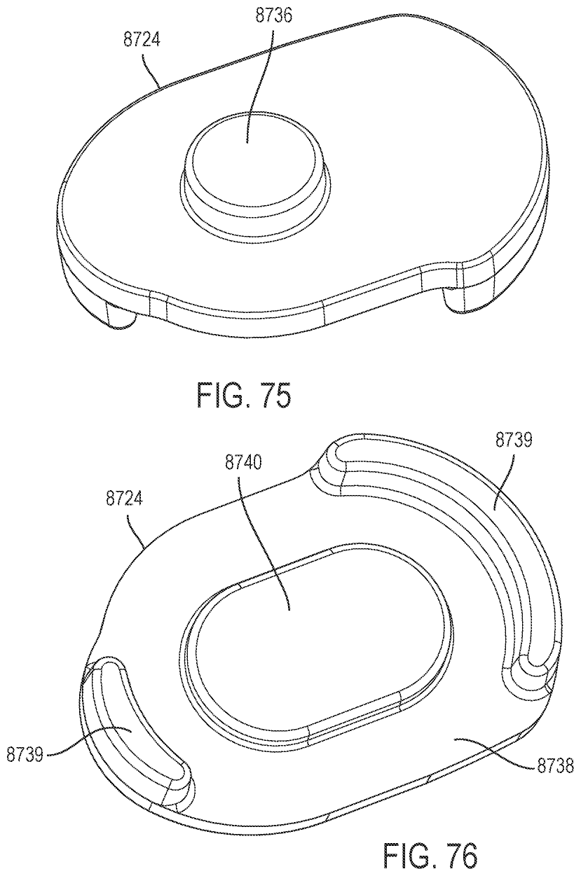

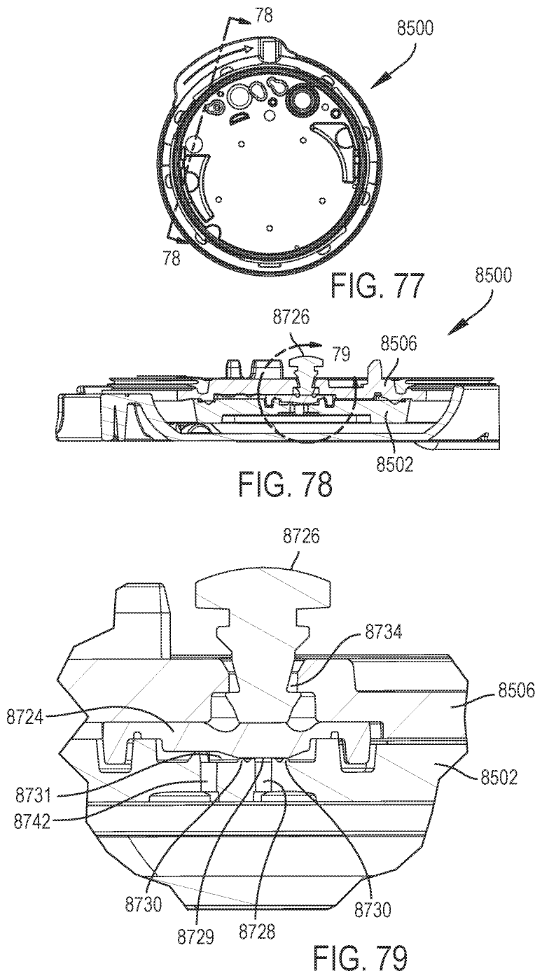

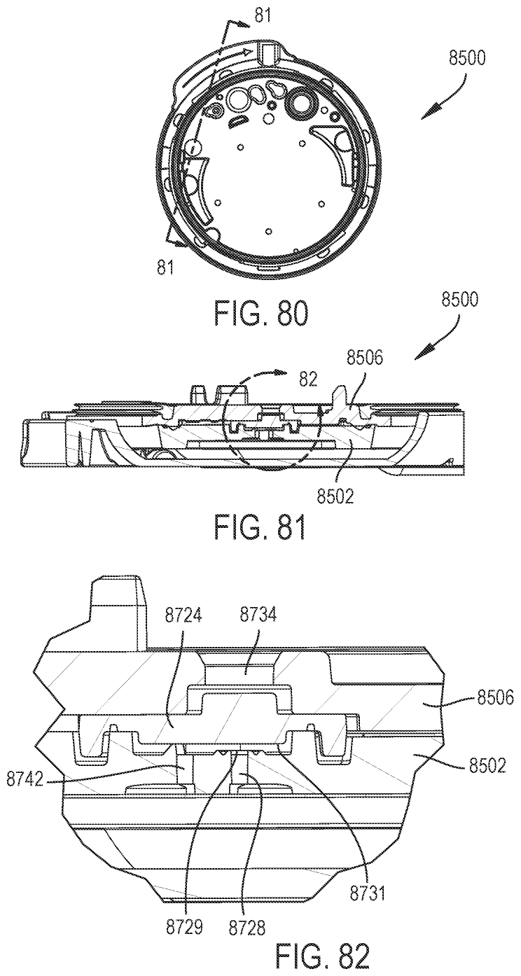

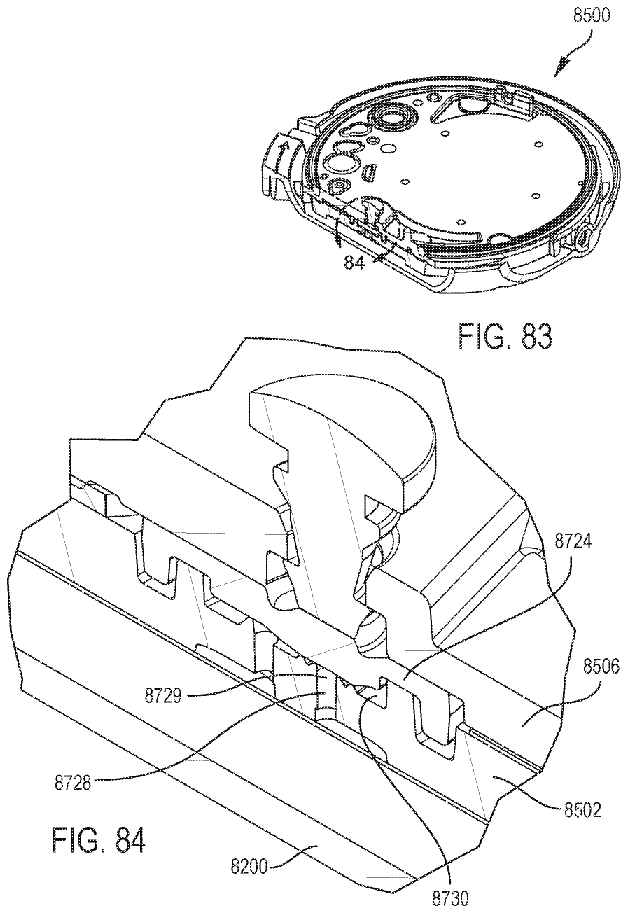

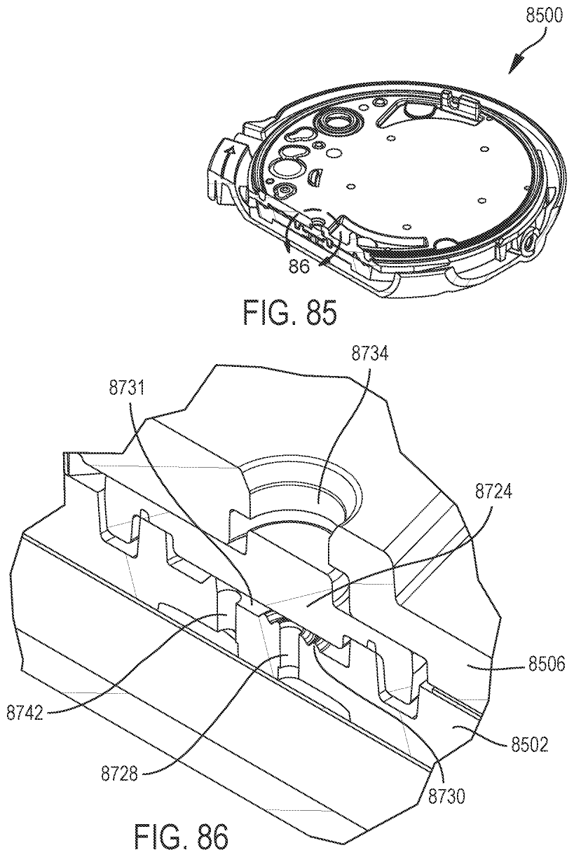

Cassette assembly 8500 further includes a valve membrane 9520 and volume sensor diaphragm assembly 9526 as described above for cassette assembly 9500. With reference to FIGS. 73-86, an occluder assembly 8714 has an occluder diaphragm 8724 and a bung or occluder actuator 8726. The occluder diaphragm 8724 can be made of a material compatible with long term drug storage. The area of the occluder diaphragm 8724 5 exposed to the drug is small, therefore the diaphragm can be formed from halogenated butyl rubber, bromobutyl, or chlorobutyl. The cassette base portion 8502 defines an inlet fluid passage 8728 from the reservoir 118 to an occluder chamber 8731. The cassette base portion 8502 further defines a second outlet fluid passage 8742 from the occluder chamber 8731 to the inlet valve assembly 614. The occluder fluid inlet opening 8729 is surrounded by a pair of annular sealing ridges 8730. A circumferential and closed occluder chamber wall 8732, formed by the cassette base portion 8502, surrounds the occluder chamber 8731 and has a top surface for sealing with the occluder diaphragm 8724. The occluder diaphragm 8724 is captured between the cassette base portion 8502 and the cassette top portion 8506. The cassette top portion defines an annular occluder actuator opening 8734 for receiving the occluder actuator 8726. With particular reference to FIGS. 75 and 76, the occluder diaphragm 8724 has a top surface with an occluder diaphragm extension 8736 for contact with the occluder actuator 8726. When the occluder diaphragm 8724 is captured between the cassette base portion 8502 and cassette top portion 8506, the occluder diaphragm extension 8736 is aligned with the occluder actuator opening 8734. The bottom surface of the occluder diaphragm 8724 has an outer wall sealing surface 8738 for sealing with the top surface of the occluder chamber wall 8732. The thickness of the occluder diaphragm 8724 is sufficient so when the occluder diaphragm 8724 is captured between the cassette base portion 8502 and cassette top portion 8506, the wall sealing surface 8738 forms a fluid tight seal for the occluder chamber 8731 with the occluder chamber wall 8732. The bottom of the occluder diaphragm 8724 defines an inner inlet opening sealing surface 8740 for sealing with the sealing ridges 8730. The occluder diaphragm 8724 further may have a pair of locating ridges 8739 for locating the occluder diaphragm over the occluder chamber 8731 during the assembly process of the top cassette portion 8506 with the cassette bottom portion 8502.

The bung or occluder actuator 8726 (See FIGS. 73, 74) is radially formed of a resiliently deformable material and has an occluder actuator head portion 8744, an occluder actuator upper neck portion 8746, an occluder actuator lower neck portion 8748 and a lower occluder actuator catch portion 8750. When the occluder assembly 8714 is in the closed position, occluding the flow of fluid, the occluder actuator 8726 is positioned in the occluder actuator opening 8734. The catch portion 8750 contacts the occluder diaphragm extension 8736 and deforms the occluder diaphragm so the inlet opening sealing surface it brought into contact with the sealing ridges 8730 to form a fluid tight seal and thereby fluidly isolate the reservoir from the downstream fluid passages in the cassette assembly 8500. (See FIGS. 79 and 84.) The use of multiple annular continuous rings for the sealing ridges 8730 provides multiple seals so as to improve sealing during transport of the cassette assembly 8500. The occluder actuator catch portion 8750 has a diameter sufficiently larger than the occluder actuator opening 8734 whereby the occluder actuator 8726 can deform to be inserted through the occluder actuator opening 8734, but also sufficiently large that the force of the deformable occluder diaphragm 8724 wanting to return to its rest state is insufficient to drive the occluder actuator from the occluder actuator opening 8734. The catch portion 8750 may be further shaped with a flat annular surface perpendicular to the axis of the occluder actuator 8726 to improve contact with the rim area of the occluder actuator opening 8734. The occluder actuator lower neck portion is generally of similar diameter or less diameter than the occluder actuator opening 8734.

The occluder actuator upper neck portion 8746 defines a neck area having a diameter less than the diameter of the head portion 8744. A tether 8760 may be secured to the upper neck portion, looping the circumference of the upper neck portion 8746 or otherwise secured. The tether 8760 is a flexible filament. The tether 8760 is used to pull and remove the occluder actuator 8726 from the occluder actuator opening 8734. On removal of the occluder actuator 8726, the occluder diaphragm 8724 returns to the undeformed state, thereby releasing the seal of with the sealing ridges 8730 and placing the occluder assembly 8714 in the open state whereby fluid may from the reservoir into the other fluid passages of the cassette assembly 8500. (See FIGS. 82 and 86). The occluder actuator head portion 8726 may additionally be of sufficient size so as to interfere with engagement of the cassette assembly 8500 with the reusable housing 106 when the occluder actuator 7826 is in the occluder actuator opening 8734. This interference can be used to prevent the end user from inadvertently attempting to use the drug delivery system 10 without first having removed the occluder actuator 7826 and thereby allowing the reservoir to provide fluid to the drug delivery system 10 for infusion to the user.

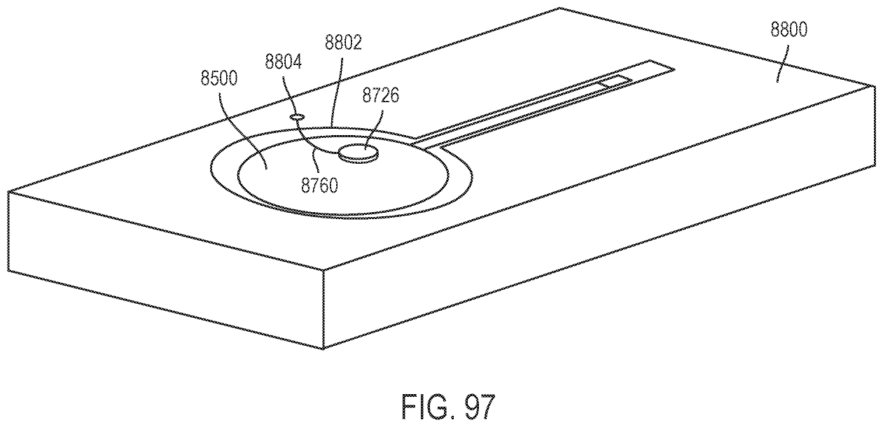

The cassette assembly 8500 maybe prefilled and shipped to the user in a cassette package 8800. (See FIG. 97) The cassette package 8800 defines a cassette package opening 8802 for receiving and securely holding the cassette assembly 8500. A first end of the tether 8760 is secured to the occluder actuator 8726, and the second end of the tether 8760 is secured to a tether anchor 8804 on the cassette package 8800. In use, the end user removes the cassette assembly 8500 from the cassette package 8800. The removal tensions the tether 8760, pulling the occluder actuator 7826 from the occluder actuator opening 8734 and moving the occluder assembly 8714 from the closed state to the open state. The cassette assembly may then be engaged to the reusable housing 106, the auto-prime function initiated, and other necessary steps taken to begin drug delivery to a user.

It should be noted that various access restricting and reuse prevention arrangements described herein are not mutually exclusive of one another. One or more of the concepts shown herein can be used together in a single embodiment. Access may be controlled and reuse may be prevented by any desired combination of the above.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

What is claimed is:

1.-136. (canceled)

137. A cassette for a drug delivery device comprising:
a cassette base portion including a reservoir recess surrounded by an attachment surface, the attachment surface including a reservoir outlet flow path recessed therein; and a reservoir film coupled to the attachment surface and, together with the reservoir recess, defining a flexible reservoir, a portion of the reservoir film having a preformed shape which mimics the contour of the reservoir recess, the preform shape causing the reservoir film to be disposed adjacent the surface of reservoir recess when the reservoir is in an empty state; and
at least one duct recessed into the surface of the reservoir recess, each of the at least one duct defining a flow path which remains in fluid communication with the reservoir outlet flow path when the reservoir is in an empty state.

138. The cassette of claim 137, wherein the reservoir film comprises an outcrop region that is included in the reservoir film and forms a seal over the reservoir outlet flow path when the reservoir film is coupled to the attachment surface.

139. The cassette of claim 137, wherein the reservoir film includes at least two layers.

140. The cassette of claim 137, wherein the reservoir film is heat bonded to the attachment surface.

141. The cassette of claim 137, wherein the cassette base portion includes a side wall having a fill port leading to an interior volume of the reservoir.

142. The cassette of claim 141, wherein a basin is recessed into the reservoir recess directly downstream of an inlet leading to the interior volume of the reservoir from the fill port.

143. The cassette of claim 142, wherein at least one of the at least one duct extends into communication with the basin.

144. The cassette of claim 138, wherein the at least one duct includes a plurality of ducts which that converge together at a confluence, the confluence disposed intermediate the reservoir outlet flow path and the ducts.

145. The cassette of claim 137, wherein the at least one duct includes a first duct and a second duct which furcates off of the first duct.

146. The cassette of claim 137, wherein the reservoir recess includes a wall which extends from a bottom surface of the reservoir recess to the attachment surface and the at least one duct includes a duct which is disposed along a portion of a perimeter of the bottom surface adjacent the wall.

147. The cassette of claim 146, wherein the duct disposed along a portion of the perimeter is disposed along a majority of the perimeter.

148. The cassette of claim 137, wherein the reservoir recess includes a wall which extends from a bottom surface of the reservoir recess to the attachment surface and the at least one duct includes a portion recessed into the bottom surface and a second portion recessed into the wall.

149. The cassette of claim 148, wherein the first portion of the at least one duct has a first width and the second portion of the at least one duct has a variable width.

150. The cassette of claim 148, wherein the width of the second portion of the at least one duct tapers from a first width to a second width smaller than the first width.

151. The cassette of claim 148, wherein the first portion of the at least one duct is the second width.

152. The cassette of claim 138, wherein the at least one duct includes a plurality of ducts which extend from a confluence region at regular angular intervals, the confluence region being disposed intermediate the plurality of ducts and the reservoir outlet flow path.

153. The cassette of claim 137, wherein the reservoir outlet flow path includes an air trap.

154. A cassette for a drug delivery device comprising:
a cassette base portion including a reservoir recess surrounded by an attachment surface, the cassette base portion further including a weld surface along a portion of the periphery of the cassette base portion and a plurality of locating pins;
a reservoir film coupled to the attachment surface and, together with the reservoir recess, defining a flexible reservoir; and
a cassette top portion including a number of locating pin receptacles and a peripheral energy director, the energy director being aligned with the weld surface and a portion of the reservoir film bonded to the attachment surface when the locating pins are disposed within the locating pin receptacles, the energy director being welded to the weld surface and the portion of the reservoir film.

155. The cassette of claim 154, wherein the energy director comprises a triangular cross section.

156. The cassette of claim 154, wherein the energy director is sonically welded to the weld surface and the portion of the reservoir film.

157. The cassette of claim 154, wherein the energy director is ultrasonically welded to the weld surface and the portion of the reservoir film.

158. The cassette of claim 154, wherein the reservoir film includes at least two layers.

159. The cassette of claim 154, wherein the cassette top portion includes a bay including a number of retention tabs.

160. The cassette of claim 159, wherein the cassette further comprises a cover having a number of latch clips, the latch clips configured to cooperatively engage the retention tabs of the bay, the cover being coupled to the cassette top portion when the latch clips cooperatively engage the retention tabs.
